(19) United States
(12) Patent Application Publication
(10) Pub. No.: US 2007/0087352 A9
(43) Pub. Date: Apr. 19, 2007

(54) Inhibitors for androgen antagonist refractory prostate cancer

(76) Inventors: Robert Fletterick, Hillsborough, CA (US); Eva Estebanez-Perpina, San Francisco, CA (US); Eugene Hur, San Francisco, CA (US); Samuel James Pfaff, San Francisco, CA (US); Benjamin Marcus Buehrer, Chapel Hill, NC (US)

Correspondence Address: FISH & RICHARDSON P.C., PO BOX 1022, MINNEAPOLIS, MN, 55440-1022, US

(21) Appl. No.: 10/705,165

(22) Filed: Nov. 6, 2003

(51) Int. Cl. C12Q 1/68; G06F 19/00; G01N 33/48; G01N 33/50

(52) U.S. Cl. 435/6; 702/19

(57) ABSTRACT

The present invention relates to methods and antagonist compounds for modulating androgen receptor activity. The invention includes a method for identifying molecules that bind to a coactivator binding site of a receptor in the androgen receptor family. Also included is a cocrystal of an androgen receptor ligand binding domain complexed with a ligand and a coactivator. The invention further includes a method for inhibiting androgen receptor activity in a mammal, thereby facilitating treatment of diseases such as prostate cancer.

### RELATED APPLICATIONS

This application is related to U.S. patent application Ser. No. 09/281,717, filed Mar. 30, 1999, and to provisional applications, Ser. No. 60/079,956, filed Mar. 30, 1998, and Ser. No. 60/113,146, filed Dec. 16, 1998, all of which are incorporated herein by reference in their entirety.

This application is also related to U.S. patent application Ser. No. 09/609,361, filed Jun. 30, 2000, and Ser. No. 09/830,693, filed Mar. 30, 1999, and U.S. provisional application Ser. No. 60/113,014, filed Dec. 16, 1998, all of which are incorporated herein by reference in their entirety.

### ACKNOWLEDGMENT

This invention was made with Government support under Grant No. CA95324, awarded by the National Institutes of Health. The U.S. Government has certain rights in this invention.

### MATERIALS ON CD-R

This application further comprises tables 1 and 2 presented respectively in the following ASCII format text files herewith on two (2) compact discs one of which is an exact copy of the other, all of which are incorporated herein by reference in their entirety:
Table1_ARLBD_DHT_CDP.txt 342 kbytes created on Nov. 6, 2003

Table2_ARLBD_DHT_CRP.txt 1328 kbytes created on Nov. 6, 2003

LENGTHY TABLES FILED ON CD
The patent application contains a lengthy table section. A copy of the table is available in electronic form from the USPTO web site (http://seqdata.uspto.gov/?pageRequest=docDetail&DocID=US20070087352A9). An electronic copy of the table will also be available from the USPTO upon request and payment of the fee set forth in 37 CFR 1.19(b)(3).

### FIELD OF THE INVENTION

The present invention relates to methods and compounds for modulating nuclear receptor function. In particular, the present invention relates to methods and compounds for treating prostate cancer by inhibiting androgen receptor coactivator binding.

### BACKGROUND

Prostate cancer is the second leading cause of cancer deaths among men in the United States, and it has a complex etiology (see, e.g., Nelson, K. A., and Witte, J. S., “Androgen Receptor CAG Repeats and Prostate Cancer”, Am. J. Epidemiology, 155:883-890, (2002)). Hormone refractory prostate cancer (HRPC) is a prostate cancer that is resistant to forms of hormone therapy.

In general, cells contain receptors, on the surface of proteins, that can elicit a biological response by binding various molecules including other proteins, hormones and drugs. Such responses underpin cellular function, including the uncontrolled replication that is the basis of tumor growth. The androgen receptor (AR) is an intra-cellular receptor that has been implicated in prostate cancer growth. In particular, unregulated AR activity is implicated in metastatic prostate cancers (see, Tenbaum, S., and Baniahmad, A., “Nuclear Hormone Receptors: Structure, Function and Involvement in Disease,” Int. J. Biochem. and Cell Biol., 29:1325-1341, (1997); Taplin, M. E., Shuster, G. J., Frantz, M. E., Spooner, A. E., Ogata, G. K., Keer, H. N., and Balk, S. P., “Mutation of the androgen-receptor gene in metastatic androgen-independent prostate cancer,” New Eng. J. Med., 332:1393-1398, (1995); Gottlieb, B., Beitel, L. K., and Trifiro, M., “Variable Expressivity and Mutation Databases: The Androgen Receptor Gene Mutations Database,” Human Mutation, 17:382-388, (2001)) which are the most common forms of malignancy in men, and androgen insensitivity syndromes (Gottlieb, B., Pinsky, L., Beitel, L. K., and Trifiro, M., “Androgen Insensitivity,” American J. Medical Genetics (Semin. Med. Genet.), 89, 210-217, (1999)), but its role is not yet fully understood.

The androgen receptor is a member of a family of receptors, the nuclear receptors (NR's). Nuclear receptors represent a superfamily of proteins that specifically bind a physiologically relevant small molecule, such as a hormone. Generally, the binding occurs with high affinity so that apparent K_{d}'s are commonly in the 0.01-20 nM range, depending on the nuclear receptor/ligand pair. Nuclear receptors modulate, i.e., enhance or repress, the transcription of DNA, although they may have other, transcription independent, actions. Unlike integral membrane receptors and membrane associated receptors, the nuclear receptors reside in either the cytoplasm or nucleus of eukaryotic cells. As a result of a molecule binding to a nuclear receptor, the nuclear receptor changes the ability of a cell to transcribe DNA. Specifically, the nuclear receptors, and in particular AR, regulate gene expression by interacting with specific DNA sequences of target genes (see, e.g., Yamamoto, K., “Steroid receptors regulated transcription of specific genes and gene network,” Ann. Rev. Genetics, 19, 209, (1985); and Beato, M., “Gene regulation by steroid hormones”, Cell, 56:335-344, (1989)). Thus, the nuclear receptors comprise a class of intracellular, soluble, ligand-regulated transcription factors.

Nuclear receptors control nearly all critical biological processes from development to metabolism (see, e.g., Gronemeyer, H., and Laudet, V., The Nuclear Receptor Facts Book, Academic Press, London (2002); Altucci, L., and Gronemeyer, H., “Nuclear receptors in cell life and death,” Trends in Endocrinology and Metabolism, 12:460-468, (2001)). Identified in the human genome are forty-eight nuclear receptors, but for around half of these proteins, no function is known. Nuclear receptors are classified according to the hormone that they bind, and include receptors for glucocorticoids (GR's), androgens (AR's), mineralocorticoids (MR's), progestins (PR's), estrogens (ER's), thyroid hormones (TR's), vitamin D (VDR's), and retinoids (RAR's and RXR's). The so called “orphan receptors” are also part of the nuclear receptor superfamily, because they are structurally homologous to the classic nuclear receptors, such as steroid and thyroid receptors, and were originally named as such because they had no known ligand (see, Guigere, V., Yang, N., Segui, P., and Evans, R., “Identification of a new class of steroid hormone receptors,” Nature, 331:91, (1988)). However, it is now the case that ligands have been discovered for a number of orphan receptors (see, e.g., Gronemeyer and Laudet, The Nuclear Receptor Facts Book, Academic Press, (2002), at page 3).

All nuclear receptors with defined functions, such as the estrogen receptor and the glucocorticoid receptor, are major targets for pharmaceuticals (Tenbaum, S., and Baniahmad, A., “Nuclear Hormone Receptors: Structure, Function and Involvement in Disease,” Int. J. Biochem. and Cell Biol., 29:1325-1341, (1997)). Of those with known ligands, there are three principal categories (see, e.g., Evans, R. M., “The steroid and thyroid hormone receptor superfamily,” Science, 240:889, (1988); Keller, E. T., Ershler, W. B., and Chang, C., “The androgen receptor: a mediator of diverse responses,” Frontiers in Bioscience, 1:5971, (1996); Gronemeyer and Laudet, The Nuclear Receptor Facts Book, (2002); and Altucci and Gronemeyer, Trends in Endocrinology and Metabolism, 12:460-468, (2001)): 1) steroid receptors (comprising the glucocorticoid, progestin, mineralocorticoid, androgen, and estrogen receptors); 2) steroid derivatives (for example, vitamin D3); and 3) non-steroids (comprising the thyroid, retinoid, and prostaglandin receptors). Relationships between the various categories are shown in FIG. 1.

The medical importance of nuclear receptors is significant. They have been implicated in breast cancer, prostate cancer, cardiac arrhythmia, infertility, osteoporosis, hyperthyroidism, hypercholesterolemia, obesity and other conditions. In particular, one nuclear receptor, the androgen receptor (AR) is a key factor in mediating a wide variety of physiological processes, including regulation of male development, and the behavior of the prostate (see, e.g., Keller, et al., Frontiers in Bioscience, 1:5971, (1996)). AR binds hormones, referred to as “androgens”, which include male sex steroids, such as testosterones, including 5α-dihydrotestosterone (DHT). In normal physiological action, AR plays a role in embryogenesis, homeostasis, the development of sexual organs, reproduction, and cell growth and death in many classes of cells. However, in pathological conditions, AR is implicated in prostate cancers, androgen insensitivity syndromes (AIS), and spinal and bulbal muscular atrophy (Kennedy's disease).

Architecture of Nuclear Receptors

Nuclear receptors are composed of several structural domains (see, e.g., Jenster, G., van der Korput, H. A., van Vroonhoven, C., van der Kwast T. H., Trapman, J., and Brinkmann, A. O., “Domains of the Human Androgen Receptor Involved in Steroid Binding, Transcriptional Activation, and Subcellular Localization,” Molecular Endocrinology, 5, 1396-1404, (1991), and Jenster, G., van der Korput, H. A., van Vroonhoven, C., Trapman, J., and Brinkmann A. O., “Identification of two transcription activation units in the N-terminal domain of the androgen receptor amino-terminal domain,” J. Biol. Chem., 271, 7341-7346 (1995)), but the mapping of a particular function to a structural domain is usually quite difficult since individual domains not only interact with each other but with as many as 10-30 protein partners (see, e.g., Weatherman, R. V., Fletterick, R. J., and Scanlan, T. S., “Nuclear-receptor Ligands and Ligand-binding Domains,” Ann. Rev. Biochem., 68:559-581, (1999)). The various domains are shown schematically in FIG. 2.

The modularity of the nuclear receptor superfamily permits different domains of each protein to separately accomplish different functions, although the domains can influence each other. FIG. 2 provides a schematic representation of family member structures, indicating functions of the various domains. An area of the C-terminal domain, labeled “F”, in combination with various other parts of the AR sequence, is able to carry out a number of different functions. For example: DNA binding is achieved with a DNA binding domain on the N-terminal (region C in FIG. 2); hormone binding is achieved with a ligand binding domain overlapping regions D and E; homodimerization of androgen receptor molecules utilizes regions from C and E; nuclear localization signal (“LS”) utilizes two segments from regions C and D; transactivation function arises from two domains, AF1 and AF2, found on the N-terminal domain, and region E, respectively; and a domain for repression and silencing is found overlapping regions D and E. Overall sequence conservation between nuclear receptors varies between different families of receptors; however, sequence conservation between functional regions, or modules, of the receptors is high.

Generally, proteins of the nuclear receptor superfamily are considered to contain three principal modular functional domains: a variable N-terminal transcriptional activation domain (NTD); a highly conserved central DNA binding domain (DBD); and a less conserved C-terminal ligand binding domain (LBD). The LBD of nuclear receptors represents a hormone/ligand-dependent molecular switch, and recognizes a variety of compounds diverse in their size, shape and chemical properties. For example, the androgen hormones (“androgens”) exert their physiological effects by binding to the androgen receptor LBD. Binding of a hormone to a nuclear receptor's LBD also changes its ability to modulate transcription of DNA.

Most members of the nuclear receptor superfamily, including some orphan receptors, possess at least two transcription activation subdomains, one of which (AF-1) is constitutive and resides in the amino terminal domain, and the other of which (AF-2, also referred as TAU 4) resides in the ligand-binding domain and has activity that is regulated by binding of an agonist ligand. The function of AF-2 requires an activation domain (also called the transactivation domain) which is highly conserved among the receptor superfamily. The activity of AF-1 is regulated by growth factors and is generally believed to be activated in a ligand-independent manner, while AF-2 activity (“transcriptional activity”) is responsive to ligand binding. The binding of agonists triggers transcriptional activity whereas the binding of antagonists does not.

As well as ligands such as hormones, nuclear receptors also bind proteins, such as chaperone complexes, corepressors, or coactivators, that are critical to receptor function. In particular, ligand-dependent activation of transcription by nuclear receptors is mediated by interactions with coactivators. Some receptor agonists promote coactivator binding, and some antagonists block coactivator binding. Thus hormone binding by a nuclear receptor can increase or decrease binding affinity to these coactivator proteins, and can influence or mediate the multiple actions of the nuclear receptors on transcription.

Amino Terminal Domain

The amino terminal domain (or N-terminal domain, “NTD”) is the least conserved of the three domains and varies markedly in size among nuclear receptor superfamily members (for example, this domain contains 24 amino acids in the VDR and 550 amino acids in AR). This domain is involved in transcriptional activation and in some cases its uniqueness may dictate selective receptor-DNA binding and activation of target genes by specific receptor isoforms. This domain can display synergistic and antagonistic interactions with the domains of the LBD. For example, studies with mutated and/or deleted receptors show positive cooperativity of the amino and carboxy terminal domains (CTD's). In some cases, deletion of either of these domains will abolish the receptor's transcriptional activation functions. The NTD is required for activation of AR. The NTD of AR contains the FXXLF (SEQ ID NO: 1) motif, as well as the WXXLF motif, both of which have been shown, by mutagenesis, to participate in transactional activation.

DNA-Binding Domain

The DBD is the most conserved structure in the nuclear receptor superfamily. It usually contains about 70 amino acids that fold into two zinc finger motifs, wherein a zinc ion coordinates four cysteines. DBD's typically contain two perpendicularly oriented α-helices that extend from the base of the first and second zinc fingers. The two zinc fingers function in concert along with non-zinc finger residues to direct nuclear receptors to specific target sites on DNA and to align receptor homodimer or heterodimer interfaces. Various amino acids in DBD influence spacing between two half-sites (usually comprised of six nucleotides) for receptor dimer binding. For example, GR subfamily and ER homodimers bind to half-sites spaced by three nucleotides and oriented as palindromes. The optimal spacings facilitate cooperative interactions between DBD's, and D box residues are part of the dimerization interface. Other regions of the DBD facilitate DNA-protein and protein-protein interactions required for RXR homodimerization and heterodimerization on direct repeat elements.

The LBD may influence the DNA binding of the DBD, and such an influence can also be regulated by ligand binding. For example, TR ligand binding influences the degree to which a TR binds to DNA as a monomer or dimer. Such dimerization also depends on the spacing and orientation of the DNA half sites.

The nuclear receptor superfamily has also been subdivided into two subfamilies on the basis of DBD structures, interactions with heat shock proteins (hsp), and ability to form heterodimers: 1) GR (GR, AR, MR and PR), and 2) TR (TR, VDR, RAR, RXR, and most orphan receptors). GR subgroup members are tightly bound by chaperones in the absence of ligand, usually dimerize following ligand binding and dissociation of chaperone, and show homology in the DNA half sites to which they bind. These half sites also tend to be arranged as palindromes. TR subgroup members tend to be bound to DNA or other chromatin molecules when unliganded, can bind to DNA as monomers and dimers, but tend to form heterodimers, bind DNA elements with a variety of orientations and spacings of the half sites, and also show homology with respect to the nucleotide sequences of the half sites.

Carboxy-Terminal Subdomain

The carboxy-terminal activation subdomain is in close three dimensional proximity in the LBD to the ligand, so as to allow for ligands bound to the LBD to coordinate (or interact) with amino acid(s) in the activation subdomain.

Ligand Binding Domain

The LBD is the second most highly conserved domain in the nuclear receptor family. Whereas integrity of several different LBD sub-domains is important for ligand binding, truncated molecules containing only the LBD retain normal ligand-binding activity. The LBD also participates in other functions, including dimerization, nuclear translocation and transcriptional activation, as described herein. Importantly, this domain binds the ligand and undergoes ligand-induced conformational changes. It has been found that the LBD comprises 11-13 α-helices, labeled H-1, through H-12.

Most LBD's contain an activation domain. Some mutations in this domain abolish AF-2 function, but leave ligand binding and other functions unaffected. Ligand binding allows the activation domain to serve as an interaction site for essential co-activator proteins that stimulate (or in some cases, inhibit) transcription.

Recent structural studies suggest that, in some NR's, ligands regulate transcriptional activity by altering the structure of the LBD. For example, comparison of the structure of the unliganded human retinoid X receptor α LBD (RXRα) (Bourguet, et al., Nature, 375:377-82, (1995)) with the structures of the liganded LBD's of the human retinoic acid receptor γ (RARγ) (Renaud, et al., Nature, 378:681-689, (1995) and Wurtz, J.-M., Bourguet, W., Renaud, J.-P., Vivat, V., Chambon, P., Moras, D., and Gronemeyer, H., “A canonical structure for the ligand-binding domain of nuclear receptors,” Nature Struct. Biol., 3, 87-94, (1996)), the thyroid hormone receptor α (TRα) (Wagner, et al., Nature, 378:690-697, (1995)), the progesterone receptor (PR) (Williams, et al., Nature, 393:392-395, (1998)), and the ERα (Brzozowski, et al., Nature, 389:753-758, (1997); Tanenbaum, et al., Proc. Natl. Acad. Sci. USA, 95:5998-6003, (1998)) suggests that an agonist-induced conformational change involving the repositioning of helix 12, the most C-terminal helix of the LBD, is essential for transcriptional activity. Furthermore, because certain point mutations in helices 3, 5 and 12 abolish transcriptional activity but have no effect on ligand or DNA binding, these regions of the LBD have been predicted to form part of a recognition surface, created in the presence of agonist, for molecules that link the receptor to the general transcriptional machinery (see, e.g., Danielian, et al., EMBO J., 11:1025-33, (1992); Feng, et al., Science, 280:1747-9, (1998); Henttu, et al., Mol. Cell. Biol., 17:1832-9, (1997); Wrenn, et al., J. Biol. Chem., 268:24089-24098, (1993)).

Coactivators and Coactivator Binding

Biochemical and genetic approaches have led to the identification of several proteins that associate in a ligand-dependent manner with nuclear receptors (see, e.g., Horwitz, et al., Mol. Endocrinol., 10:1167-1177, (1996)). Such proteins include SRC-1/N-CoA1 (Onate, et al., Science, 270:1354-1357, (1995)), GRIP1/TIF2/SRC-2 (Hong, et al., Proc. Natl. Acad. Sci. USA, 93(10):4948-4952, (1996); and Voegel, et al., EMBO J., 15:3667-3675 (1996)), p/CIP/RAC3/ACTR/AIB1/SRC-3 (Anzick, et al., Science, 277:965-968(1997), Chen, et al., Cell, 90(3):569-80, (1997); Li, et al., Proc. Natl. Acad. Sci. USA, 94:8479-84, (1997); and Torchia, et al., Nature, 387:677-684, (1997)), and CBP/p300 (Hanstein, et al., Proc. Natl. Acad. Sci. USA, 93:11540-11545, (1996)). These proteins have been classified as transcriptional coactivators because they enhance ligand-dependent transcriptional activation by a number of NR's (Glass, et al., Curr. Opin. Cell Biol., 9:222-32 (1997); Torchia, et al., Nature, 387:677-684, (1997)).

The observation of partial hormone resistance in mice with a disrupted SRC-1 gene (Xu, et al., Science, 279:1922-1925, (1998)) provided compelling evidence that coactivators are required for NR function in vivo. Consistent with its proposed role in AF-2 directed transcriptional activation, SRC-1 possesses histone acetylase activity and the ability to interact not only with agonist-bound receptors but also with other coactivators and several general transcription factors (Kamei, et al., Cell, 85(3):403-14, (1996); Onate, et al., cited hereinabove; Spencer, et al., Nature, 389:194-8, (1997); Takeshita, et al., Endocrinology, 137:3594-7, (1996)). SRC-1 and GRIP1 also bind to the agonist-bound LBD's of both the human TRβ and human ERα using a putative coactivator binding site (Feng, et al., cited hereinabove).

The structural and functional nature of the site to which coactivators bind has only recently been defined, Apriletti, et al., U.S. Patent Application Publication No. 2002/0061539 A1, published May 23, 2002, the disclosure of which is incorporated herein by reference in its entirety. It has been shown that the NR LBD has a surface exposed hydrophobic cleft (formed by helices 3, 4, 5, and 12) that interacts with short hydrophobic motifs of co-activator partners (see, Feng, W., Ribeiro, R. C. J., Wagner, R. L., Nguyen, H., Apriletti, J. W., Fletterick, R. J., Baxter, J. D., Kushner, P. J., and West, B. L., “Hormone-Dependent Co-activator Binding to a Hydrophobic Cleft on Nuclear Receptors” Science, 280:1747-1749, (1998); Darimont, B. D., Wagner, R. L., Apriletti, J. W., Stallcup, M. R., Kushner, P. J., Baxter, J. D., Fletterick, R. J., Yamamoto, K. R., “Structure and specificity of nuclear receptor-coactivator interactions,” Genes and Development, 1,12(21), 3343-56, (1998); and Coultard, V. H., Matsuda, S., and Heery, D. M., “An extended LXXLL motif sequence determines the nuclear receptor binding specificity of TRAP220”, J. Biol. Chem., 278(13):10942-51, (2003)). In nuclear receptors other than the steroid receptors, corepressors and coactivators are known to compete for this hydrophobic surface cleft and bind to form an enlarged protein complex for regulating transcription. In general, similar modes of coactivator binding have been found in other members of the nuclear receptor family, including the TR, ER and PPARγ receptors.

The p160 Steroid Receptor Coactivator Family

The p160 Steroid Receptor Coactivator (SRC) gene family contains three homologous members: SRC-1 (NcoA-1), SRC-2 (GRIP1, TIF2, or NcoA-2) and SRC-3 (p/CIP, RAC3, ACTR, AIB1, or TRAM-1) which serve as transcriptional coactivators for nuclear receptors and certain other transcription factors. GRIP1 was identified through its interactions with LBD's of the glucocorticoid receptor (GR) and estrogen receptor (ER). The SRC proteins are about 160 kDa in size and have an overall sequence similarity of 50-55% and sequence identity of 43-48% between the three members.

Many nuclear receptor coactivators contain one or more conserved regions, called “nuclear receptor boxes” (“NR-boxes”), that are responsible for interaction with hormone-bound nuclear receptors. For example, the relatively conserved central region of the SRC family members contains three motifs that have the sequence LXXLL (SEQ ID NO: 2) (where L is leucine and X is any amino acid). The NR-boxes form short amphipatic helices which bind to the LBD hydrophobic groove, in the case of SRC members via their Leu residues.

Mutagenesis studies indicate that the affinity of coactivators for NR LBD's is determined principally, if not exclusively, by these NR boxes (Ding, et al., Mol. Endocrinol, 12:302-313, (1998)); Heery, et al., Nature, 387:733-736, (1997); Le Douarin, et al., EMBO J., 15:6701-15, (1996); Torchia, et al., et al., Nature, 387:677-684, (1997)). Each of the p160 coactivators contains several NR boxes. The NR boxes within SRC-1, GRIP1 and TIF2 have been demonstrated to recognize different NR's with different affinities (Ding, et al., Mol. Endocrinol, 12:302-313, (1998); Kalkhoven, et al., EMBO J., 17:232-43 (1998); Voegel, et al., EMBO J., 17:507-19, (1998)), but the reasons for these binding preferences are unknown. Structural studies of the complex between TRβ and the GRIP1 NR Box 2 peptide and biochemical studies of GRIP1 binding to TRβ and GR have been described (Darimont, et al., “Structure and specificity of nuclear receptor-coactivator interactions,” Genes Dev., 12:3343-3356, (1998)). The PPARγ/SRC-1 peptide complex is described in Nolte, et al., Nature, 395:137-143, (1998).

Members of the p160 family of coactivators, such as SRC-1, GRIP1/TIF2/SRC-2, and p/CIP/RAC3/ACTR/AIB1/SRC-3, as well as other coactivators recognize agonist-bound NR LBD's through the short signature sequence motif, LXXLL (Ding, et al., Mol. Endocrinol, 12:302-313, (1998); Heery, et al., Nature, 387:733-736, (1997); Le Douarin, et al., EMBO J., 15:6701-15, (1996); Torchia, et al., cited hereinabove). SRC-2 box 3 is the best-known AR-interacting partner, and binds AR LBD with micromolar affinity.

Three commonly found NR-boxes have been labeled NR Boxes 1, 2, and 3. Alignment of the sequences of a number of coactivators, showing the presence of these boxes, is shown in FIG. 3.

The Androgen Receptor

The androgen receptor has wide tissue distribution and can be demonstrated by immunohistochemistry in several tissues e.g., prostate (Zhuang, Y. H., Blauer, M., Pekki, A., et al., “Subcellular location of androgen receptor in rat prostate, seminal vesicle and human osteosarcoma MG-63”, J. Steroid Biochem. and Molec. Biol., 41:693-696, (1992)), skin (see, e.g., Blauer, M., Vaalasti, A., Pauli, S-L., et al., “Location of androgen receptor in human skin”, J. Investigat. Derm., 97:264-268, (1991)) and oral mucosa. The presence of the androgen receptor can also be demonstrated in a diverse range of human tumours, e.g., osteosarcoma (Zhuang, et al., J. Steroid Biochem. and Molec. Biol., 41:693-696, (1992)). In prostatic carcinoma, androgen receptor expression may be of clinical relevance (see, e.g., Demura, T., Kuzumaki, N., Oda, A., et al., “Establishment of monoclonal antibody to human androgen receptor and its clinical application for prostatic cancer”, Am. J. Clinical Oncol., 11(2):S23-S26, (1988)). Mutation of the gene encoding androgen receptor has been reported in prostatic carcinoma (Barrack, E. R., Newmark, J. R., Hardy, D. O., et al., “Androgen receptor gene mutations in human prostate cancer”, J. Cell Biochem., 16D:93, (1992)). Details of commercially available samples of androgen receptor can be found at www.novocastra.co.uk/data/hrerp/ar_p.pdf.

In essence, AR binds to DNA, upon androgen hormone binding, and then acts as a transcription factor that regulates the expression of from about 20 to hundreds of genes depending on the cell type (see, e.g., Keller, E. T., et al., Frontiers in Bioscience, 1: 5971, (1996); and, Beato, M., “Gene regulation by steroid hormones,” Cell, 56: 335-344, (1989)). However, the underlying mechanism is actually more complicated. It is understood that activation of androgen receptor (AR), initiated by binding of a hormone such as DHT to the androgen receptor ligand binding domain (LBD), changes the three dimensional structure of the LBD, and causes the androgen receptor to dissociate from chaperones in the cytoplasm and travel into the nucleus where the receptor binds response elements on DNA. This mechanism is effectively a kind of control mechanism that ensures that androgen receptors are kept away from DNA molecules until they have been suitably activated.

The activation of transcription by AR is complex since it involves more conformational rearrangements and cofactor interactions than other hormone receptors. Phosphorylation and SUMOylation (modification by attachment to a small ubiquitin-like modifier) may also have critical regulatory roles that are just now being discovered (see, Poukka, H., Karvonen, U., Jänne, O. A., Palvimo, J. J., “Covalent modification of the androgen receptor by small ubiquitin-like modifier 1 (SUMO-1),” Proc. Nat. Acad. Sci. USA, 97, 14145-14150 (2000); Gioeli, D., Ficarro, S. B., Kwiek, J. J., Aaronson, D., Hancock, M., Catling, A. D., White, F. M., Christian, R. E., Settlage R. E., Shabanowitz, J., Hunt, D. F., Weber, M. J., “Androgen Receptor Phosphorylation: Regulation and Identification of the Phosphorylation Sites,” J. Biol. Chem., 277:29304-29314, (2002)).

Activation of AR is a two-step process. The first step is androgen binding to the ligand-binding domain (LBD) (see, e.g., Weatherman, et al., Ann. Rev. Biochem., 68, 559-581, (1999); Wurtz, J.-M., et al., Bourguet, W., Renaud, J.-P., Vivat, V., Chambon, P., Moras, D., and Gronemeyer, H., “A canonical structure for the ligand-binding domain of nuclear receptors,” Nat. Struct. Biol., 3:87-94, (1996); Moras, D., and Gronemeyer, H., “The nuclear receptor ligand binding domain: structure and function,” Curr. Op. Cell Biol., 10, 384-391, (1998); and Bourguet, W., Germain, P., & Gronemeyer, H., “Nuclear receptor ligand-binding domains: 3D structures, molecular interactions and pharmacological implications,” Trends in Pharmaceutical Scences, 21, 381-388 (2000)), which induces conformational changes in the receptor that promote dissociation of regulatory proteins and association with critical binding partners (see, e.g., Weatherman, et al., Ann. Rev. Biochem., 68:559-581, (1999)). The second step is the binding of the AR to such binding partners. The second step is obligatory for AR activation of transcription. In addition, coactivators mediate the transcriptional activity of AR.

AR is unusual among the family of nuclear receptors because it requires an interaction between the N-terminal and ligand binding domains to achieve its activated conformation (see, e.g., He, B., Kemppainen, J. A., and Wilson E. M,. “Activation Function 2 in the Human Androgen Receptor Ligand Binding Domain Mediates Interdomain Communication with the NH₂-terminal Domain,” J. Biol. Chem., 274 (52), 37219-37225 (1999); and He, B., Kemppainen, J. A., and Wilson E. M., “FXXLF and WXXLF Sequences Mediate the NH₂-terminal Interaction with the Ligand Binding Domain of the Androgen Receptor,” J. Biol. Chem., 275(30), 22986-22994, (2000)). This interaction involves the formation of a putative α-helical motif (with the motif sequence FXXLF) in the NTD, and its binding to a cognate receptor surface in the LBD (see, e.g., Feng, W., Ribeiro, R. C. J., Wagner, R. L., Nguyen, H., Apriletti, J. W., Fletterick, R. J., Baxter, J. D., Kushner, P. J., and West, B. L., “Hormone-Dependent Co-activator Binding to a Hydrophobic Cleft on Nuclear Receptors”, Science, 280, 1747-1749, (1998); Darimont, B. D., Wagner, R. L., Apriletti, J. W., Stallcup, M. R., Kushner, P. J., Baxter, J. D., Fletterick, R. J., Yamamoto, K. R,. “Structure and specificity of nuclear receptor-coactivator interactions”, Genes and Development, 1,12(21), 3343-56, (1998); and Coultard, V. H., Matsuda, S., and Heery, D. M., “An extended LXXLL motif sequence determines the nuclear receptor binding specificity of TRAP220”, J. Biol. Chem., 278(13):10942-51, (2003)).

AR Co-Activators and Co-Activator Binding

AR Associated Proteins (ARA's) are a class of AR-specific co-regulatory proteins, of which ARA70 (also known as ELE1α) was the first to be described. The ARA's further include the LBD interacting proteins ARA24, ARA54, ARA55 and ARA160 (also called TATA element modulatory factor, TMF). ARA70 is about one third of the size of GRIP-1 and has the motif FXXLF which has been implicated in transactivation functions. ARA70's putative role was elucidated in prostate cancer DU-145 cells (see Yeh, S., and Chang, C., “Cloning and characterization of a specific coactivator, ARA70, for the androgen receptor in human prostate cells,” PNAS, 93:5517-5521, (1996)). In these cells, which were transfected with ARA70, enhancement of functional activity of AR by testosterone and DHT was measured. ARA70 has only a weak effect on transcriptional activation of steroid receptors other than AR (see Yeh and Chang, PNAS, 93, 5517-5521, (1996)), and therefore it exhibits virtually no receptor promiscuity. Several lines of evidence implicate ARA70 in the acquired agonist activity of anti-androgens, and in making prostate cancer cells resistant to ablation and/or antiandrogen therapy, but hitherto its mode of action has not been fully understood.

The AR LBD binds the LXXLL sequences that are repeated several times in co-activators, but the interaction is only weak, in contrast to other nuclear receptors. However, AR preferentially interacts in a ligand dependent manner with two homologous motifs present in the NTD (FXXLF and, to a lesser degree, WXXLF (SEQ ID NO: 3)). ARA70 also contains FXXLF motifs and hence its cocrystallization with AR LBD is of high interest. Recently, the interaction of peptides having (F/W)XXL(F/W) (SEQ ID NO: 4) and FXXLY (SEQ ID NO: 5) motifs with AR was reported, using phage display techniques (see Hsu, C.-L., Chen, Y.-L., Yeh, S., Ting, H.-J., Hu, Y.-C., Lin, H., Wang, X., and Chang, C., J. Biol. Chem., 278:23691-23698, (2003)), but no structure of the binding interface was presented. Hsu, C.-L., et al., J. Biol. Chem., 278:23691-23698, (2003).

No crystal structure of the complete androgen receptor has been made available. One structure of the hAR LBD, bound to the synthetic ligand metribolone (R1881) has been described (see, Matias, P. M., Donner, P., Coelho, R., Thomaz, M., Peixoto, C., Macedo, S., Otto, N., Joschko, S., Scholz, P., Wegg, A., Basler, S., Schafer, M., Egner, U., Carrondo, M. A., “Structural Evidence for Ligand Specificity in the Binding Domain of the Human Androgen Receptor. Implications for Pathogenic Gene Mutations”, J. Biol. Chem., 275:26164-26171, (2000)). Another structure of the LBD of AR, and its mutant T877A, complexed with the natural agonist DHT, refined at 2.0 Å resolution, has also been presented (see, Sack, J. S., Kish, K. F., Wang, C., Attar, R. M., Kiefer, S. E., An, Y., Wu, G. Y., Scheffler, J. E., Salvati, M. E., Krystek Jr., S. R., Weinmann, R., Einspahr, H. M., “Crystallographic Structure of the Ligand-Binding Domains of the Androgen Receptor and its T877A Mutant Complexed with the Natural Agonist Dihydrotestosterone”, Proc. Nat. Acad. Sci. USA, 98, 4904-4909, (2001)). None of these structures has included a coactivator molecule bound to the coactivator binding site.

AR and Prostate Cancer

AR's critical role in male prostate cancer is well-documented (see, e.g., Tenbaum, S., and Baniahmad, A., Int. J. Biochem. and Cell Biol., 29, 1325-1341, (1997); Taplin, M. E., Shuster, G. J., Frantz, M. E., Spooner, A. E., Ogata, G. K., Keer, H. N., and Balk, S. P., “Mutation of the androgen-receptor gene in metastatic androgen-independent prostate cancer,” New Eng. J. Med., 332:1393-1398, (1995); and Gottlieb, B., Beitel, L. K., and Trifiro, M., “Variable Expressivity and Mutation Databases: The Androgen Receptor Gene Mutations Database,” Human Mutation, 17:382-388, (2001)). Consequently, current research in prostate cancer is aimed at finding new ways to inhibit AR function in pathological states, though none of this work has specifically addressed coactivator binding.

Mutations in the AR gene are thought to be responsible for prostate cancer, and androgen insensitivity syndrome (AIS). The NTD and LBD interaction required to activate AR is hormone dependent, and is disrupted by mutation in the receptor face of the LBD. Such disrupting mutations have been associated with androgen insensitivity syndrome in human patients (see, e.g., Gottlieb, B., Pinsky, L., Beitel, L. K., and Trifiro, M., “Androgen Insensitivity,” American J Medical Genetics (Semin. Med. Genet), 89, 210-217 (1999)).

Current treatment of prostate cancer is often with anti-testosterones, such as flutamide (cyproterone acetate), nilutamide, and bicalutamide (casodex), which suppress AR function. However, after 3-5 years of treatment with these agents, the treatment becomes less effective. In particular, prostate-specific antigen (PSA) levels are seen to rise in patients; the presence of such antigens indicates AR activation. The rise in malignant transcriptional activity has been attributed to AR being activated inappropriately. Recently, the endogenous coactivator, ARA70, a potent special activator of AR, has been implicated in anti-testosterone refractory prostate cancer (see Rahman, M. M., Miyamoto, H., Takatera, H., Yeh, Shuyuan, Altuwaijri, S., and Chang, C., J. Biol. Chem., 278:19619-19626, (2003)). Specifically, a dominant-negative ARA70 mutant was shown to inactivate ARA70, halting androgen-independent growth of LNCAP prostate tumor cells. Thus, dominant-negative ARA70, or RNA-interference-mediated silencing, of ARA70 reduces agonist activity and rescues the normal function of anti-androgens. However, the dominant negative mutant in question (“dARA70N”) did not itself bind to the AR coactivator binding site, leading to the postulate that the mutant inactivated the normal function of ARA70 through heteromer formation between the mutant and endogenous ARA70 (Rahman, et al., J. Biol. Chem., 278:19619-19626, (2003)).

A decade or more ago, the paucity of structural data on the LBD's and coactivator binding sites of nuclear receptors has meant that the development of synthetic ligands and coactivators that specifically bind to nuclear receptors has been largely guided by trial and error. Thus, new ligands specific for nuclear receptors were often discovered in the absence of information on the three dimensional structure of a nuclear receptor with a bound ligand. More recently, design of organic molecules that bind to the LBD has become possible, due to the availability of structural data on ligand binding. But, by contrast, methods for discovery of molecules that block coactivator binding directly at the coactivator binding site have remained elusive for many nuclear receptors—including the androgen receptor. Accordingly, before the present invention, researchers were essentially discovering nuclear receptor coactivators by probing in the dark and without the ability to visualize how the amino acid residues of a nuclear receptor held a coactivator in their grasp.

The discussion of the background to the invention herein is included to explain the context of the invention. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY OF THE INVENTION

The present invention relates to the identification and characterization of the coactivator binding site of nuclear receptors, thereby facilitating the design of compounds that bind to the coactivator binding site for the purpose of modulating nuclear receptor activity. The present invention relates in particular to the androgen receptor (AR), and also to other members of the steroid receptor family. The compounds that bind to the coactivator binding site include antagonists that modulate nuclear receptor activity, and can be receptor-, cell- and/or tissue-specific. In particular, the compounds designed or identified by the methods of the present invention modulate androgen receptor activity by affecting interactions between a coactivator and the coactivator binding site of the androgen receptor.

The naturally occurring AR coactivator ARA70 is implicated in the onset of insensitivity to the anti-androgen, flutamide, amongst prostate cancer patients undergoing treatment. Use of a coactivator mimic to block binding of ARA70 to the coactivator binding site has potential clinical value for treating androgen refractory prostate cancer. In particular, a mimic of ARA70 may be effective together with flutamide.

According to the methods of the present invention, it is deduced that, when AR preferentially binds ARA70, a structural reorganization of the coactivator binding site takes place that activates transcription, as illustrated by structures whose coordinates are presented in PDB files stored on CD-R herein. By contrast, the LBD's of some other known nuclear receptors such as ER and TR, do not bind ARA70, and do not undergo significant structural rearrangements upon coactivator binding, although they may undergo such changes upon binding a ligand such as a hormone.

The changes to the AR LBD that occur on and after binding a hormone alter the associations of the receptor with itself such as its N-terminal domain, or in dimer formation, and with other proteins, in particular coactivators. The structure of the cocrystals of the present invention reveal that coactivator partners interact with the AR LBD surface at the coactivator binding site. Such coactivators include p160 and ARA70, as well as the N-terminal domain of the AR itself.

The present invention also includes an isolated and purified protein complex comprising: an androgen receptor ligand binding domain; a ligand bound to the ligand binding domain; and a coactivator bound to a coactivator binding site of the ligand binding domain. The present invention further includes methods for making the same. Such complexes can be formed in solution from a ligand-bound AR LBD and peptides of about 15 amino acid residues. Such peptides can be found by testing their binding to the AR coactivator binding site using either an isolated form of the AR LBD, or the complete receptor. The peptides are preferably part of a bacterial phage carrying peptides of 15 amino acids displayed on the phage surface. Approximately 109 peptides with stochastic amino acid sequences are present in the phage libraries. These peptides in some cases contain amino acid sequences that likely represent the core binding motifs from the interaction domains of the N terminal domain of the androgen receptor itself, GRIP-1, and ARA 70. The peptides interact differently with the hormone binding domain of the androgen receptor depending on the specific sequence found in the principal contact amino acids of the peptide. It is to be understood that the coactivator bound to the coactivator binding site may be a fragment of a known coactivator.

The present invention further comprises a purified and crystallized form of the ligand binding domain of the human androgen receptor, bound to a ligand and a coactivator. The present invention also includes protein cocrystals of an androgen receptor with an androgen bound to the LBD and another molecule bound to the coactivator binding site, and to methods for making the same. The present invention further includes crystals of AR bound to a peptide whose sequence comprises a portion of a sequence of a coactivator such as ARA70, and methods of obtaining the same. Specifically, the present invention provides cocrystals of the ligand binding domain of human AR with peptides whose sequences derive in part from co-regulatory proteins of AR such as ARA70, and GRIP1 box 1, GRIP1 box2, and GRIP1 box3 peptides, as well as the N-terminal domain of AR. The cocrystals also provide information regarding the ligand:nuclear receptor and coactivator:nuclear receptor interactions, as well the structure of molecules bound thereto. Thus, the cocrystals provide means to obtain atomic modeling information of the specific amino acid residues that form the LBD and the coactivator binding site, and their constituent atoms, and therefore the key functional groups that interact with molecules that bind to the sites. These crystal structures give rise to an understanding of how AR functions in physiological and pathological conditions, thereby leading to ways of developing molecules that would similarly bind to the coactivator binding site. Specifically, the crystal structures of AR complexes of the present invention provide a determination of receptor critical binding sites that underpin coactivator binding, thereby permitting design and development of anti-prostatic cancer drugs.

The present invention still further includes use of the structural information derived from such crystals to design ARA70 inhibitors, and for the use of such inhibitors in treating AR-dependent prostate cancers. New therapeutic treatments of AR-dependent prostate cancers may be developed that involve preventing essential conformational rearrangements of AR, or blocking coactivator binding. By blocking interactions between ARA70 and AR, anti-ARA70 binding therapeutics lower AR activity, thereby lowering PSA levels. Thus, the process of uncontrolled cell division can be disrupted.

Methods to mimic a coactivator such as the ARA70 molecule with a peptide or small organic molecule that binds more tightly than ARA 70 are provided herein to create a selective blocking agent for the association of ARA70 and androgen receptor.

Accordingly, the present invention further provides methods for identifying and designing molecules that modulate the activity of a nuclear receptor by using an atomic model of an androgen receptor to which is bound a ligand and a coactivator. The method involves modeling test compounds that fit spatially into a nuclear receptor coactivator binding site using an atomic structural model comprising an androgen receptor coactivator binding site or portion thereof to which is bound a ligand and a coactivator, screening the test compounds in an assay, such as a biological assay, characterized by binding of a test compound to the nuclear receptor coactivator binding site, and identifying a test compound that modulates nuclear receptor activity.

The invention further includes a method for identifying an antagonist of coactivator binding to a nuclear receptor. The method comprises providing the atomic coordinates comprising a nuclear receptor coactivator binding site or portion thereof to a computerized modeling system; modeling compounds which fit spatially into the nuclear receptor coactivator binding site; and identifying in an assay, for example a biological assay, for nuclear receptor activity a compound that increases or decreases activity of the nuclear receptor through binding the coactivator binding site.

Also provided is a method of identifying a compound that selectively modulates the activity of one type of nuclear receptor compared to other nuclear receptors. The method is exemplified by modeling test compounds that fit spatially and preferentially into an atomic structural model of the androgen receptor coactivator binding site, selecting a compound that interacts with one or more residues of the coactivator binding site that is unique to the androgen receptor site, and identifying in an assay, for example a biological assay, for coactivator binding activity a compound that selectively binds to the androgen receptor coactivator binding site compared to other nuclear receptors. The unique features involved in receptor-selective coactivator binding can be identified by comparing atomic models of different nuclear receptors or isoforms of the same type of receptor.

The present invention finds use in the selection and characterization of peptide, peptidomimetic, as well as other compounds, such as small organic molecules, identified by the methods of the invention, particularly new compounds useful in treating nuclear receptor-based disorders, in particular steroid receptor-based disorders, and more specifically androgen receptor-based disorders.

The methods of the present invention may be further used to design a molecule—for example a small organic molecule—that blocks AR coactivator binding, and that could therefore function as a drug.

Peptides comprising motifs found within the sequences of the bound coactivators have been shown to mimic the full size coregulators in the AR LBD:coactivator interface, partly recapitulating the associations found for the full length coactivators. Accordingly, one aspect of the present invention is the synthesis of constrained tighter binding peptide analogs of the FXXLF motif of ARA70. Such peptides are preferably able to be transported into a prostate cancer cell nucleus to block the association between ARA70 and AR therein, and thereby diminish AR transcriptional activation of prostate specific antigens. Methods for facilitating transport of such peptides include the use of fused carrier peptides, as would be understood by one of ordinary skill in the art. For example, peptides of the present invention could be fused to a peptide from a known virus, as described in, e.g., Yang, Y., Ma, J., Song, Z., Wu, M., “HIV-1 TAT-mediated protein transduction and subcellular localization using novel expression vectors”, FEBS Lett., 532(1-2):36-44, (2002).

The invention also includes methods for identifying molecular determinants of the interaction between AR and its physiological coactivators. Such molecular determinants include, but are not limited to, key residues within the coactivator binding sites of nuclear receptors. The methods involve examining the surface of a nuclear receptor of interest to identify residues that modulate ligand and/or coactivator binding. The residues can be identified by homology to the key residues within the LBD and the coactivator binding site of human AR described herein. Overlays and superpositioning with a three dimensional model of a nuclear receptor's coactivator binding site, and/or a portion thereof, also can be used for this purpose. Additionally, alignment and/or modeling can be used as a guide for the placement of mutations on the surface of the coactivator binding site of a nuclear receptor in order to characterize the nature of the site in different physiological contexts.

According to the present invention, a binding cleft for the coactivator ARA70, and mimics thereof, in the coactivator binding site on the hormone binding domain of the human androgen receptor is elucidated through methods of X-ray crystallography. The structure of such a binding cleft, in conjunction with a bound coactivator or coactivator mimic, also leads to characterization of the binding interface between AR and ARA70. In particular, it is found that the FXXLF motif found in ARA70 binds differently from previously known coactivators in ways that could not have been appreciated without the crystal structures disclosed herein.

The understanding of AR binding to a coactivator such as ARA70 that has been obtained from the present invention leads to the surprising deduction that a molecule, such as a small organic molecule, with only two points of binding is sufficient to inhibit ARA70 binding.

Also provided is a method of modulating the activity of a nuclear receptor. The method can be in vitro or in vivo. The method comprises administering in vitro or in vivo a sufficient amount of a compound that binds to the coactivator binding site and acts as an antagonist of binding to natural coactivators. Preferred compounds bind to the site with greater affinity than coactivators found in a cell of interest. In particular, the present invention provides a method of modulating the activity of the androgen receptor, comprising administering in vitro or in vivo a sufficient amount of a compound that acts as an antagonist of coactivator binding to the androgen receptor.

Also provided is a machine-readable data storage medium encoded with information for constructing and manipulating an atomic model comprising the coactivator binding site of the androgen receptor or portions thereof. The medium comprises a data storage material encoded with machine readable data which, when using a machine programmed with instructions for using said data, is capable of displaying a graphical three-dimensional representation of a molecule of the androgen receptor coactivator binding site, with or without another molecule complexed thereto.

The three dimensional structure of the AR LBD in its associations with peptides consisting of portions of the sequence of a natural coactivator proteins such as GRIP-1, and ARA70, as well as the N-terminal domain of the androgen receptor itself, reflect the association of the receptor with such known coactivators. GRIP-1 and ARA70 are important activators of the ligand binding domain of the androgen receptor under differing physiological conditions and in different cell types. These structures can be compared with an independently determined structure of the hormone binding domain of the androgen receptor with DHT bound and with the structure of the actual ARA70 peptide.

The form of binding to the coactivator binding site of AR may vary for different genetic encodings. For example, the coactivator binding site may bind to a coactivator such as ARA70, to a corepressor, as well as to the N-terminal domain. Accordingly, it is within the scope of the present invention to elucidate and design molecules that bind to coactivator binding sites and inhibit a number of different types of coactivators.

The present invention further provides methods of comparison of the binding of the FXXLF motif found in ARA70 and the LXXLL binding of a conventional AR coactivator such as GRIP-1. Such comparisons illustrate that, although many receptor side-chains are similarly positioned in the two binding situations, unexpectedly some, such as Met 734, adopt different conformations. The concerted movements of the several side chains create a binding cavity for the ARA70 FXXLF motif that differs from other coactivators and is thus the target for small molecule pharmaceutical design.

By visualizing crystal structures of AR bound to a number of coactivators, according to the present invention, it is possible to deduce determinants of specificity of binding. In particular, it is consistent with the methods of the present invention that the coactivator, or coactivator mimic, can be the N-Terminal Domain of AR itself, or some portion of the sequence thereof, preferably the FXXLF or WXXLF motifs contained therein.

The methods of the present invention will usually be applicable to other nuclear receptors, as discussed herein, in particular, to patterns of nuclear receptor activation, structure, and modulation that have emerged as a consequence of determining the three dimensional structures of the LBD and coactivator binding sites of such nuclear receptors with different ligands bound, for example the three dimensional structures or crystallized protein structures of the ligand binding domains of ligand-activated nuclear receptors such as members of the steroid hormone receptor family.

The present invention, particularly the computational methods presented herein, can be used to design coactivators, or inhibitors of coactivator binding, for a variety of nuclear receptors, such as receptors for glucocorticoids (GR's), androgens (AR's), mineralocorticolds (MR's), progestins (PR's), estrogens (ER's), thyroid hormones (TR's), vitamin D (VDR's), retinoid (RAR's and RXR's) and peroxisomal proliferators (PPAR's). The present invention is preferably applicable to members of the steroid receptor family, i.e., the glucocorticoids (GR's), androgens (AR's), mineralocorticolds (MR's), progestins (PR's), and estrogens (ER's). The present invention is even more preferably applied to the androgen receptor. It is further envisaged that the information obtained from the structures of the present invention may be further utilized with the structurally homologous glucocorticoid receptor (GR).

The present invention can also be applied to the “orphan receptors”, because they are structurally homologous in terms of modular domains and primary structure to classic nuclear receptors, such as steroid and thyroid receptors. The amino acid homologies of orphan receptors with other nuclear receptors ranges from very low (<15%) to in the range of 35% when compared to rat RARα and human TRβ receptors, for example. In addition, as is revealed by the X-ray crystallographic structure of the AR and structural analysis disclosed herein, the overall folding of any liganded member of the nuclear receptor superfamily is likely to be similar. Although very few ligands for orphan receptors have been identified, one of ordinary skill in the art will be able to apply the methods of the present invention to the design and use of such ligands, as their overall structural modular motif will be similar to other nuclear receptors described herein.

### BRIEF DESCRIPTION OF THE DRAWINGS

FIG. 1 shows relationships between various members of the nuclear receptor family.

FIG. 2 shows a schematic representation of the functional domains of the Androgen Receptor. AR consists of a highly conserved DNA binding domain (DBD), a variable N-terminal domain (NTD) and a conserved C-terminus (D, E and F regions). The D-region (hinge region) links other domains to phosphorylation, targeting or other protein partners. The DBD binds to specific sequence response elements. The N-Terminus harbors trans-activation function (AF1). The C-Terminus contains multiple hormone-dependent functions including hormone binding, trans-activation (AF2), silencing, dimerization and nuclear localization (see, e.g., Jenster, G., van der Korput, H. A., van Vroonhoven, C., van der Kwast T. H., Trapman, J., and Brinkmann A. O., “Domains of the Human Androgen Receptor Involved in Steroid Binding, Transcriptional Activation, and Subcellular Localization”, Molecular Endocrinology, 5, 1396-1404, (1991)).

FIG. 3 shows sequence alignment of amino acid residues of members of the p160 coactivator family. Single amino acid designations are used. Members of the p160 coactivator family interact with nuclear receptors through conserved (SEQ ID NO: 1) LXXLL motifs.

FIG. 4 shows alignment of amino acid sequences (single letter amino acid designations) containing residues that form the coactivator binding sites of several nuclear receptors: human and recombinant thyroid hormones, hTRβ (SEQ ID NOs: 5 and 6) and rTRα (SEQ ID NOs: 7 and 8); retinoids, hRARγ (SEQ ID NOs: 9 and 10) and hRXRα (SEQ ID NOs: 11 and 12)); peroxisome, hPPARγ, (SEQ ID NOs: 13 and 14); vitamin D, hVDR (SEQ ID NOs: 15 and 16); estrogen, hERα (SEQ ID NOs: 17 and 18); glucocorticoid, hGR (SEQ ID NOs: 19 and 20); progestin, hPR (SEQ ID NOs: 21 and 22); mineralocorticoid, hMR (SEQ ID NOs: 23 and 24); and androgen, hAR (SEQ ID NOs: 25 and 26). The boxes represent residues of alpha-helices (H3, H4, H5, H6 and H12); lower case letters “h” and “q” represent hydrophobic and polar residues, respectively. The numbered positions, Leu712, Val716, etc., above the sequence alignments identify AR coactivator binding site residues.

FIG. 5, comprising FIGS. 5A-5F, was generated and rendered using PyMOL (DeLano, (2002)) from coordinates of complexes, as shown in Table 2 in the file identified as Table2_ARLBD_DHT_CRP.txt, presented on CD-R herewith, and shows a comparison of the binding of 5 coactivator-related peptides to the AR coactivator binding site. In FIG. 5A, the structures of the coactivator related peptides CRP_{—}1, CRP_{—}3, and CRP_{—}4 were overlapped using their respective Ca coordinates. The core hydrophobic motif of each peptide forms a short helix which binds in a groove formed by helices 3, 4, 5, and 12 of the coactivator binding site, which are labeled H3, H4, H5, and H12, respectively. FIGS. 5B, 5C, 5D, 5E, 5F, 5G and 5H show, respectively, the binding of each of peptides CRP_{—}1, CRP_{—}3, CRP_{—}4, CRP_{—}5, CRP_{—}2, CRP_{—}6, and CRP_{—}7 into the AR coactivator binding site. The following abbreviations are used on the coactivator binding site: Q733=Gln 733, K720=Lys 720, and M734=Met 734, Q738=Gln 738, M894=Met 894, and E897=Glu 897. For each coactivator peptide, side chains on residues in the +1, +4, and +5 positions are shown.

FIGS. 6A to 6E were generated using and rendered using the program PyMOL (DeLano, (2002)) from coordinates of complexes, as shown in the files identified as Table2_ARLBD_DHT_CRP.txt, presented on CD-R herewith. Each figure shows the interaction between a coactivator-related peptide and the coactivator binding site of AR, as determined by the methods of the present invention. FIG. 6A shows CRP_{—}1; FIG. 6B shows CRP_{—}2; FIG. 6C shows CRP_{—}3; FIG. 6D shows CRP_{—}4; FIG. 6E shows CRP_{—}5; FIG. 6F shows CRP_{—}6; and FIG. 6G shows CRP_{—}7. In each figure, receptor residues Gln733, Phe725, Lys720, Val730, Ile737, Val 716, Val 713, Met 734, Gln738, Leu712, Met894, Glu893, Glu897, and Ile898 that define the AR coactivator binding site are identified. H6 residue Trp 741 is hidden, and therefore not labelled. The coactivator residues at positions +1, +4, and +5 of the hydrophobic motif are also identified in each figure, and the N- and C-termini of the coactivator peptide are indicated.

FIG. 7 provides an illustration of a computer system for use in the present invention.

FIG. 8 shows SDS-Page data for purification of androgen receptor protein. The first gel shows the initial purification steps over the Glutathione-4 Fast Flow resin. The arrow denotes GST-AR LBD fusion protein. The second gel shows the progression of the thrombin cleavage reaction to separate the GST and AR LBD moieties. The third gel shows the final purified AR LBD product eluted from the Mono-S resin.

FIG. 9, comprising FIGS. 9A-9E, provides binding data curves for 3 coactivator peptides, CRP_{—}1, CRP_{—}3, CRP_{—}4, and a coregulatory peptide, SMRT2B, according to the present invention. FIGS. 9A, 9B, 9C, and 9E display overlay plots of 4 different concentrations of AR LBD protein (10, 5, 1 and 0.3 μM) interacting with peptides CRP_{—}1, CRP_{—}3, CRP_{—}4, and SMRT2B, respectively. FIG. 9D displays the relative unit response of each of the four biotinylated peptides as they bind irreversibly to distinct streptavidin coated flow cell channels.

FIG. 10, comprising FIGS. 10A and 10B, shows the structures of two molecules, of formulae (I) and (II) respectively, designed by the methods of the present invention, fit into a model of the androgen receptor coactivator binding site obtained by the methods of the present invention. Residues that form the coactivator binding site are indicated. Val 730 is in close contact with the indole ring of the two molecules (I) and (II). Residue Gln 738 is shown but is not in close contact with the molecules (I) and (II).

FIG. 11 is a schematic diagram illustrating the interactions of coactivator-related peptide CRP_{—}1 having sequence SSRGLLWDLLTKDSR (SEQ ID NO: 6) with the AR coactivator binding site. FIG. 11 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (A), presented on CD-R herewith. The general features of the drawing are as described for FIG. 5, herein, except that crystallographic water molecules are labeled “HOH” and the numbering of residues is according to the structure presented in the file identified as Table2_ARLBD_DHT_CRP.txt at (A), presented on CD-R herewith.

FIG. 12 is a schematic diagram illustrating the interactions of coactivator-related peptide CRP_{—}2 with sequence SRWQALFDDGTDTSR (SEQ ID NO: 7) WITH THE ar coactivator binding site. FIG. 12 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (B), presented on CD-R herewith. The general features of the drawing are as described for FIG. 5, herein, except that the numbering of residues is according to the structure presented in the file identified as Table2_ARLBD_DHT_CRP.txt at (B), presented on CD-R herewith.

FIG. 13 is a schematic diagram illustrating the interactions of coactivator-related peptide CRP_{—}3 with sequence SSRFESLFAGEKESR (SEQ ID NO: 8) with the AR coactivator binding site. FIG. 13 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (C), presented on CD-R herewith. The general features of the drawing are as described for FIG. 5, herein, except that crystallographic water molecules are labeled “HOH” and the numbering of residues is according to the structure presented in the file identified as Table2_ARLBD_DHT_CRP.txt at (C), presented on CD-R herewith.

FIG. 14 is a schematic diagram illustrating the interactions of coactivator-related peptide CRP_{—}4 with sequence SSKFAALWDPPKLSR (SEQ ID NO: 9) with the AR coactivator binding site. FIG. 14 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (D), presented on CD-R herewith. The general features of the drawing are as described for FIG. 5, herein, except that crystallographic water molecules are labeled “HOH” and the numbering of residues is according to the structure presented in the file identified as Table2_ARLBD_DHT_CRP.txt at (D), presented on CD-R herewith.

FIG. 15 is a schematic diagram illustrating the interactions of coactivator-related peptide CRP_{—}5 with sequence SRFADFFRNEGLSGSR (SEQ ID NO: 10) with the AR coactivator binding site. FIG. 15 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (E), presented on CD-R herewith. The general features of the drawing are as described for FIG. 5, herein, except that crystallographic water molecules are labeled “HOH” and the numbering of residues is according to the structure presented in the file identified as Table2_ARLBD_DHT_CRP.txt at (E), presented on CD-R herewith.

FIG. 16 is a schematic diagram illustrating the interactions of coactivator-related peptide CRP_{—}6 with sequence SSNTPRFKEYFMQSR (SEQ ID NO: 11) with the AR coactivator binding site. FIG. 16 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (F), presented on CD-R herewith. The general features of the drawing are as described for FIG. 5, herein, except that crystallographic water molecules are labeled “HOH” and the numbering of residues is according to the structure presented in the file identified as Table2_ARLBD_DHT_CRP.txt at (F), presented on CD-R herewith.

FIG. 17 is a schematic diagram illustrating the interactions of coactivator-related peptide CRP_{—}7 with sequence SRWAEVWDDNSKVSR (SEQ ID NO: 12) with the AR coactivator binding site. FIG. 16 was generated using LIGPLOT (Wallace, et al., Protein Eng., 8:127-34, (1995)) from atomic structural coordinates obtained from a crystal of the present invention, and presented in the file identified as Table2_ARLBD_DHT_CRP.txt at (G), presented on CD-R herewith. The general features of the drawing are as described for FIG. 5, herein, except that crystallographic water molecules are labeled “HOH” and the numbering of residues is according to the structure presented in the file identified as Table2_ARLBD_DHT_CRP.txt at (G), presented on CD-R herewith.

FIG. 18, comprising FIGS. 18A and 18B, shows for purposes of comparison, ribbon diagrams of the AR LBD, including the coactivator binding site, when bound with the ligand DHT, and a coactivator-derived peptide. FIGS. 18A and 18B were obtained using atomic structural coordinates obtained from crystals of the present invention, and found in the file identified as Table1_ARLBD_DHT_CDP.txt, presented on CD-R herewith. The coactivator-derived peptide in FIG. 18A contains part of the ARA70 binding motif; that in FIG. 18B contains the GRIP1 NR-box3 binding motif. In each case, the coactivator peptide is shown as a light-colored helix. Helices comprising the AR LBD are shown labeled H1, and H3 through H12; H2 is not shown because, when a hormone such as DHT is bound, it is virtually non-existent. When a hormone is bound to the LBD, the hydrophobic face of helix 12 is packed against helices 3, ⅚ and 11 covering the ligand binding pocket. The N-terminus of the AR LBD is also indicated. The positions of the side chains of AR LBD residues K720 and E897 are shown, thus indicating the different conformations adopted between the receptors with the two coactivator peptides. FIGS. 18A and 18B were generated with PyMOL, (see, DeLano, W. L., “The PyMOL Molecular Graphics System”, (2002), available from DeLano Scientific, San Carlos, Calif., USA, see also www.pymol.org).

FIG. 19, comprising FIGS. 19A and 19B show a close-up view of the coactivator-derived peptide with sequence RETSEKFKLLFQSYN (SEQ ID NO: 13), derived from ARA70, bound to the coactivator binding site of AR when DHT (not shown) is also in the LBD. FIGS. 19A and 19B were obtained using atomic structural coordinates obtained from crystals of the present invention, and presented in the file identified as Table1_ARLBD_DHT_CDP.txt, presented on CD-R herewith. The regions of the coactivator binding site that do not interact with the peptide have been omitted for clarity. In FIG. 19A, helices 3, 4 and 12 are labeled H3, H4 and H12 respectively. The side chains of receptor residues K720, M734, and E897, which interact with the peptide, are also depicted in FIG. 19A. Side chains of other receptor residues in the coactivator binding site are shown. In both FIGS. 19A and 19B, the coactivator peptide is depicted as a Cα worm; only the side chains of the three motif residues (Phe+1, Leu+4, and Phe+5) are shown. The view depicted in FIG. 19A is equivalent to that in FIG. 19B, except that the receptor surface is shown shaded in FIG. 19B. The N- and C-termini of the coactivator peptide are also indicated in FIG. 19B. The side chains of Phe+5 and Phe+1 of the coactivator peptide are bound in a hydrophobic groove. FIGS. 19A and 19B were generated with PyMOL (DeLano, (2002)).

FIGS. 20A and 20B were generated using PyMOL (DeLano, (2002)), and show a close-up view of the coactivator-derived peptide with sequence KENALLRYLLDKDD (SEQ ID NO: 14), derived from GRIP1 Box 3, bound to the coactivator binding site of AR when DHT (not shown) is also in the LBD. FIGS. 20A and 20B were obtained using atomic structural coordinates obtained from crystals of the present invention, and presented in the file identified as Table1_ARLBD_DHT_CDP.txt, presented on CD-R herewith. The regions of the coactivator binding site that do not interact with the peptide have been omitted for clarity. In FIG. 20A, helices 3, 4 and 12 are labeled H3, H4 and H12 respectively. The side chains of receptor residues K720, M734, and E897, which interact with the peptide, are also depicted in FIG. 20A. [Side chains of other receptor residues in the coactivator binding site are shown. In both FIGS. 3A and 3B, the coactivator peptide is depicted as a Cα worm; only the side chains of the three motif residues (Leu+1, Leu+4, and Leu+5) are shown. The view depicted in FIG. 20A is equivalent to that in FIG. 3B, except that the receptor surface is shown shaded in FIG. 20B. The N- and C-termini of the coactivator peptide are also indicated in FIG. 19B. The side chains of Leu+5 and Leu+1 of the coactivator peptide are bound in a hydrophobic groove.

FIG. 21, comprising FIGS. 21A and 21B both of which were generated with PyMOL (DeLano, (2002)), shows a comparison of the coactivator binding pocket with a GRIP1-Box3-derived peptide with sequence KENALLRYLLDKDD (FIG. 21A, wherein the peptide is a light-colored Cα worm) and an ARA70-derived peptide (FIG. 21B, wherein the peptide is a dark-colored Cα worm). FIGS. 21A and 21B were obtained using atomic structural coordinates obtained from crystals of the present invention, and presented in the file identified as Table1_ARLBD_DHT_CDP.txt, presented on CD-R herewith. The binding pocket that accommodates the ARA70-derived peptide is larger than that of the pocket that accommodates the GRIP1 NR-box3-derived peptide.

FIG. 22 was generated using LIGPLOT (Wallace, et al., Protein Eng., 8:127-34, (1995)) and provides a schematic diagram illustrating the interactions of GRIP1-Box3-derived peptide with sequence KENALLRYLLDKDD with the AR coactivator binding site. FIG. 22 was obtained using atomic structural coordinates obtained from a crystal of the present invention, and presented in the file identified as Table1_ARLBD_DHT_CDP.txt, presented on CD-R herewith. Residues that interact with the coactivator peptide are drawn at approximately their true positions. The residues that form van der Waals contacts with coactivator peptide are depicted as labeled arcs with radial spokes that face towards the ligand atoms with which they interact. The residues that hydrogen bond to ligand are shown in ball-and-stick representation. Hydrogen bonds are represented as dashed lines and the distance of each bond (in Å) is given. The individual coactivator peptide atoms are labeled; crystallographic water molecules are labeled “Tip”. Numbering of residues is according to the structure presented in the file identified as Table1_ARLBD_DHT_CDP.txt, presented on CD-R herewith.

FIG. 23 was generated using LIGPLOT (Wallace, et al., Protein Eng., 8:127-34, (1995)) and provides a schematic diagram illustrating the interactions of ARA70-derived peptide with sequence RETSEKFKLLFQSYN with the AR coactivator binding site. FIG. 23 was obtained using atomic structural coordinates obtained from crystals of the present invention, and presented in the file identified as Table1_ARLBD_DHT_CDP.txt, presented on CD-R herewith. General features of the drawing are as described for FIG. 23, herein, except that numbering of residues is according to the structure presented in the file identified as CDP_ARA70.txt, presented on CD-R herewith.

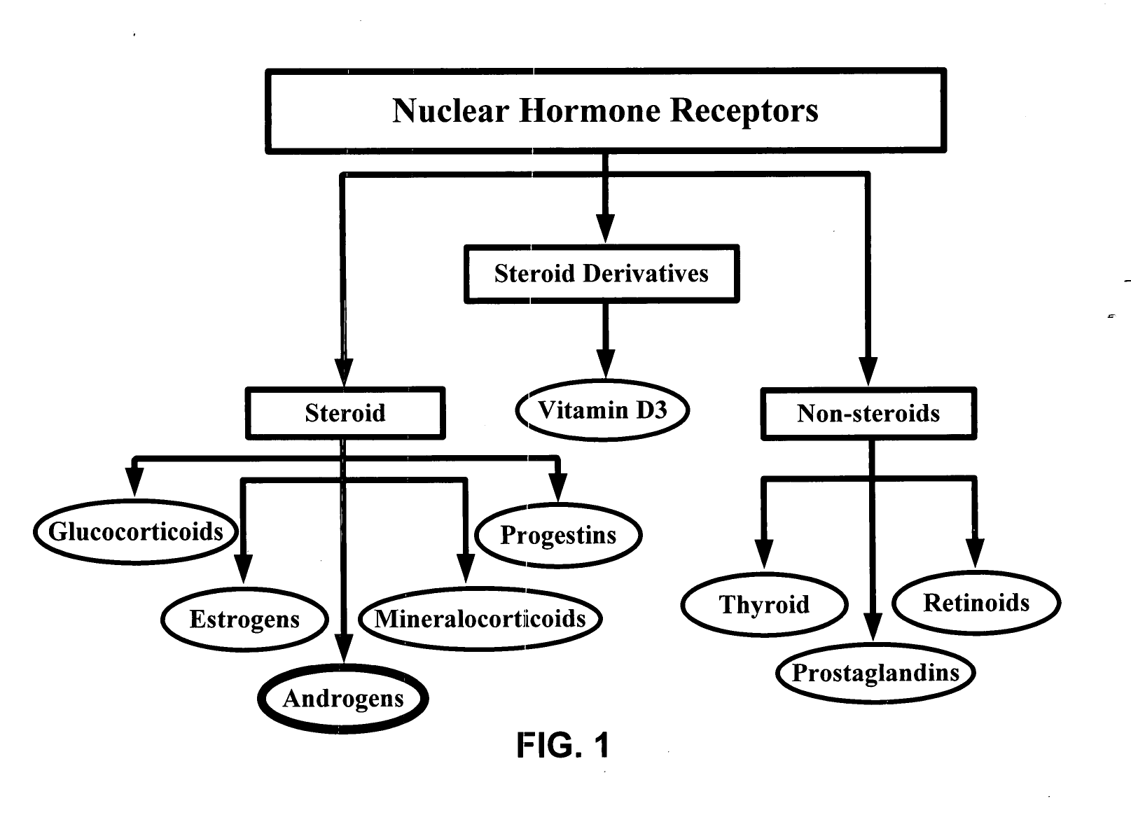

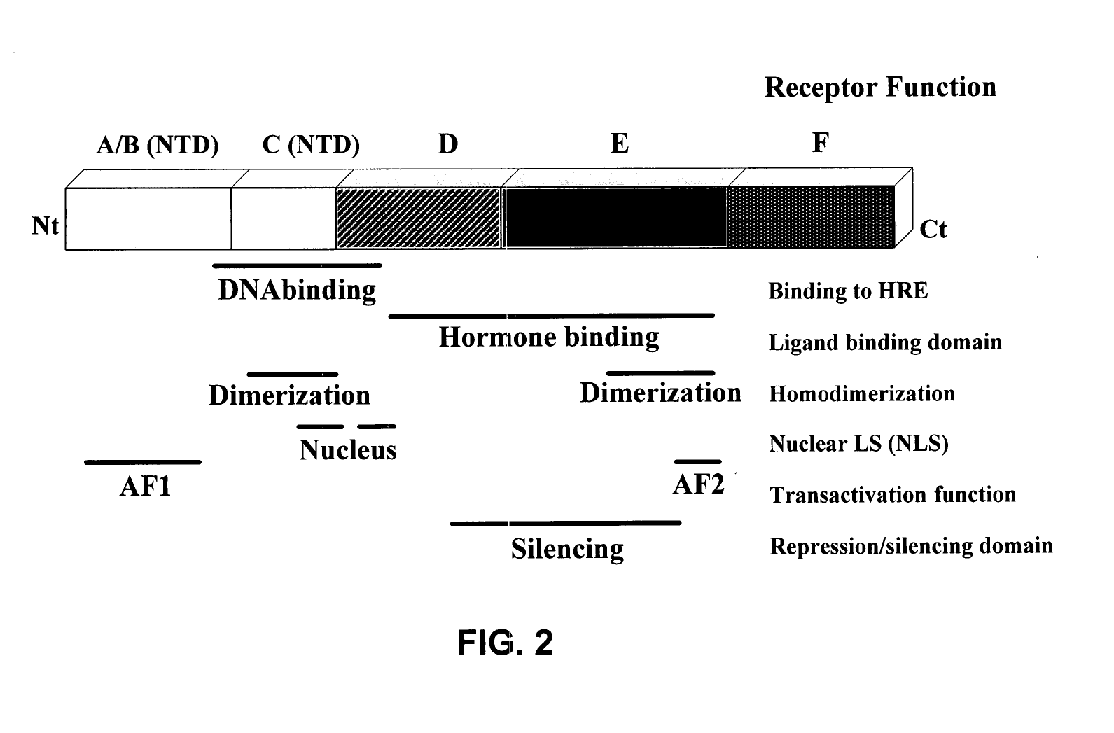

### DETAILED DESCRIPTION OF THE INVENTION

Despite the importance of nuclear receptors in a myriad of physiological processes and medical conditions such as hypertension, inflammation, hormone dependent cancers (e.g., breast and prostate cancer), modulation of reproductive organ modulation, hyperthyroidism, hypercholesterolemia and obesity, identification of compounds that modulate nuclear receptor activity has been hampered by a lack of structural information.

Accordingly, the present invention provides atomic structural information about the coactivator binding site of nuclear receptors, in particular steroid receptors, and more particularly the androgen receptor. Specifically, the present invention provides crystallographic data for portions of the androgen receptor ligand binding domain, to which is bound an agonist molecule, and to which is additionally bound a coactivator molecule, situated in the coactivator binding site. From the crystallographic data of the present invention, the determinants of interaction between a coactivator and a nuclear receptor such as the androgen receptor are identified.

The present invention also provides methods for identifying compounds that modulate nuclear receptor activity, in particular steroid receptor activity, and more particularly androgen receptor activity. The present invention further provides compounds and compositions thereof that are suitable for modulating muclear receptor activity, in particular steroid receptor activity, and more particularly androgen receptor activity. By “modulate”, or “modulating” is intended increasing or decreasing activity of the nuclear receptor. The compounds are nuclear receptor antagonists that bind to the coactivator binding site. The compounds can be natural or synthetic. Preferred compounds are small organic molecules, peptides and peptidomimetics (e.g., cyclic peptides, peptide analogs, or constrained peptides). Preferably the compounds inhibit the binding of other, endogenous, coactivators to the coactivator binding site, thereby inhibiting receptor function.

The terms “coactivator” and “coregulator” are often used interchangeably herein and in the art. It is to be understood that a “coactivator” means a molecule, or part thereof, that binds to the coactivator binding site of a nuclear receptor, in particular the androgen receptor. Thus the term “coactivator” comprises molecules that are both naturally occurring, endogenous, and those designed and/or synthesized in the laboratory. In particular, the term coactivator encompasses peptide molecules that comprise sequences that are found in naturally occurring coactivator molecules and which play a role in coactivator binding. Furthermore, such coactivator molecules preferably have modulatory effect on nuclear receptor function: that is, they may enhance or repress nuclear receptor activity, such as transactivation.

The term “coregulator” may also find use herein; as such, a coregulator typically refers to a naturally occurring molecule that, from its binding to the coactivator binding site, has the effect either to enhance or to repress nuclear receptor function. The term “corepressor” may also find use herein, to mean a molecule that represses nuclear receptor activity through binding to the nuclear receptor coactivator binding site. Consequently, the terms coactivator, and coregulator, as used herein, both encompass molecules that would otherwise be referred to as corepressors.

Coactivator peptides for use in conjunction with the methods of the present invention are preferably obtained according to one of two ways. “Coactivator-related” peptides are synthetic peptides that contain a motif, such as LXXLL or FXXLF, that is suitable for binding to the coactivator binding site, but have been selected from a screening protocol such as one described herein. Such peptides are referred to as coactivator-related because they contain a motif that is present in known coactivators, but, outside of the motif in question, their sequences comprise an essentially random selection of residues that is not found in naturally occurring coactivators. By contrast, “coactivator-derived” peptides are peptides that comprise sequences that are found in known physiological coactivators such as GRIP, or ARA70, and also include a motif such as LXXLL, or FXXLF that is also found in a known physiological coactivator. Thus, residues flanking the motif are also found in the known coactivator.

The terms “molecule” and “compound” find use herein. It is to be understood that the term “molecule” can mean a single molecule of a substance or can refer to the substance itself in the sense that a molecule has a unique identity and confers unique properties upon the substance. The term “compound” typically refers to an aggregate of molecules of a substance, as may be physically handled in a laboratory. However, the term compound may also be used to refer to a single molecule where a manipulation, for example, a computer simulation, is taking place in which the atomic structure of the molecule is understood and utilized.

Molecules which preferentially bind each other are typically referred to herein by the terms “receptor” and “ligand”. Usually, the term “receptor” is assigned to a member of a specific binding pair which is of a class of molecules known for its binding activity, e.g., a protein. The term “receptor” is also preferentially conferred on the member of the pair which is larger in size, e.g., on a nuclear receptor in the case of a nuclear-receptor-hormone pair. However, the identification of receptor and ligand is occasionally arbitrary, and the term “ligand” may be used to refer to a molecule such as a protein which has a separate “receptor” function. In general, hen, the term “ligand”, as used herein, refers to a molecule that binds to a receptor; the molecule may be a peptide, peptidomimetic, small organic molecule, or protein.

When two molecules, such as a ligand and a receptor, bind one another, they are considered to interact in such a way that contacts are formed between atoms on one molecule and those on the other. Such contacts preferably include non-covalent interactions variously described as hydrogen bonding interactions, van der Waals interactions, electrostatic interactions such as charge, dipolar, and quadrupolar interactions, or a hydrophobic interaction. It is understood that such interactions are individually weaker than covalent interactions, but when aggregated over a number of different pairs of atoms, may amount to a considerable energetic quantity. Such interactions are typically called long-range interactions, because they occur over distances that are longer than the length of a covalent bond, and are often influenced considerably by the presence of molecules of a solvent such as water. It is also to be understood that interactions between two molecules, according to the present invention, may further comprise one or more covalent interactions such as the formation of a covalent bond, or a dative covalent bond between the two molecules.

By “fits spatially and preferentially” is intended that a compound possesses a three-dimensional structure and conformation that is accommodated geometrically by a cavity or pocket on the surface of a protein. Such a compound possesses requisite features for selectively interacting with a binding site such as a coactivator binding site of a nuclear receptor LBD. Compounds of the present invention that fit spatially and preferentially into the LBD interact with amino acid residues forming the hydrophobic cleft of this site.

According to the present invention, using methods described herein, isolated and purified samples of a complex comprising an androgen receptor ligand binding domain have been obtained in which a ligand such as a known hormone is bound to the ligand binding domain, and in which a coactivator is bound to a coactivator binding site. Such samples have been crystallized, also using procedures described herein, so that atomic structural coordinates of portions of the ligand binding domain, ligand bound thereto, and coactivator molecule, have been obtained.

The present invention preferably comprises an isolated and purified sample, and a cocrystal thereof, of a complex comprising an androgen receptor ligand binding domain in which a ligand such as a known hormone is bound to the ligand binding domain, and in which a coactivator is bound to a coactivator binding site, wherein the coactivator is a peptide whose sequence comprises a motif that is found in a coactivator that binds to coactivator binding sites across the nuclear receptor family including AR, or comprises a motif that is found in a coactivator that preferentially binds to the AR coactivator binding site, or comprises a motif that is found in the N-terminal domain of AR and which binds to the AR coactivator binding site.

The term “atomic structural information”, as used herein, is taken to mean coordinates and identities of atoms found in a molecule or complex, presented or stored in any one of the formats referred to hereinbelow. From atomic structural information it is typically possible to deduce further information important to a chemist, such as the location and type of chemical bonds between atoms in the molecule or complex. It is further to be understood that atomic structural information may be incomplete in the sense that one or more atoms, particularly hydrogen atoms, is missing. However, where there are such missing atoms, it is further to be understood that one of ordinary skill in the art is usually able to deduce the likely position and identity of such atoms, particularly using one or more software programs that would be readily available. The term “atomic model”, or “atomic structural model” may also find use herein. Such terms refer to a set of identities and coordinates for the atoms in a molecule presented in such a way that a 3-dimensional representation of the molecule may be presented to one of ordinary skill in the art on, for example, a computer display. Such a 3-dimensional representation may be further manipulated by, for example, rotating or translating it on the display, or by altering its conformation so that the 3-dimensional disposition of its constituent atoms is changed, even though the way in which they are bonded to one another remains unchanged.

The amino acid notations used herein for the twenty genetically encoded L-amino acids are the conventional one-letter (A, C, D, etc.) and three-letter (Ala, Arg, Cys, etc.) abbreviations familiar to one of ordinary skill in the art. As used herein, unless specifically delineated otherwise, the three-letter amino acid abbreviations designate amino acids in the L-configuration. Likewise, the capital one-letter abbreviations refer to amino acids in the L-configuration. Furthermore, unless noted otherwise, when polypeptide sequences are presented as a series of one-letter and/or three-letter abbreviations, the sequences are presented in the N→C direction, in accordance with common practice wherein “N” refers to the amino terminus of a polypeptide, and “C” refers to the carboxy terminus of a polypeptide.

Description of the NR Coactivator Binding Site

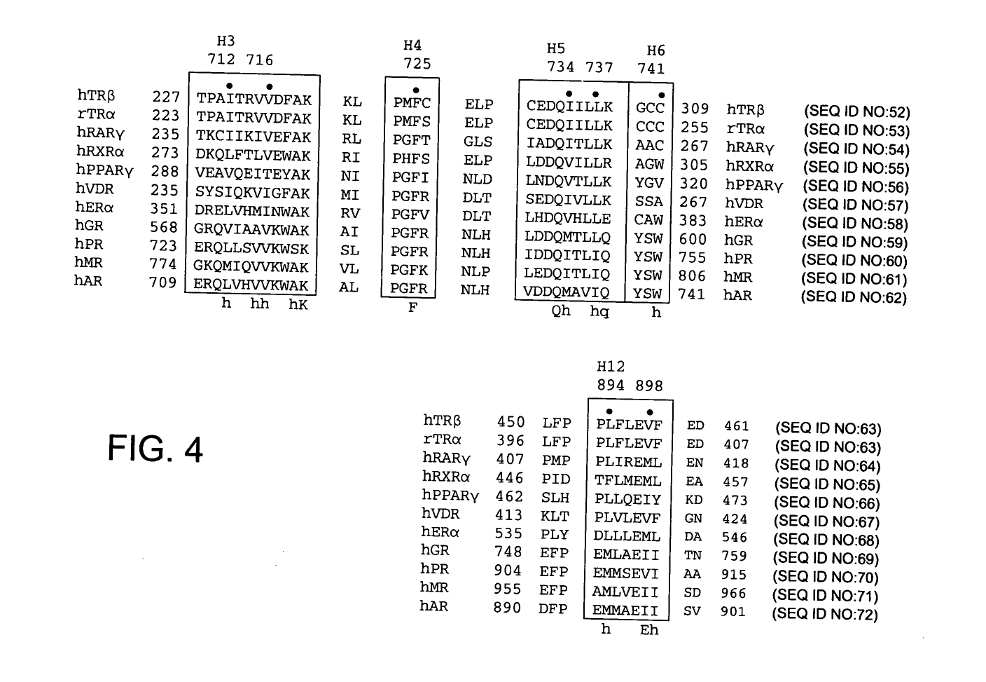

The term “coactivator binding site” is used herein to mean a structural segment, or segments, of the nuclear receptor polypeptide chain folded in such a way as to give the proper geometry and amino acid residue conformation for binding a coactivator. This is the physical arrangement of protein atoms in three-dimensional space that form a coactivator binding site pocket or cavity. As described by Apriletti, et al., (U.S. Pat. App. Pub. No. 2002/0061539 A1, May 23, 2002) the coactivator binding site corresponds to a surprisingly small cluster of residues on the surface of the LBD that form a prominent hydrophobic cleft. Residues that form the coactivator binding sites of a number of nuclear receptors are shown in FIG. 4. Certain residues that form the coactivator binding site are highly conserved among the nuclear receptor super family.

The preparation and analysis of cocrystals of nuclear receptors with agonist/antagonist ligands and coactivators bound respectively to the LBD and the coactivator binding site, according to the present invention, has allowed many structural aspects of the NR coactivator binding site to be deduced. As described hereinabove, many coactivators recognize agonist bound nuclear receptor LBD's through the sequence motif LXXLL (SEQ ID NO: 1), where L is leucine and X is any amino acid, a motif which is also referred to as the nuclear receptor box (“NR-box”). The LXXLL motif (SEQ ID NO: 1) forms the core of a short amphipathic α-helix which is recognized by a highly complementary hydrophobic groove on the surface of the nuclear receptor. This peptide binding groove is the coactivator binding site and is formed by residues from helices 3, 4, 5 and 12 and the turn between helices 3 and 4. The groove lies on the surface of a nuclear receptor ligand binding domain. The floor and sides of this groove are completely nonpolar, but the ends of this groove are charged. These features have also been seen in the structure of the DES-hERα LBD-GRIP1 peptide complex. Furthermore, structural studies of the complex between TRβ and the GRIP1 NR box 2 peptide, biochemical studies of GRIP1 binding to TRβ and GR (Darimont, et al., Genes Dev., 12:3343-3356, (1998)), and a study of the general features of the PPARγ/SRC-1 peptide complex (Nolte, et al., Nature, 395:137-143, (1998)) suggest that certain mechanisms of NR box recognition are probably conserved across the nuclear receptor family. Nevertheless, differences between the coactivator binding sites, and ligand binding domains, of various nuclear receptors have emerged, and a definitive understanding of the structure of a given coactivator binding site is facilitated by having access to a crystal structure thereof, particularly one comprising a bound coactivator.

It is thus to be understood that, although the coactivator binding site lies within the ligand binding domain of a given nuclear receptor, it is a specific feature thereof, and that a knowledge of the architecture of a given LBD itself is not always sufficient to identify distinguishing characteristics of the coactivator binding site within. In particular, although residues that form the coactivator binding site of a particular nuclear receptor such as AR may be known, or may be inferred, say, by homology with other nuclear receptors, the distinguishing structural and electrostatic characteristics of the coactivator binding site may not also be known, and its characterization is thus facilitated by examining the structure of the LBD and coactivator binding site with a coactivator molecule bound thereto.

Furthermore, it also to be understood that a ligand binds to the LBD of a nuclear receptor in a manner that is separate and independent of coactivator binding to the coactivator binding site. That is, the location and orientation of a ligand bound to the LBD are different from the respective location and orientation of a coactivator bound to the coactivator binding site, even though the coactivator binding site is considered to be a specific feature on the surface of the LBD.

The NR box motifs themselves have certain structural characteristics that facilitate binding to the coactivator binding site. The hydrophobic face of the NR box helix LXXLL is formed by the side chains of the three motif leucines and, in the case of GRIP1 Box 2, the isoleucine that precedes the motif. The functional importance of the conserved NR-box leucines in receptor binding has been demonstrated by numerous in vitro and in vivo studies. Accordingly, peptides and peptidomimetics of the present invention that are designed to bind to the coactivator binding site preferably have a hydrophobic face that mimics the hydrophobic face, and in particular the leucines, of an NR box motif. The charged and polar side chains which form the hydrophilic face of the peptide helix project away from the receptor and interact predominantly with solvent.

It is also important to recognize that certain sequences of residues outside of, and adjacent to, the NR-box motifs may play a role in coactivator recognition and binding, and thus mimicing their properties may be important. Such residues are referred to herein as “flanking” residues. In contrast to the NR-box leucines themselves, the Ile residue that flanks the GRIP1 Box2 is less well conserved amongst coactivators. Both biochemical and structural data implicate this isoleucine as a key receptor binding determinant. Mutation of the isoleucine to alanine has been shown to reduce the ability of the coactivator peptide to inhibit the binding of GRIP1 to ERα by 30 fold in a competition assay. In known crystal structures, only the side chains of the motif leucines and the flanking Ile residue extensively contact the coactivator binding site. The side chain of this Ile residue lies in a rather chemically distinct environment in the coactivator binding site. For example, in ERα the Ile residue forms van der Waals contacts with the aliphatic portion of the ER Asp 538 side chain, the side chain of ER Leu 539 and the γ-carboxylate of ER Glu 542. It is thought that the proximity of this negatively charged moiety of ER Glu 542 to the hydrophobic side chain of the flanking isoleucine in the coactivator enhances the electrostatic potential of the side chain carboxylate and strengthens its stabilizing interactions with the N-terminus of the coactivator helix. Accordingly, peptide coactivators of the present invention preferably have an isoleucine residue in a flanking position corresponding to the flanking Ile in GRIP1 NR Box2.

Despite its apparently important role in receptor recognition, the identity of the residue immediately preceding known NR boxes is poorly conserved. This sequence variability has effects not only on packing interactions with a nuclear receptor but also on both the chemical environment and the orientation of an important residue such as that corresponding to Glu 542 in ERα. This in turn translates into variations in affinity for the receptor amongst different NR-box motifs. Indeed, the three NR boxes from GRIP1, which each contain a different residue preceding the LXXLL motif (SEQ ID NO: 2), have differing affinities for the nuclear receptor, ERα (Ding. et al. Mol. Endocrinol, 12:302-313, (1998). Voegel, et al. EMBO J. 17:507-19, (1998)). Accordingly, it is consistent with the coactivators of the present invention, that certain flanking residues may be chosen to ensure receptor specificity of interactions. In particular, it is preferable to choose flanking residues that ensure binding to the androgen receptor.

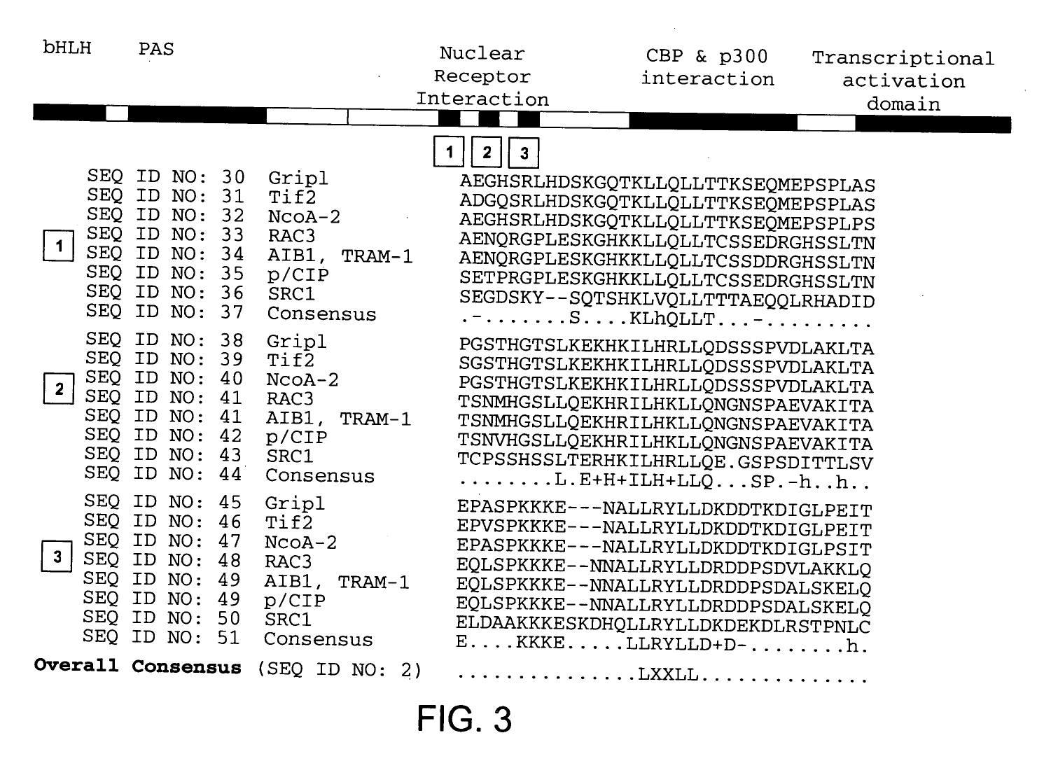

Data has indicated that a single NR box motif is sufficient to form a tightly bound complex with a single nuclear receptor LBD, such as that of ERα. Yet some coactivators, for example those in the p160 family, possess multiple NR boxes. Multiple NR-boxes in various coactivator peptides are shown in FIG. 3, and are labeled “Box 1”, “Box 2”, and “Box 3”. A possible explanation for the presence of multiple NR boxes is that they provide coactivators with broad specificity. The various nuclear receptors have some differences in their coactivator binding site surfaces. Since receptor binding relies upon the intricate formation of multiple van der Waals interactions, the different amino acids in the position immediately preceding the LXXLL motif (SEQ ID NO: 2) might allow some degree of adaptability to these distinct surfaces. Multiple NR boxes may therefore provide coactivators with the diversity of interfaces necessary to recognize a variety of targets. Accordingly, it is a further property of peptide coactivators of the present invention that they may contain more than one sequence motif that corresponds to a NR-box peptide motif.

Coactivator peptides used with the present invention preferably comprise sequences that include an NR box and flanking residues that are the same as or are homologous to residues found adjacent to NR box motifs in the p160 coactivator family. In particular, coactivator peptides of the present invention preferably comprise at least one sequence selected from the group consisting of: NR Box 1, NR Box 2, and NR Box 3 sequences. Still more preferably, the peptides of the present invention comprise an NR Box 3 sequence that includes and NR Box motif and flanking residues.

Examination of the structures of the complexes of the present invention reveals that some features of the AR coactivator binding site are common to those of other known nuclear receptors. In particular, certain conserved residues are found in the coactivator binding site of AR, and certain hydrophobic residues on the surface of the AR coactivator binding site correspond to hydrophobic residues in other nuclear receptors. The hydrophobic cleft that defines the coactivator binding site of AR is formed by hydrophobic residues including, but not limited to, N-terminal helix 3 (Leu 712, Val713, Val716, Lys 720), helix 4 (Phe725), helix 5 (Val 730, Gln 733, Met734, Ile737, and Gln738), helix 6 (Trp741), and C-terminal helix 12 (Glu 893, Met894, Glu897 and Ile898). The predominant interactions are with residues in helices 3, 4, 5, and 12. The Trp741 residue of helix 6 is at the bottom of the coactivator binding site, and is largely buried by the +1 pocket residues Ile 898 and Leu 712. Although, such residues forming the AR coactivator binding site are homologous to residues that define the coactivator binding sites of other nuclear receptor family members, there are differences between the AR coactivator binding site, and the coactivator binding sites of other nuclear receptors, as further described herein.

The formation of helix capping interactions is probably a general feature of coactivator recognition by nuclear receptors. The side chains of AR residues Lys 720 and Glu 897 are largely solvent exposed in the absence of coactivator, but when a coactivator is bound, these residues make both nonpolar contacts and key receptor-mediated polar interactions with the coactivator helix. These two capping interaction residues are well positioned at opposite ends of the coactivator binding site groove, not only to stabilize the main chain conformation of the coactivator, but also to function as a molecular caliper; the distance between Lys 720 and Glu 897 is well suited to accommodate the axial length of the short, two-turn coactivator α helix. However, while Lys 720 makes an interaction with most coactivator peptides studied herein, Glu 897 only interacts with some of them. Similar receptor-mediated capping interactions have also been observed in a complex between the TRβ LBD and the NR box II peptide (Darimont, et al., Genes Dev., 12:3343-3356, (1998)). Mutation of either of the two residues corresponding to AR residues Lys 720 and Glu 897 severely cripples coactivator binding in other nuclear receptors such as ERα and TRβ (see Apriletti, et al., U.S. Pat. App. Pub. No. 2002/0061539 A1, Feng, et al., Science, 280:1747-9, (1998); Henttu, et al., Mol. Cell. Biol., 17:1832-9, (1997)).

The side chains of conserved hydrophobic coactivator binding residues such as Leu 712, Val 716, Val 730, Met 734, Ile 737, Met 894 and Ile 898 form part of a highly cooperative network of van der Waals contacts made by the receptor with the hydrophobic face of the coactivator helix. Although these residues are, in general, more poorly conserved across nuclear receptors than either Lys 720 or Glu 897, (see FIG. 3) their hydrophobic character, with the exception of Val 730, is conserved. Mutations in ERα residues Ile 358, Val 376 and Leu 539, corresponding respectively to AR residues Val 716, Met 734, and Met 894, abrogate GRIP1 binding (see Feng, et al., Science, 280:1747-9, (1998)).

Since many different NR LBD's adopt a similar overall fold (Moras, et al., Curr. Opin. Cell Biol., 10:384-91, (1998)), in order to account for receptor specificity it follows that the hydrophobic regions of different nuclear receptor coactivator binding site surfaces are distinctly textured from one another. For example, the NR box 2 peptide used in crystallization of AR LBD, as described herein, inhibited binding of GRIP1 to the LBD's of the ERα, the TRβ and the glucocorticoid receptor (GR) with very different efficiencies (Ding, et al., Mol. Endocrinol, 12:302-313, (1998)).

Thus, the manner of inhibiting coactivator binding to AR according to methods of the present invention differs from that of another member of the nuclear receptor family, ERα. In ERα, antagonist binding to the LBD (which blocks transcriptional activity) causes a structural reorganization in which helix 12 becomes bound to the static region of the coactivator recognition groove (see, e.g., international publication no. WO99/50658). This is because in ERα there is a region of helix 12 that has an NR box-like sequence (LXXML (SEQ ID NO: 15)) that functions as an intramolecular mimic of the LXXLL motif of the helix in a coactivator such as p160. This disposition of helix 12 in ERα directly affects the structure and function of the surface responsible for transcriptional activity in two ways. First, because helix 12 residues form an integral part of the coactivator binding site surface, the surface is incomplete when helix 12 is in the antagonist-bound conformation. Second, residues from the static region of the coactivator binding surface are bound to helix 12 and are prevented from interacting with coactivator. Thus, when an antagonist such as OHT binds to ERα, it does not directly interact with any helix 12 residues. The identities of the residues in this region of helix 12 in other nuclear receptors, although generally hydrophobic in character, do not as closely resemble the sequence of an NR box as those of ERα (Wurtz, et al., Nat. Struct. Biol., 3:87-94, (1996)).

The AR Coactivator Binding Site and Features of AR Coactivator Binding According to the Present Invention

The AR is a member of the steroid receptor group whose members are encoded by the NR3C gene group. Specifically, AR is encoded by the NR3C4 gene. The accession number of the nucleotide sequence of the gene that encodes the human AR is M20132. The AR has about 900-920 amino acid residues, and has been discovered in a large number of vertebrates, including, but not limited to: human, chimpanzee, baboon, macaque, lemur, mouse, rat, rabbit, sheep, dog, canary, green anole, xenopus, rainbow trout (α and β forms), Japanese eel (α and β forms), and red seabream (see, e.g., Gronemeyer, H., and Laudet, V., The Nuclear Receptor Facts Book, p. 391, Academic Press, (2002)). Accordingly the methods and compositions of the present invention are not limited to a form of AR that is found in a particular organism, but are applicable to any form of AR that has been discovered or isolated, and to future forms thereof. Preferably, the methods and compositions of the present invention are for use with the human, chimpanzee, rat, and mouse forms of AR. Still more preferably, the methods and compositions of the present invention are for use with the human form of AR. The methods and compositions of the present invention are further applicable to recombinant forms of AR.

Currently, there is only one known AR isoform, although there are many naturally occurring mutants. Nevertheless, it would be understood that the methods of the present invention would be applicable to other isoforms of AR as are discovered or synthesized. In preferred embodiments, the amino acid sequence of the form of AR used corresponds identically to the amino acid sequence of the wild-type AR. However, in other embodiments of the invention, the sequence can comprise mutations. The mutations can, for example, be conservative or non-conservative. For example, a mutated residue of the mutant polypeptide can belong to the same amino acid class or sub-class as the corresponding residue of the wild type AR.

In a preferred embodiment of the present invention, the form of AR listed in the Swiss-prot database with accession number P10275 (see, e.g., us.expasy.org/cgi-bin/niceprot.p1?P10275) is suitable for forming crystals and for analysis. References to AR herein, unless otherwise indicated, are assumed to be to the sequence of human AR, identified by accession number P 10275. Where the sequence of an AR, or other nuclear receptor, is referred to and residues therein that “correspond” to those of human AR are identified, it is to be taken that the sequence of the receptor in question is to be aligned with that of human AR in a way that offers the greatest degree of homology.

It is further understood that the methods and compositions of the present invention are applicable to mutant forms of AR, from any organism, but preferably those found in humans. An example of a mutant form of AR is one linked to prostate cancer, the T877A mutant.

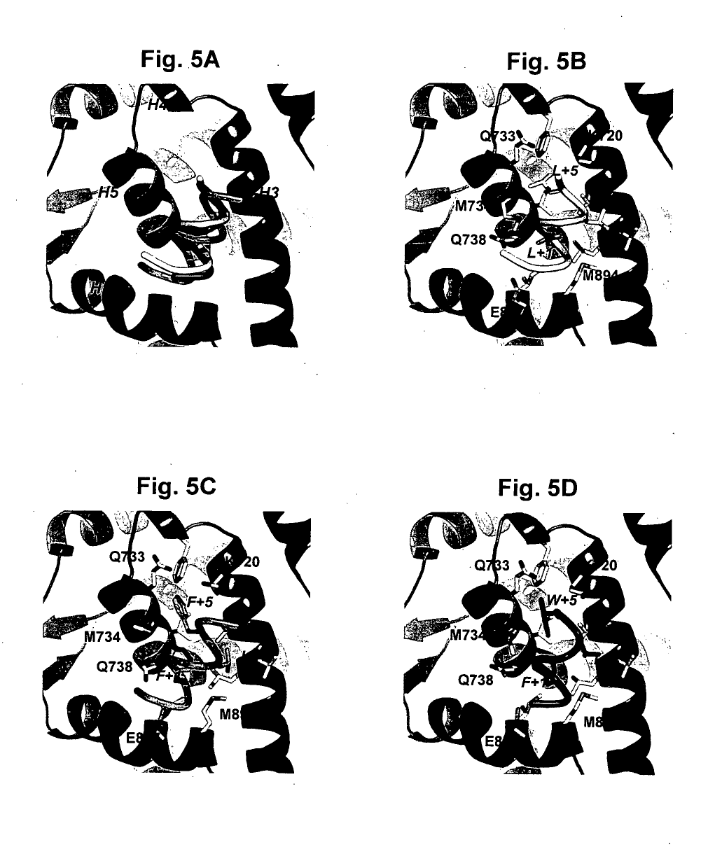

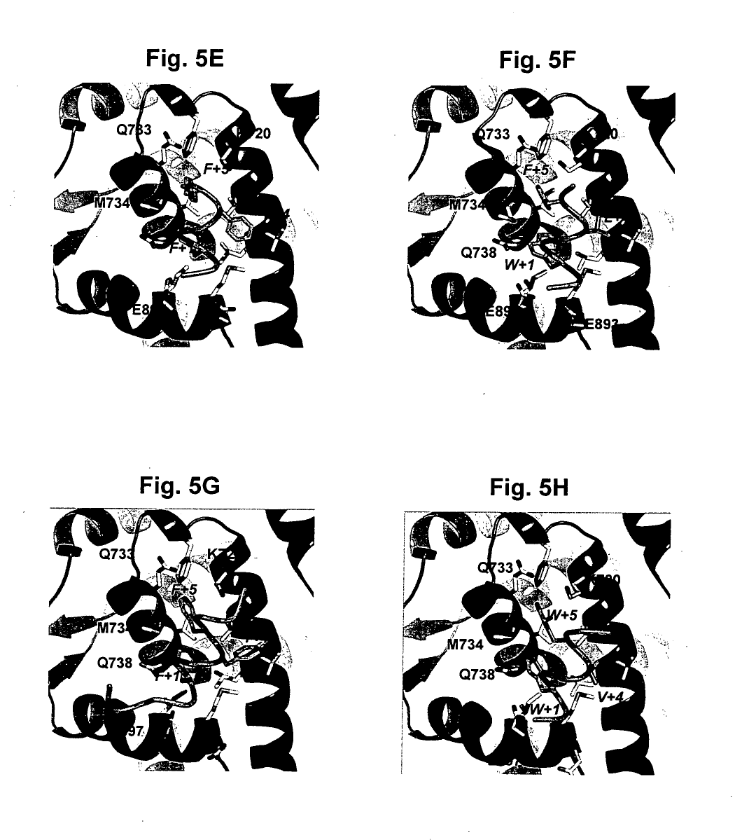

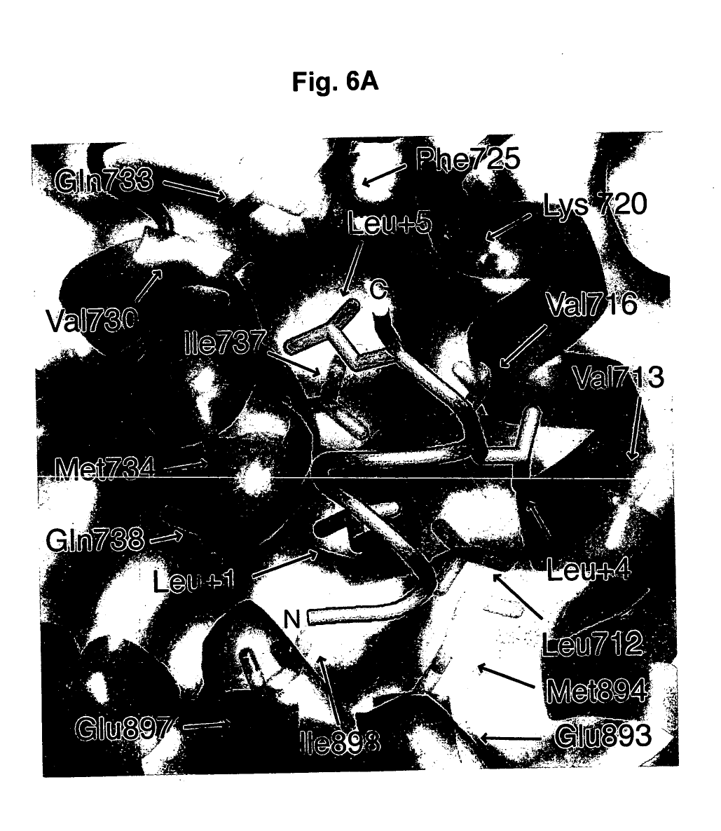

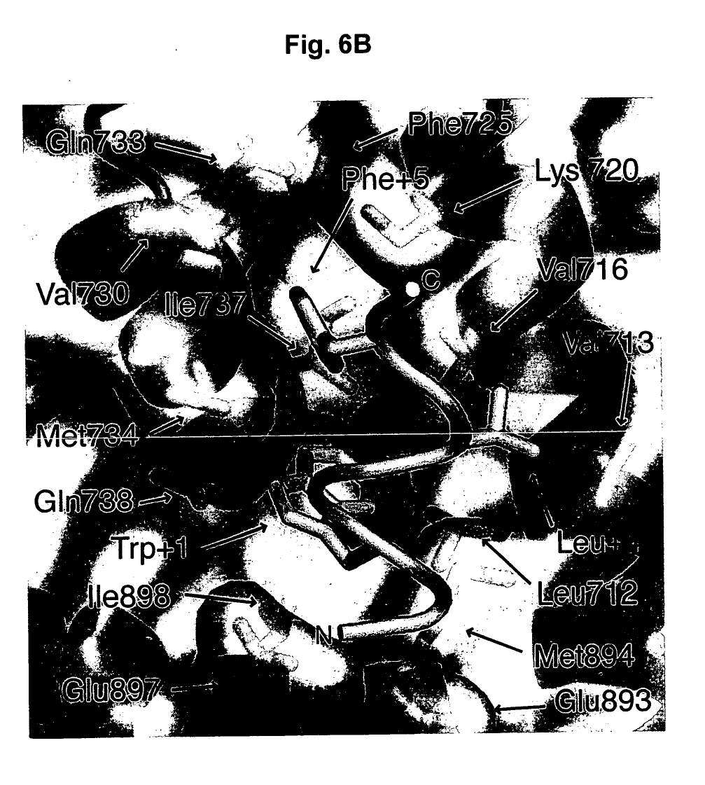

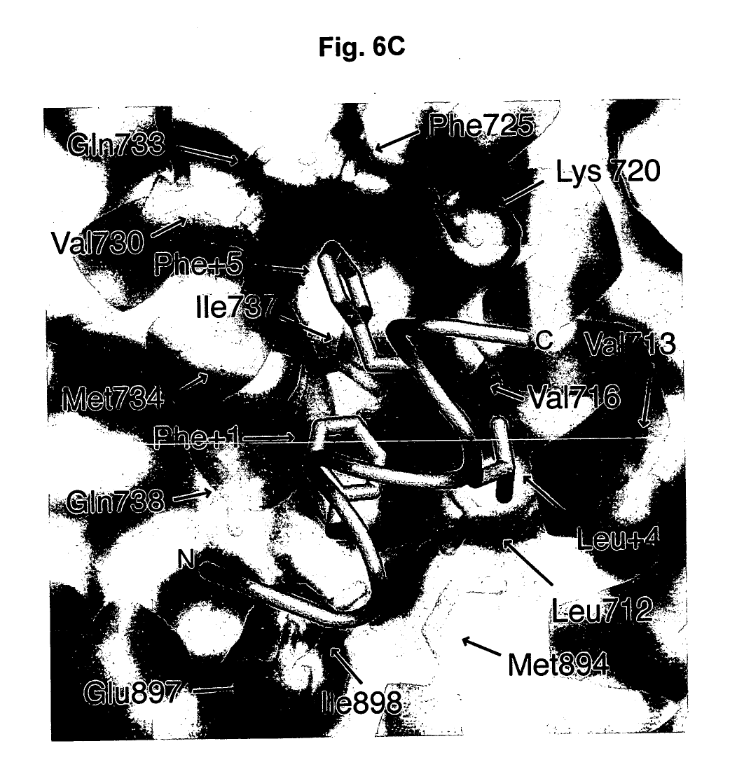

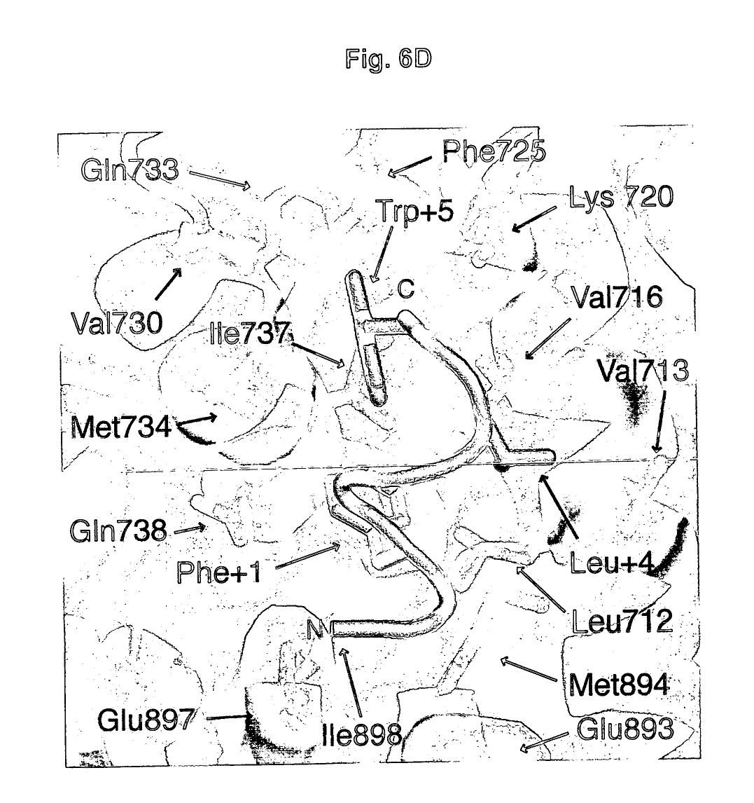

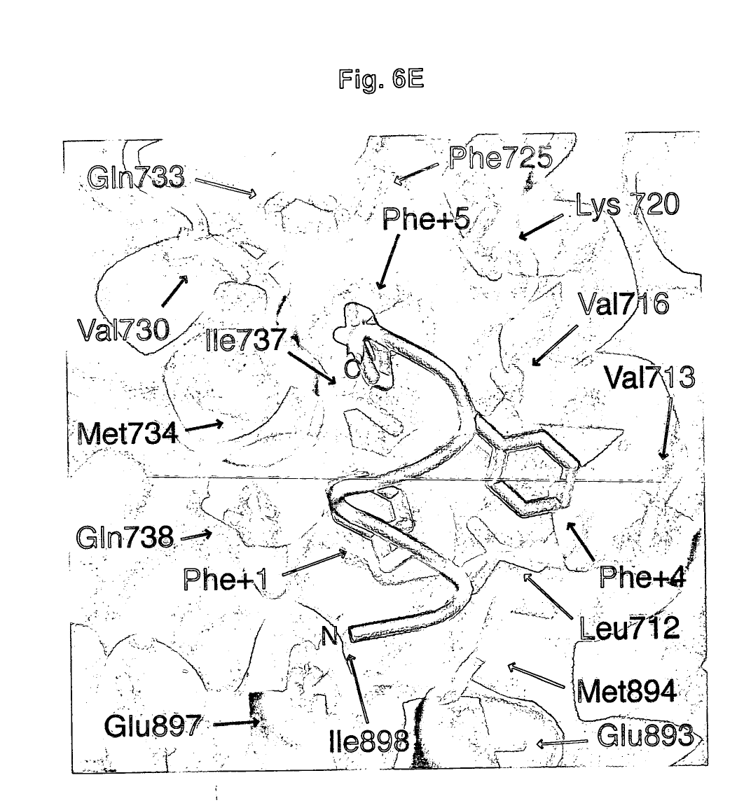

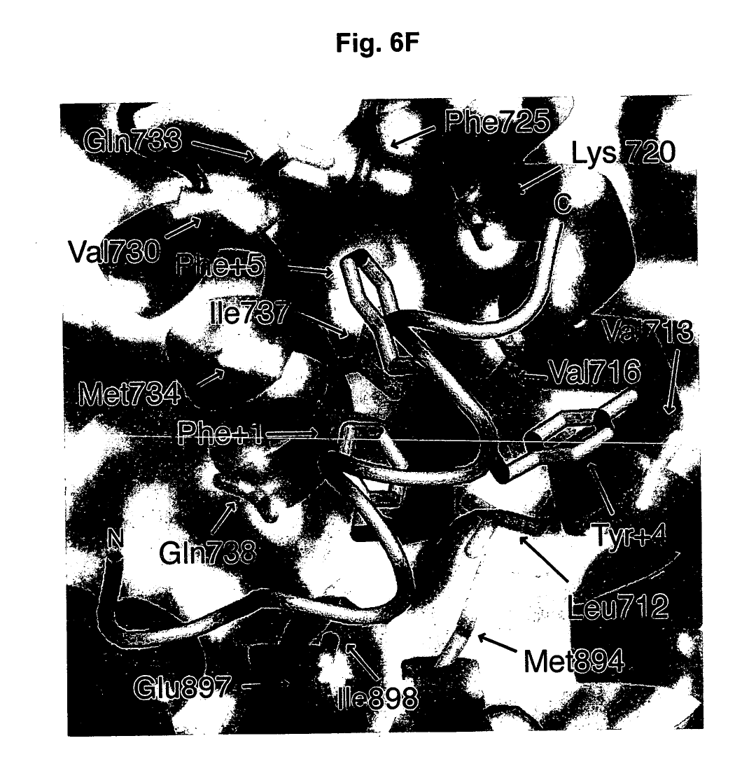

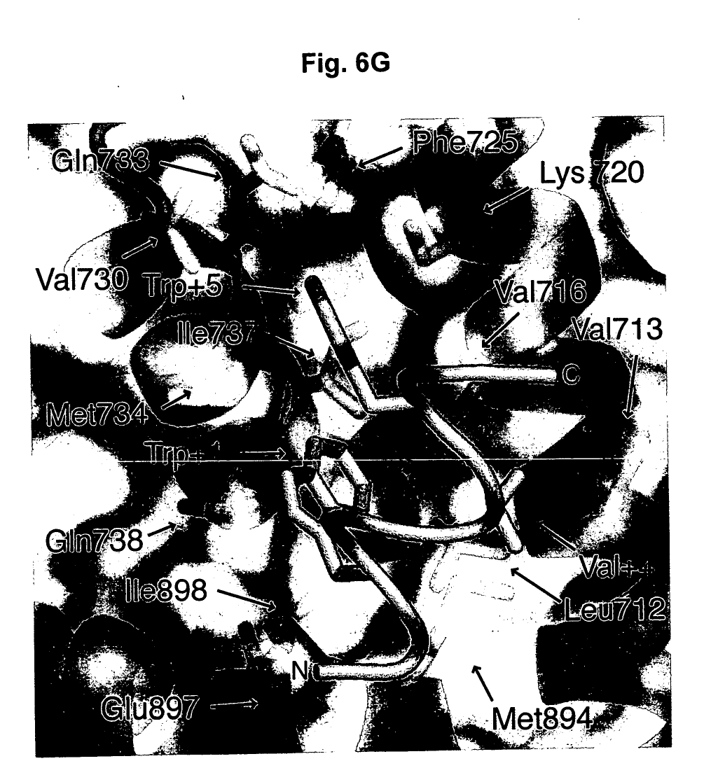

Certain residues forming the coactivator binding site on the androgen receptor were found to correspond to those positions described hereinabove for the human TR, as shown in FIG. 4. Accordingly, residues forming the coactivator binding site on AR correspond to the human AR residues of N-terminal helix 3 (Leu 712, Val 713, Val716, and Lys 720), helix 4 (Phe725), helix 5 (Gln 733, Met734, Ile737, and Gln738), helix 6 (Trp741), and C-terminal helix 12 (Glu 893, Met894, Glu 897, and Ile898). It has also been discovered that Val 730 interacts with a number of the coactivator peptides considered herein. These residues are illustrated in FIGS. 6A-6E, with the exception of H6 residue Trp 741, which is remote from contacting a coactivator molecule. FIGS. 6A-6E show computer-generated pictures of the coactivator binding site using atomic structural coordinates shown in Table 2 in the file identified as Table2_ARLBD_DHT_CRP.txt, presented on CD-R herewith, with a coactivator pentide bound thereto. Using the crystal structures of the present invention, certain residues within the AR coactivator binding site have been identified that are of particular importance in coactivator binding: Lys 720, Gln 733, Met 734, Gln 738, Met 894, and Glu 897 (see FIGS. 5A-F for an illustration of the position of these residues in the presence of a number of bound coactivator peptide molecules). Modifications to a molecule that enhance binding to, or interaction with, these residues would provide for an improved antagonist of coactivator binding.

Consistent with the structures of the present invention, conserved hydrophobic residues Val715 and Ala 719, and conserved residues Pro 723 and Ile 899, all of which correspond to coactivator binding site residues in other nuclear receptors, do not interact strongly with a coactivator peptide bound to AR. Such differences between coactivator binding to AR, and coactivator binding to other nuclear receptors has been revealed by obtaining crystal structures of coactivator bound AR, according to the present invention.

The AR coactivator binding site also differs from that of other nuclear receptor coactivator binding sites in that it can easily accommodate sequence motifs other than LXXLL in a manner that leads to favorable binding. Accordingly, with methods of the present invention, features of coactivator binding to AR, leading to design of potential inhibitors, have been explored with peptides that contain at least one core hydrophobic motif consisting of a sequence Z₁XXZ₂Z₃ (SEQ ID NO: 16), as further discussed hereinbelow. Specifically, where Z₁ and Z₃ are each independently F, L, W, or Y, and Z₂ is L, F, V, or Y, and X is any amino acid residue, details of pockets on the AR coactivator binding site are revealed. In particular, favored motifs include LXXLL, FXXLF, WXXLF, FXXFF (SEQ ID NO: 17), FXXLY (SEQ ID NO: 18), FXXYF (SEQ ID NO: 19), WXXVW (SEQ ID NO: 20), and FXXLW (SEQ ID NO: 21). Structures of the AR coactivator binding site obtained with such a peptide bound thereto reveal that this hydrophobic motif makes the principal interaction with the AR binding site. In the discussion herein, as well as the accompanying FIGs, residues of a peptide coactivator are numbered by reference to the first residue of the core hydrophobic motif, which is numbered+1. The residue immediately preceding that residue, i.e., outside of, and adjacent to, the motif, is numbered−1, and the one preceding that is numbered−2, and so on. The second residue of the core hydrophobic motif is numbered+2, and so on.

The peptides for use in identifying attributes of the coactivator binding site include coactivator-derived peptides, and coactivator-related peptides. Coactivator-derived peptides can be obtained from sequences found in coactivators including, but not limited to ARA70, GRIP1 box 1, GRIP1 box2, GRIP1 box3, and the N-terminal domain of AR itself. Coactivator-derived peptides preferably include various peptides that contain the sequence motifs LXXLL, FXXLF, and WXXLF. In particular, LXXLL is found in GRIP1 peptides, FXXLF is found in ARA70, and both FXXLF and WXXLF are found in the N-terminal domain of the AR LBD.

The structures of AR obtained with coactivator peptides reveal that the hydrophobic motifs Z₁XXZ₂Z₃, as described herein, bind in a manner analogous to those previously observed in other nuclear receptors that bind to LXXLL p160 coactivator motifs in the sense that generally, the core hydrophobic motif forms a short helix which binds in a groove formed by coactivator binding site helices 3, 4, 5, and 12 (see, e.g., FIG. 5A, depicting an overlap of CRP_{—}3 (FXXLF), green; CRP_{—}1 (LXXLL), yellow; CRP_{—}4 (FXXLW), violet).

The interactions between representative coactivator-related peptides and the AR coactivator binding site can further be summarized as follows. In particular, it is discovered that binding to the AR coactivator binding surface is predominantly hydrophobic in nature and is driven primarily by hydrophobic interactions with the amino acid residues at +1 and +5 of the hydrophobic Z₁XXZ₂Z₃ motif described herein.

Analysis of peptides containing a phenylalanine in the first (+1) and fifth (+5) positions of the hydrophobic motif demonstrate that Phe+1 binds in a wide pocket formed on the bottom by Ile898, and on the sides by the residues Met894, Gln738, Met734, Val716, and Leu712. By contrast, Phe+5 binds in a much narrower+5 pocket comprised of Ile737 on the bottom and Met734, Gln733, Val730, Phe725, Lys720, and Val716 on the sides. Thus, residue Met734 plays a role in defining two pockets. Phe 725 forms only a small part of the surface for the +5 pocket, specifically by forming the top of the +5 binding pocket and is probably a little too far away to make a strong interaction with a coactivator peptide. The bulk of the interactions in this pocket derive from Met734 and the aliphatic portion of Lys720, which interact with opposite faces of the benzyl ring of the coactivator residue Phe+5. Additionally, Leu+4 binds in a shallow cleft consisting of Val716, Val713, Leu712, and Met894. Residue Val 716 plays a role in defining three binding pockets.

Hydrophobic interactions between the LBD and hydrophobic residues of the other peptides that do not have phenylalanine groups in the +1 and +5 positions differ. Examination of a complex with a peptide containing the LXXLL motif indicates that Met734 makes a dramatic shift of about 2.5 Å toward the +1 pocket to accommodate the Leu+1 residue, thereby widening the +5 pocket. The position of the Met734 residue in such complexes also allows it to make a hydrophobic interaction with Trp+2 of the coactivator peptide.

The majority of differences between complexes of the AR coactivator binding site and various coactivator peptides lie in the nature of their polar interactions. In general, the main polar interactions between the AR LBD coactivator binding site involve the highly conserved “charge clamp” residues Lys720 and Glu897, which may interact with main chain carbonyl and amide groups at opposite ends of the coactivator peptide helix. Interactions with Lys720 generally take place with coactivator residues that are outside of the hydrophobic motif.

However, peptides with the FXXLF motif find it easiest to form interactions via their main chain amide nitrogens with the charge clamp residue, Glu 897. Comparisons with complexes of peptides that use other hydrophobic motifs reveal that the bound positions of the other peptides are skewed in a manner such that Glu897 is too far away to interact with peptide main chain atoms.

It has been further found that the formation of an interaction with Glu 897 is largely dependent on the length of the side chain at the +5 position of the peptide. Thus a Leu at +5 (in a peptide having, for example, the LXXLL motif), has a shorter sidechain than a Phe or a Tyr, and must reach over to fully make interactions with the hydrophobic +5 binding pocket, effectively pulling the rest of the peptide helix along with it. This causes a displacement of the entire peptide away from helix 12, toward helix 3, and a rotation about Met 734, thereby preventing interaction with Glu 897. On the other hand, a residue with a longer sidechain, such as Phe, at +5 of the motif is able to make the full set of interactions at the +5 site without causing a displacement of the peptide helix.

Accordingly, when designing inhibitors of coactivator binding with methods of the present invention, it is preferably to employ a peptide motif with Phe, or some other residue with an aromatic sidechain, such as Trp, or Tyr, in the +5 position in order to maxmize interactions in both the +1 and +5 binding pockets. This is notwithstanding the possibility that other residues in the hydrophobic motif of the coactivator, or those that flank the hydrophobic motif, may be able to interact with Glu897 For example, such an interaction may occur through a side chain hydroxyl group on a residue such as Ser-2.

Additional polar interactions may also occur when residues that can hydrogen bond, such as Trp, are present at +1 or +5 positions of the hydrophobic motif. For example, in a structure comprising the AR coactivator binding site and a coactivator peptide which contains the motif FXXLW, the charged hydrophylic nitrogen on the indole ring of Trp+5 forms a hydrogen bond with Gln 733. In general, the indole ring of the tryptophane side-chain inserts in the same pocket where the plenylalanine in the +5 position of FXXLF would sit. The important Gln 733 residue of hAR makes contact with that ring, thereby forming a very tight interaction. A similar set of interactions would be expected with a tyrosine residue in the +5 position of the coactivator peptide.

Similar polar interactions are seen in the structures that have AR bound to a coactivator peptide having Trp in the +1 position. In the +1 pocket, Trp+1 hydrogen bonds with Gln 738.

Still further polar interactions that are preferably obtained between a coactivator molecule and the AR coactivator binding site include hydrogen bonds with Glu893. This residue preferably hydrogen bonds with the main chain amide nitrogen of the −1 residue of a coactivator peptide.

Accordingly, using the structures and methods of the present invention, it is possible to deduce determinants of selectivity with respect to certain coactivator sequences. In particular, the disruption of main-chain polar interactions with Glu 897, as well as more favorable interactions at +5 with Met 734 represent key determinants of selectivity in AR with respect to motifs that have Phe vs. those that have Leu in the +5 position of the hydrophobic motif. For example, selectivity of FXXLF vs. LXXLL may thus be attributed to the F+5 position, rather than the F+1 position in FXXLF.

Sequence alignment reveals that, in positions corresponding to 734 in AR in most other nuclear receptors, Met is replaced by Val, Leu or Ile. The presence of a smaller branched hydrophobic residue such as valine, or leucine/isoleucine at this position would create a shallower, less favorable binding pocket for bulky aromatic residues at the +1 and +5 positions than would Met. Moreover, a smaller hydrophobic residue in the 734 position would not induce the rotation that is required to completely disrupt main-chain interactions with Glu 897 that occurs in LXXLL motifs. Therefore a significant difference in binding between a motif such as FXXLF and LXXLL would not be expected in those receptors that do not have Met in the 734 position.

Accordingly, structural analysis of AR coactivator binding sites to which a coactivator molecule is bound has revealed the mechanisms by which certain peptides bind the coactivator binding site and block coactivator binding, and hence transcriptional activity. Thereby, an understanding of coactivator binding has been achieved. Therefore, the coactivator binding site residues described hereinabove are useful in designing coactivator mimics that have broad application in the methods of the instant invention. Such “coactivator mimics” are peptides or polypeptides that mimic the coactivator binding site recognition area on the surface of a coactivator such that a “coactivator mimic” acts as a competitive inhibitor of coactivator binding to the coactivator binding site. Coactivator mimics can be used in an assay to determine receptor activity and hence the agonist or antagonist nature of a test compound, in that an agonist will permit a coactivator mimic to bind to the coactivator binding site, while an antagonist will prevent such binding. In addition, such coactivator mimics may have therapeutic utility when administered in combination with an agonist compound of the invention.

### EMBODIMENTS OF THE PRESENT INVENTION

It is to be understood that the methods and compositions of the present invention are applicable at several different levels, including: to members of the nuclear receptor family, such as androgens, estrogens, glucocorticoids, etc.; to members of a subfamily, or to variants that are found in different species, for example members of the androgen family including those in species such as human, chimpanzee, rat, and mouse, or for example members of the thyroid receptor family, such as TRα and TRβ; and to individual receptors, such as the human androgen receptor itself. Thus, where it is considered that a method is directed towards identifying compounds that bind the androgen receptor, it is also contemplated that such compounds could have activity against any other member of the androgen receptor subfamily, or to androgen receptor variants, as well as any other member of the nuclear receptor family.

Furthermore, it is contemplated that where test compounds that have been found to fit a model of the androgen receptor coactivator binding site are tested in an assay, that assay can involve binding of such compounds against either an androgen receptor or another member of the nuclear receptor family. Thus, the atomic structural models of the androgen receptor coactivator binding site presented herein can be used to model, and thus identify, compounds that not only bind the androgen receptor but may also bind other nuclear receptors.

Accordingly, one aspect of the invention is a method of identifying a compound that modulates (i.e., increases or decreases) nuclear receptor activity, comprising: modeling test compounds that fit spatially into a nuclear receptor coactivator binding site of interest using an atomic structural model of the androgen receptor ligand binding domain or portion thereof, screening the test compounds in an assay, for example a biological assay, characterized by binding of a test compound to the coactivator binding site, and identifying a test compound that modulates nuclear receptor activity, wherein the atomic structural model comprises atomic coordinates of human androgen receptor amino acid residues Leu 712, Val 713, Val716, Phe725, Gln 733, Met734, Ile737, Gln738, Trp741, Glu 893, Met894, Glu 897, and Ile898, preferably Gln 733, Met734, Gln738, Met894, and Glu 897, and additionally Lys 720, and additionally comprises coordinates of a coactivator bound to the coactivator bindin site. In a preferred embodiment, the nuclear receptor is an AR. It is to be understood that the atomic structural model may comprise the entire ligand binding domain, or portion thereof, and may also comprise coordinates of a ligand bound to the ligand binding domain.

The test compound can be an agonist and nuclear receptor activity is measured by binding of a coactivator or a compound that mimics a coactivator, to the coactivator binding site, as defined herein. In another embodiment, the test compound can be an antagonist and nuclear receptor activity is measured by the blocking of coactivator binding to the coactivator binding site. The screening is typically in vitro, and high throughput screening is preferable. Suitable test compounds can be designed, as is described herein, or can be obtained from a library of compounds, and include, by means of illustration and not limitation, small organic molecules, peptides and peptidomimetics. A library of compounds may be a combinatorial library, generated either in the laboratory, or virtually in a computer. The library of compounds may further be a commercially available selection of molecules that has been selected for a particular property, or for representative diversity of properties.

The methods described herein may also include the step of providing the atomic coordinates of the androgen receptor ligand binding domain, or portion thereof, to a computerized modeling system, prior to modeling. By providing is meant making available in electronic form so that one or more computer programs that run on the computerized modeling system are able to read the coordinates and perform manipulations on them such as, but not limited to, displaying them on a computer display.

Another embodiment of the present invention pertains to a method of identifying a compound that modulates ligand binding to a nuclear receptor, typically by binding to the coactivator binding site. This method comprises the steps of modeling test compounds that fit spatially into a nuclear receptor coactivator binding site of interest using an atomic structural model of the androgen receptor coactivator binding site or portion thereof, screening the test compounds in an assay characterized by binding of a test compound to the coactivator binding site, and identifying a test compound that modulates ligand binding to the nuclear receptor, wherein the atomic structural model comprises atomic coordinates of amino acid residues corresponding to residues of human androgen receptor Leu 712, Val 713, Val716, Phe725, Gln 733, Met734, Ile737, Gln738, Trp741, Glu 893, Met894, Glu 897, and Ile898, preferably Gln 733, Met734, Gln738, Met894, and Glu 897, and additionally Lys 720. In a preferred embodiment, the nuclear receptor is ER, TR, GR or PR. The screening is typically in vitro such as by high throughput screening. Suitable test compounds can be designed or obtained from a library of compounds and include, by means of illustration and not limitation, small organic molecules, peptides, and peptidomimetics. The test compounds can be either agonists or antagonists of ligand binding. Compounds of particular interest fit spatially and preferentially into the coactivator binding site.

The invention also includes methods for identifying key residues within the coactivator binding sites of nuclear receptors. The methods involve examining the surface of a nuclear receptor of interest to identify residues that modulate ligand and/or coactivator binding. The residues can be identified by homology to the key residues on the coactivator binding site of human AR described herein. A preferred method is alignment with the residues of any nuclear receptor corresponding to (i.e., equivalent to) human AR residues of Leu 712, Val 713, Val716, Lys 720, Phe725, Gln 733, Met734, Ile737, Gln738, Trp741, Glu 893, Met894, Glu 897, and Ile898, preferably Gln 733, Met734, Gln738, Met894, and Glu 897. Overlays and superpositioning with a three-dimensional model of a nuclear receptor coactivator binding site, or a portion thereof, that contains these or corresponding residues, also can be used for this purpose. For example, three-dimensional structures of TR, GR and PR LBD's can be used for this purpose. Exemplary nuclear receptors identifiable by homology alignment include normal nuclear receptors, or proteins structurally related to nuclear receptors found in humans, natural mutants of nuclear receptors found in humans, normal or mutant receptors found in animals, as well as non-mammalian organisms such as pests or infectious organisms, or viruses.

Alignment and/or modeling also can be used as a guide for the placement of mutations on the coactivator binding site surface to characterize the nature of the site in a cellular environment. Selected residues are mutated to preserve global receptor structure and solubility, and to permit helix 12 to unwind or fall away, in the case of an antagonist. Mutants can be tested for ligand binding as well as the relative change in strength of the coactivator binding interaction. Ligand-dependent coactivator interaction assays also can be tested for this purpose, such as those described herein.

In particular, the present invention relates to the structural and functional effects on the androgen receptor's coactivator binding site, of the binding of different coactivator molecules. As described in the Examples hereinbelow, analysis of atomic models derived from cocrystals, reveals the structure of the human androgen receptor coactivator binding site co-crystallized with a peptide molecule bound to the coactivator binding site and the agonist, DHT. The peptide comprises a GRIP1 NR Box 3 peptide sequence (i.e., a peptide derived from the NR Box 3 region of the p160 coactivator GRIP1), or an ARA70 peptide sequence, or a hydrophobic motif similar to a nuclear receptor box. The Examples provide the crystal structure of the hAR LBD bound to DHT and the coactivator molecules: RETSEKFKLLFQSYN (2.3 Å resolution); KENALLRYLLDKDD (2.07 Å resolution); SSRGLLWDLLTKDSR (1.6 Å resolution); SRWQALFDDGTDTSR (2.2 Å resolution); SSRFESLFAGEKESR (1.45 Å resolution); SSKFAALWDPPKLSR (1.8 Å resolution); SRFADFFRNEGLSGSR (2.2 Å resolution); SRWAEVWDDNSKVSR (2.1 Å resolution); and SSEVTGMRFRDLFSR (1.9 Å resolution); SSNTPRFKEYFMQSR (1.6 Å resolution) That is, the crystals diffract with a resolution that is as good as 1.6 Å.

The invention is also applicable to generating new compounds that distinguish between nuclear receptor isoforms. Such a capability can facilitate generation of either tissue-specific or function-specific compounds. For instance, although GR subfamily members usually have one receptor encoded by a single gene, there are exceptions to be found in the larger nuclear receptor super-family. For example, there are two PR isoforms, A and B, translated from the same mRNA by alternate initiation from different AUG codons. There are two GR forms, one of which does not bind ligand.

The present invention also includes a method for identifying a compound capable of selectively modulating nuclear receptor activity. The method comprises the steps of modeling test compounds that fit spatially and preferentially into the coactivator binding site of a nuclear receptor of interest using an atomic structural model of an androgen receptor comprising coordinates of a coactivator molecule bound to the coactivator binding site, screening the test compounds in an assay for nuclear receptor activity characterized by preferential binding of a test compound to the coactivator binding site of a nuclear receptor, thereby identifying a test compound that selectively modulates the activity of a nuclear receptor. Such receptor-specific compounds are selected that exploit differences between the coactivator binding sites of one type of nuclear receptor versus a second type of nuclear receptor.

The receptor-specific compounds of the invention preferably interact with conformationally constrained residues of the coactivator binding site that are conserved among one type of nuclear receptor relative to a second type of nuclear receptor. “Conformationally constrained” is intended to refer to the three-dimensional structure of a molecule, or moiety thereof, in which certain rotations of groups about its bonds are hindered by various local geometric and physico-chemical constraints. Conformationally constrained structural features of a coactivator binding site include residues that have their natural flexible conformations fixed by various geometric and physico-chemical constraints, such as conformation of the local backbone, orientation of local side chain(s), and topological constraints arising from aspects of secondary, tertiary, and quaternary structure. These types of constraints can be exploited to restrict positioning of atoms involved in receptor-coactivator recognition and binding. Such conformationally constrained residues can be identified by one of ordinary skill in the art, using the coordinates of the structures presented on CD-R herewith.

The atomic coordinates of a compound that fits into the coactivator binding site also can be used for modeling to identify compounds or fragments that bind the site. Thus, the present invention also provides for a computational method that uses three dimensional models of an androgen receptor derived from a crystals of an androgen receptor, preferably cocrystals of an androgen receptor and a coactivator molecule. Such models can be said to be experimentally derived, as opposed derived computationally, such as by homology modeling. Generally, the computational method of designing a nuclear receptor coactivator involves determining which amino acid or amino acid residues of a nuclear receptor coactivator binding site interact with at least one moiety of the coactivator, by using a three dimensional model of a crystallized protein comprising a nuclear receptor coactivator binding site with a bound coactivator. The method further comprises selecting at least one chemical modification of the moiety to produce a second moiety that either decreases or increases an interaction between the interacting amino acid residue and the second moiety when compared to the interaction between the interacting amino acid residue and the original moiety. Such a modification can be carried out virtually, by using a computer modeling program as further described herein, or in the laboratory, as applied to a sample of the molecule. In the instant invention, crystal structures of the AR with coactivator-related peptides and with coactivator-derived peptides, have shown that amino acid residues that correspond to AR Leu 712, Val 713, Val716, Phe725, Gln 733, Met734, Ile737, Gln738, Trp741, Glu 893, Met894, Glu 897, and Ile898, preferably Gln 733, Met734, Gln738, Met894, Glu 897, and Lys 720, interact with at least one chemical moiety on a coactivator molecule, and therefore that coactivator moieties that interact with such residues should be considered for derivatization.

This computational method may further comprise quantifying a change in interaction between the interacting amino acid and the ligand after modification of the first moiety. The modification can either enhance or reduce a hydrogen bonding interaction, a charge interaction, a hydrophobic interaction, a van der Waals interaction, or a dipole interaction between the second moiety and the interacting amino acid, as compared to the interaction between the first moiety and the interacting amino acid. Chemical modifications will often enhance or reduce interactions between an atom of a coactivator binding site amino acid and an atom of a coactivator. Steric hindrance will be a common means of changing the interaction between the coactivator binding site binding cavity and the activation domain. Typical substituents are hydrophobic groups, including by way of example and not limitation, alkyl groups such as ethyl, propyl, isopropyl, etc., and aromatic groups such as benzyl, etc.

It is to be further understood that the atomic structural coordinates of the present invention may be used to assist in structure determination of another member of the nuclear receptor family, using methods described herein, and also those of homology modeling and other methods familiar to one of ordinary skill in the art of protein modeling and design.

For use in conjunction with the present invention, the LBD comprising the coactivator binding site of a nuclear receptor such as AR can be expressed, crystallized, and its three dimensional structure determined with a ligand and coactivator bound (either using crystal data from the same receptor or a different receptor or a combination thereof), and computational methods used to design ligands to its LBD.

Design, Preparation and Purification of Peptides that Interact with the Androgen Receptor Coactivator Binding Site

Peptide coactivators for use with methods, complexes, crystals, and compositions of the present invention preferably comprise a motif for interaction with the coactivator binding domain of AR. Thus, such peptide coactivators for use in conjunction with the present invention preferably comprise the sequence motif Z₁XXZ₂Z₃, wherein Z₁ and Z₃ are each independently F, L, W, or Y, and Z₂ is L, F, V, or Y, and X is any amino acid residue. In a preferred embodiment, Z₁ and Z₃ are each independently F, L or W, and Z₂ is L or F, and X is any amino acid residue. In a still more preferred embodiment, Z₁ and Z₃ are each independently F or W, and Z₂ is L, and X is any amino acid residue. It is also preferred that Z₂ is not W. By using peptides comprising such a sequence motif, details of interaction with binding pockets on the AR coactivator binding site are revealed. In particular, favored motifs include LXXLL, FXXLF, WXXLF, FXXFF, FXXLY, FXXYF, WXXVW, and FXXLW. Accordingly, such peptides preferably comprise a sequence such as LXXLL, FXXLF, WXXLF, FXXFF, FXXLY, FXXYF, WXXVW, and FXXLW.

The motif LXXLL is found in the naturally occurring coactivators GRIP, p160, SRC-1, TIF2, and Receptor-associated Coactivator 3 (RAC3); the motif FXXL(F/Y) occurs in the naturally occurring coactivators ARA70, ARA54, ARA55, FHL2, and also in the N-terminal domain of AR; the motif LXXXIXXX(I/L) is found in the naturally occurring corepressors SMRT, NCoR; and the motif WXXLF (SEQ ID NO: 22) (specifically WHTLF (SEQ ID NO: 23)) is also found in the N-terminal domain of AR.

Peptide coactivators of the present invention comprising the sequence motif Z₁XXZ₂Z₃, wherein Z₁ and Z₃ are each independently F, L, W, or Y, and Z₂ is L, F, V, or Y, and X is any amino acid residue, are preferably exactly 5 amino acid residues in length, are still more preferably from 6 to 10 residues in length, are even more preferably from 111 to 15 residues in length, and may further be from 16 to 20 amino acid residues in length. In general the peptide coactivators of the present invention are from about 6 to about 15 amino acid residues in length. It is especially preferable that peptide coactivators for use with the present invention are 14 or 15 amino acid residues in length. It is further consistent with the present invention that the peptide coactivators are capped at one or both termini by a protecting group, such as Fmoc, as would be understood by one of ordinary skill in the art.

Peptides of the present invention may further include peptides comprising the sequence motif Z₁XXZ₂Z₃, as described herein, wherein X can be a non-naturally occurring amino acid, or a D-amino acid. Non-naturally occurring amino acids for use in peptides of the present invention are preferably those that are commercially available, and include amino acids that have aliphatic and aromatic side-chains other than the side-chains on the amino acids found in nature, and also include amino acids that have derivatized side-chains. Such derivatized side-chains preferably include side chains of naturally occurring amino acids that have been substituted with one or more halogens, one or more hydroxy groups, or one or more alkyl groups, and may further include side chains wherein one or more carbons has been replaced by a heteroatom.

Any method of identifying peptides, and particularly those methods that select for peptides that bind at the coactivator binding site of a nuclear receptor such as the androgen receptor, may be used in conjunction with the present invention. See, for example, Hyde-DeRuyscher, R., et al., “Detection of small-molecule enzyme inhibitors with peptides isolated from phage-displayed combinatorial peptide libraries”, Chem. Biol., 7:17-25, (1999), incorporated herein by reference in its entirety. In particular, in conjunction with the present invention, to identify peptides that interact with the androgen receptor it is preferred to use the the AR ligand binding domain, and phage-display techniques similar to those described in: Sparks, A. B., Adey, N. B., Cwirla, S., Kay, B. K., in Phage Display of Peptides and Proteins, A Laboratory Manual, eds. Kay, B. K., Winter, J., and McCafferty, J., (Academic, San Diego), pp. 227-253, (1996), which is incorporated herein by reference in its entirety.

Additional methods of identifying peptides that bind to the androgen receptor are known in the art and may also be used in conjunction with the methods of the present invention. One appropriate method is described in International Patent Application Publication No. WO 98/19162, which is incorporated herein by reference. This method is directed to the identification of compounds in a compound library. The compounds, including biopolymers such as peptides, modulate the biological activity of a target receptor protein, even when ligands that modulate that activity through binding to the receptor are not already known. Once identified, such compounds can then be used as “leads” in a drug discovery program, i.e., they can be used as a starting point for the design of analogues which can in turn be synthesized and tested for activity against the protein. Accordingly, such methods may appropriately be used to discover compounds that potentially bind to the coactivator binding site of a nuclear receptor such as AR.

As disclosed in International publication No. WO 98/19162, it is believed that those members of a combinatorial library, especially a biopolymer library, which bind to a target protein such as AR, or the AR LBD, and have a biologically significant binding activity will bind preferentially to the sites at which the target protein interacts with its natural binding partners. That is, it is expected that such compounds will bind preferentially to the binding sites, as opposed to randomly, with equal probability, over the entire surface of the target protein. The method described in WO 98/19162 comprises three general steps: (1) Screen at least one potential surrogate combinatorial library for members (preferably peptides or nucleic acids) that binding to a target protein such as AR, or AR LBD, and hence are capable of use as surrogates for the unknown ligand in the subsequent steps (2) and (3); (2) Screen at least one complementary library, preferably another combinatorial library, (which is not limited to, and need not even include peptides or nucleic acids) for compounds which inhibit the binding of one or more surrogates to the target protein (e.g., peptides or nucleic acids which bind to AR or the AR LBD); and (3) Determine whether an inhibitory compound discovered in step (2) modulates the biological activity of the target protein.

The peptides described herein may be chemically synthesized in whole or in part using techniques, that are well-known in the art (see, e.g., Creighton, Proteins: Structures and Molecular Principles, W.H. Freeman & Co., NY, (1983)). Alternatively, methods that are well known to those of ordinary skill in the art can be used to construct expression vectors containing the native or mutated polypeptide coding sequence and appropriate transcriptional/translational control signals. These methods include in vitro recombinant DNA techniques, synthetic techniques and in vivo recombination/genetic recombination. See, for example, the techniques described in Maniatis, et al., Molecular Cloning: A Laboratory Manual, Cold Spring Harbor Laboratory, NY, (1989), and Ausubel, et al., Current Protocols in Molecular Biology, Greene Publishing Associates and Wiley Interscience, NY, (1989).

As discussed hereinabove, preferred peptides for use with the present invention can be referred to as “coactivator-derived” or “coactivator-related” according to whether or not flanking residues surrounding a coactivator binding motif are also found in the sequence of a known physiologically active coactivator molecule. One aspect in which coactivator-related and coactivator-derived peptides differ from one another is in the form of the binding curve that is obtained when they are assayed for their ability to bind to a receptor. A co-activator-related peptide typically gives a conventionally-shaped curve in which the slope of binding affinity against concentration is shallow. By contrast, coactivator-derived peptides give a titration curve that is sharp, and in which the slope approaches closely to the vertical. Such a distinction has been attributed to “cooperativity”, whereby the portions of the peptide sequence of the coactivator-derived peptide that are outside the motif, also interact with the receptor to promote binding.

Preparation and Purification of Protein and Complexes

When a particular domain is isolated from the remainder of the protein, its separate domain function is usually preserved. A number of methods, known to one of ordinary skill in the art, may be applied to obtaining a sample of a particular domain of a protein. Using protein chemistry techniques, a modular domain can be separated from the parent protein. Using molecular biology techniques, each domain of a protein can usually be separately expressed with its original function intact. Alternatively, chimerics of two different nuclear receptors can be constructed, wherein the chimerics retain the properties of the individual functional domains of the respective nuclear receptors from which the chimerics were generated.

Nuclear receptor protein samples for crystals and assays described herein can be produced using expression and purification techniques described herein and known to one of ordinary skill in the art. For example, high level expression of nuclear receptor LBD's can be obtained in suitable expression hosts such as E. coli. LBD's that have been expressed in E. coli, for example, include the ERα LBD and other nuclear receptors, including GR and PR. Yeast and other eukaryotic expression systems can be used with nuclear receptors that bind heat shock proteins because these nuclear receptors are generally more difficult to express in bacteria. Representative nuclear receptors, or their ligand binding domains, have been cloned and sequenced: human ER (see Seielstad, et al., Molecular Endocrinology, 9(6):647-658, (1995)), human GR, and human PR.

Coactivator proteins for use with the present invention can also be expressed using techniques known to one of ordinary skill in the art. In particular, members of the p 160 family of coactivator proteins that have been cloned and/or expressed previously, include SRC-1, AIB1, RAC3, p/CIP, and GRIP1 and its homologues TIF 2 and NcoA-2. A preferred method for expression of coactivator protein such as GRIP1 is to express a fragment that retains transcriptional activation activity using the “Song and Fields” method (also referred to as the “yeast 2-hybrid” method, see Hong, et al. Mol. Cell. Biol., 17:2735-44, (1997), and Proc. Natl. Acad. Sci. USA, 93(10):4948-52, (1996)).

The nuclear receptor, or coactivator proteins can be expressed alone, as fragments of the mature or full-length sequence, or as fusions to heterologous sequences. For example, AR can be expressed without any portion of the DBD or amino-terminal domain. Portions of the DBD or amino-terminus can be included if further structural information with amino acids adjacent the LBD is desired. Generally, for AR, the LBD used for crystals will be less than 500 amino acids in length. Preferably, the AR LBD will be at least 200 amino acids in length and most preferably at least 240 amino acids in length. For example the LBD used for crystallization can comprise amino acids spanning residue positions from 669 to 918 of AR. However, it is to be understood that the cocrystals of the present invention may also be formed from portions of the AR that are capable of folding into the LBD, and thus may be longer or shorter than the sequence that runs from residue 669 to residue 918. In particular, such portions may be shorter or longer by up to about 5 amino acid residues, or up to about 10 amino acid residues, or up to about 20 amino acid residues, or up to about 50 amino acid residues, wherein the sequence may be truncated at one or both ends relative to the portion that starts at residue 669 and ends at residue 918.

Typically the nuclear receptor LBD's are purified to homogeneity to facilitate crystallization. Purity of LBD's can be measured with sodium dodecyl sulfate polyacrylamide gel electrophoresis (SDS-PAGE), mass spectrometry (MS), and hydrophobic high performance liquid chromatography (HPLC). The purified LBD for crystallization should be at least 97.5% pure, preferably at least 99.0% pure, and more preferably at least 99.5% pure.

Purification of an unliganded sample of a nuclear receptor for use with the present invention can be obtained by conventional techniques, such as hydrophobic interaction chromatography (HIC), ion exchange chromatography, and heparin affinity chromatography. To achieve higher purification for improved quality crystals of nuclear receptors, such as the androgen receptor, the receptors can be ligand-shift-purified using a column, such as an ion exchange or hydrophobic interaction column, that separates the receptor according to charge, and then bind the eluted receptor with a ligand, especially an agonist. The ligand induces a change in the receptor's surface charge such that the liganded receptor elutes at a different position than the unliganded receptor. Usually, saturating concentrations of ligand are used in the column, and the protein can be pre-incubated with the ligand prior to passing it over the column. The structural studies detailed herein indicate the general applicability of this technique for obtaining super-pure nuclear receptor LBD's for crystallization.

Purification can also be accomplished by use of a purification handle or “tag,” such as a histidine amino acid engineered to reside on one end of the protein, such as the N-terminus, and then using a nickel or cobalt chelation column for purification (see Janknecht, Proc. Natl. Acad. Sci. USA, 88:8972-8976, (1991)). Typically, purified LBD, such as AR LBD, is equilibrated at a saturating concentration of ligand at a temperature that preserves the integrity of the protein. Ligand equilibration can be established between about 2 and about 37° C., although the nuclear receptors tend to be more stable in the 2-20° C. range.

Cocrystals of AR with a Ligand and a Coactivator

The cocrystals of the present invention comprise an AR ligand binding domain, or portion thereof, and a molecule bound to the coactivator binding site. Preferably the crystals of the present invention also include a ligand bound to the ligand binding domain.

The cocrystals of the present invention are preferably prepared by a method that comprises, prior to crystallization, binding a ligand to the AR ligand binding domain, followed by binding a coactivator to the AR coactivator binding site, for example, by incubating an AR-ligand complex in a molar excess of coactivator peptide for several hours.

In order to select a ligand for co-crystallization with an AR ligand binding domain, small organic molecules and peptides can be assayed for binding to the ligand binding domain and coactivator binding sites of a nuclear receptor of interest by any number of methods, including assays described herein. For co-crystallization with a ligand that binds the ligand binding domain, alone or in conjunction with a peptide that binds to the coactivator binding site, various concentrations of ligands containing a sequence that binds to a coactivator binding site of a nuclear receptor of interest can be used in microcrystallization trials, and the appropriate complexes selected for further crystallization.

Ligands for use in the present invention for forming cocrystals of the AR LBD are preferably small organic molecules. Still more preferably, such organic molecules are steroids. Preferably a ligand is a hormone such as a known androgen that binds the AR LBD. Still more preferably the ligand is 5α-dihydrotestosterone (“DHT”), or an analog thereof, such as methyltrienolone (R1881). The present invention also encompasses ligands such as 1,1-dichloro-2,2-bis(p-chlorophenyl)ethylene (p,p′-DDE). The present invention further comprises ligands that are analogs or derivatives of the foregoing compounds, such as may be obtained by methylating, ethylating, or otherwise substituting one or more groups on the molecules that do not significantly disrupt the molecule's binding attributes.

As described in the Examples presented hereinbelow, AR LBD's are co-crystallized with a molecule comprising a coactivator such as a peptide bound to the coactivator binding site, and with a ligand bound to the LBD. In each case, the cocrystal structure it is preferable that the crystal structure is refined to a resolution of better than 2.5 Å; it is even more preferable that the crystal structure is refined to a resolution of better than 2.0 Å.

Crystallization is preferably carried out with coactivator-related peptides whose sequence comprises a motif Z₁XXZ₂Z₃, wherein Z₁ and Z₃ are each independently F, L, W, or Y, and Z₂ is L, F, V, or Y, and X is any amino acid residue. In a preferred embodiment, Z₁ and Z₃ are each independently F, L or W, and Z₂ is L or F, and X is any amino acid residue. In a still more preferred embodiment, Z₁ and Z₃ are each independently F or W, and Z₂ is L, and X is any amino acid residue. It is also preferred that Z₂ is not W. In particular, favored motifs include LXXLL, FXXLF, WXXLF, FXXFF, FXXLY, FXXYF, WXXVW, and FXXLW. Such peptides are preferably from about 6 to about 20 residues in length, and are preferably 14 or 15 residues long. Such peptides include, but are not limited to peptides whose sequences are: SSRFESLFAGEKESR; SSKFAALWDPPKLSR; SRFADFFRNEGLSGSR; SRWQALFDDGTDTSR; SSRGLLWDLLTKDSR; SSEVTGMRFRDLFSR (SEQ ID NO: 24); SRWAEVWDDNSKVSR; and SSNTPRFKEYFMQSR.

For the purposes of the present invention, crystallization is also preferably carried out with a peptide whose sequence comprises a portion of the sequence of a naturally occurring coactivator. Still more preferably, the present invention comprises crystals obtained with coactivator peptides derived in the following manner: RETSEKFKLLFQSYN from ARA70; KENALLRYLLDKDD from GRIP1 Box3; KHKILHRLLQDSS (SEQ ID NO: 25), from GRIP1 Box2; and HKKLLQLLT, from RAC3.

Accordingly, the present invention provides for cocrystals comprising an AR ligand binding domain with a ligand bound to the ligand binding domain, and a molecule bound to the coactivator binding site. Preferably, the cocrystal structure is refined to a resolution better than 3.6 Å, i.e., having a resolution value less than 3.6 Å. More preferably the cocrystal structure is refined to better than 3.4 Å, 3.2 Å, 3.0 Å, 2.8 Å, 2.6 Å, 2.4 Å, 2.2 Å, even more preferably to a resolution better than 2.0 Å. Still more preferably the structure is refined to better than 1.5 Å. Resolution is crystal dependent—in my case:

Crystals are preferably made from purified nuclear receptor LBD's, for example those that are expressed by a cell culture, such as E. coli, a preferred expression system frequently used by those of ordinary skill in the art.

For crystallization trials with the AR LBD, the hanging drop vapor diffusion method is preferred. In other embodiments of the present invention, the “sitting drop” method can be employed. Preferably crystals of the present invention are made with hanging drop methods such as those described herein. Regulated temperature control is desirable to improve crystal stability and quality. Temperatures between about 4 and about 25° C. are generally used and it is often preferable to test crystallization over a range of temperatures. Conditions of pH, solvent and solute components and concentrations, and temperature can be adjusted, for instance, as described in the Examples hereinbelow. The crystals are subjected to vapor diffusion and bombarded with X-rays to obtain X-ray diffraction patterns according to standard procedures familiar to one of ordinary skill in the art. In the hanging drop method, improved crystal size and quality suitable for X-ray diffraction analysis can be obtained through microseeding techniques, such as seeding of prepared drops with microcrystals of the complex, as would be familiar to one of ordinary skill in the art.

Preferably, different cocrystals for AR are made separately using different types of coactivator molecules, such as protein fragments, fusions, small peptides, or small organic molecules. The types of coactivator molecules preferably contain NR-box sequences suitable for binding to the coactivator binding site, or derivatives of NR-box sequences. Other molecules are preferably used in co-crystallization, such as small organic molecules that bind to the hormone binding site. By obtaining cocrystals that utilize different types of coactivator molecules, it is possible to glean important information about coactivator binding and AR function.

Crystallization of AR with ligand, and different coactivator peptides, containing coactivator binding motifs such as LXXLL, FXXFF, FXXLW, WXXVW, WXXLF, and FXXLF, allows one of ordinary skill in the art to understand the structural details of how the aromatic rich co-activator motif, FXXLF adapts to the LXXLL binding site. Specifically, the coactivators chosen include molecules that comprise the LXXLL motif of p160 coactivators. In particular, the interaction of the p160 coactivator GRIP1 box2 and GRIP1 box3 peptides with AR LBD have been elucidated by co-crystallization. A crystal containing a peptide whose sequence is derived from GRIP1, including the box2 motif, diffracted to 1.66 Å; a further crystal containing a peptide whose sequence is derived from GRIP1, including the box 3 motif, diffracted to 2.07 Å. The present invention also defines the structural basis for the interaction of AR with the FXXLF motif present in the coactivator ARA70. Co-crystals of ARA70 peptide with AR LBD have been grown and a complete data set was obtained at 2.3 Å resolution. Heavy atom substitutions can be included in the LBD and/or a co-crystallizing molecule for facilitating imaging. Heavy atom derivatives can be used to obtain initial phase estimates which can then be used to solve the structure, a process that typically is not necessary when there are molecular replacement models available.

Accordingly, the cocrystals of the present invention may be used to define the structural and molecular basis for the interaction of AR with a coactivator molecule, and to identify the determinants of specificity of these interactions.

Methods of Structure Determination and Refinement

Diffraction data for the crystals of the present invention can be measured at a radiation source, preferably a synchrotron source such as the Advance Light Source at the Lawrence Berkeley National Laboratory, or the Stanford Synchrotron Radiation Laboratory (SSRL), using conditions that are accessible to one of ordinary skill in the art. Structural information for new complexes can be determined by molecular replacement using the structure of the AR LBD determined herein. The structure is refined following standard techniques known in the art.

Various methods of structure determination and refinement may be used to derive the atomic coordinates for the cocrystal structures of the present invention, as would be understood by one of ordinary skill in the art. For example, the images can be processed with a program such as DENZO and scaled with SCALEPACK (both of which are attributed to Otwinowski, et al., see, e.g., Methods Enzymol., 276:307-326, (1997)) using the default parameters such as the −3σ cutoff. In some situations, initial efforts to determine the structure of a complex can utilize a low resolution data set (such as at a resolution of about 3.1 Å or worse). Other approaches that can be used include a self-rotation search implemented with a program such as POLARRFN (“The CCP4 suite: programs for protein crystallography”, Acta Crystallogr. D, 50:760-763, (1994)) to deduce the presence of symmetry elements such as a noncrystallographic dyad. When an initially-derived model is found to be both inaccurate and incomplete (for example, accounting for only ˜45% of the total scattering matter in the asymmetric unit), an aggressive density modification protocol can be undertaken. Such a protocol can comprise iterative cycles of two-fold NCS averaging in DM (CCP4, 1994), interspersed with model building in MOLOC (Muller, et al., Bull. Soc. Chim. Belg., 97:655-667, (1988)), and model refinement in REFMAC (Murshudov, et al., Acta Crystallogr. D, 53:240-255, (1997)). Other procedures include MAMA (Kleywegt, et al., “Halloween . . . masks and bones,” in From First Map to Final Model, Bailey, et al., eds., Warrington, England, SERC Daresbury Laboratory, 1994) for mask manipulations, and PHASES (Furey, et al., PA33 Am. Cryst. Assoc. Mtg. Abstr. 18:73 (1990)) and the CCP4 suite (CCP4, 1994) for the generation of structure factors and the calculation of weights.

In situations where refinement is hampered by severe model bias, the program DMMULTI (CCP4, 1994) can be used to project averaged density from the complex cell into the cell of a related complex, in order to reduce model bias. Using MOLOC, a model of the LBD was built into the resulting density. Models may also be refined with the simulated annealing, positional and B-factor refinement protocols in X-PLOR (Brunger, X-PLOR Version 3.843, New Haven, Conn.: Yale University, 1996) using a maximum-likelihood target (Adams, et al., Proc. Natl. Acad. Sci. USA, 94:5018-23, (1997)). Anisotropic scaling and a bulk solvent correction can be used and all B-factors are preferably refined isotropically. The program PROCHECK (CCP4, 1994) can be used to check whether the residues in the model are in the core regions of the Ramachandran plot or whether any are in the disallowed regions.

Since nuclear receptor LBD's may crystallize in more than one crystal form, the structural coordinates of AR, its LBD, or portions thereof, as provided in tables 1 and 2, in the files identified respectively as Table1_ARLBD_DHT_CDP.txt and Table2_ARLBD_DHT_CRP.txt, presented on CD-R herewith, are particularly useful to solve the structure of those other crystal forms of nuclear receptors. The structural coordinates may also be used to solve the structure of mutants or co-complexes of other nuclear receptors that have sufficient homology.

One method that may be employed for solving other crystal structures is molecular replacement. In this method, the unknown crystal structure may be determined using the structural coordinates of the present invention, as provided in Tables 1 and 2, in the files identified respectively as Table1_ARLBD_DHT_CDP.txt and Table2_ARLBD_DHT_CRP.txt, presented on CD-R herewith. This method will provide an accurate structural form for the unknown crystal more quickly and efficiently than attempting to determine such information ab initio.

Structural Coordinates of AR Bound with Coactivators

The present invention provides, for the first time, the high-resolution three-dimensional structures and atomic structure coordinates of AR bound with a coactivator. The specific methods used to obtain the structure coordinates are provided in the examples, herein. The atomic structure coordinates of AR bound with a coactivator, are listed in Tables 1 and 2, in the files identified respectively as Table1_ARLBD_DHT_CDP.txt and Table2_ARLBD_DHT_CRP.txt, presented on CD-R herewith.

Structure coordinates for AR according to Appendix 1 may be modified by mathematical manipulation. Such manipulations include, but are not limited to, fractionalization of the raw structure coordinates, additions to, or subtractions from, sets of the raw structure coordinates, by a constant amount inversion, rotation, or reflection the raw structure coordinates, and any combination of the foregoing.

Those having ordinary skill in the art will recognize that atomic structure coordinates are not without error. Thus, it is to be understood that, preferably, any set of structure coordinates obtained for AR, that have a root mean square deviation (“r.m.s.d.”) of from about 0.5 to about 0.7 Å, or from 0.5 to 0.7 Å, when superimposed, using backbone atoms (N, Cα, C and O), on the structure coordinates listed in any one of the structures whose coordinates are found in Tables 1 and 2, in the files identified respectively as Table1_ARLBD_DHT_CDP.txt and Table2_ARLBD_DHT_CRP.txt, presented on CD-R herewith, are considered to be identical with the structure coordinates listed herein when at least about 50% to 100% of the backbone atoms of AR are included in the superposition. Less preferably, a set of structure coordinates obtained for AR that have a r.m.s.d. of from about 0.7 to about 1.0 Å, or from 0.7 to 1.0 Å, when superimposed, as previously described, can be considered to be identical with the structure coordinates listed herein.

As used herein, the term “portion thereof” when referring to the LBD, or a coactivator binding site, is intended to mean the atomic coordinates corresponding to a sufficient number of residues or their atoms that interaction with a compound capable of binding to the site can be accurately described. This includes receptor residues having an atom within about 4.5 Å of a bound compound or fragment thereof. Thus, for example, the atomic coordinates provided to a computer modeling system can contain atoms of the nuclear receptor LBD, part of the LBD such as atoms corresponding to the coactivator binding site, or a subset of atoms useful in the modeling and design of compounds that bind to a LBD.

Representations of Structure Coordinates

The atomic structure coordinates of AR bound with a coactivator can be used in molecular modeling and design, as further described hereinbelow. All format representations of the coordinates described herein, or portions thereof, are contemplated by the present invention. Accordingly, the present invention encompasses the structure coordinates and other information, e.g., amino acid sequence, connectivity tables, vector-based representations, temperature factors, etc., used to generate the three-dimensional structure of the coactivator-bound AR for use in the software programs described herein and other software programs.

While Cartesian coordinates are important and convenient representations of the three-dimensional structure of a protein or polypeptide, those of ordinary skill in the art will readily recognize that other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the molecule is chosen, a second atom is placed at a defined distance from the first atom, a third atom is placed at a defined distance from the second atom so that the first, second and third atoms, when taken in order, make a defined angle. Each subsequent atom is placed at a defined distance from a previously placed atom to make a specified angle with respect to a third atom, and at a specified torsion angle with respect to a fourth atom.

Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the molecule structure. Furthermore, the positions of atoms in a three-dimensional structure may be represented as fractions of the unit cell (fractional coordinates), or in spherical polar coordinates.

Additional information, such as thermal parameters, which measure the motion of each atom in a crystal structure, chain identifiers, which identify the particular chain of a multi-chain protein in which an atom is located, and connectivity information, which indicates to which atoms a particular atom is bonded, are also useful for representing a three-dimensional molecular structure.

A variety of data processor programs and formats can be used to store the sequence and structure information on a computer readable medium. Such formats include, but are not limited to, Protein Data Bank (“PDB”) format (Research Collaboratory for Structural Bioinformatics; http://www.rcsb.org/pdb/docs/format/pdbguide2.2/guide2.2_frame.html); Cambridge Crystallographic Data Centre format (see www.ccdc.cam.ac.uk/support/csd_doc/volume3/z323.html); Structure-data (“SD”) file format (MDL Information Systems, Inc.; Dalby et al., J. Chem. Inf. Comp. Sci. 32:244-255, (1992)), and line-notation, e.g., as used in SMILES (Weininger, D., “SMILES, a Chemical Language and Information System. 1. Introduction to Methodology and Encoding Rules,” J. Chem. Inf. Comp. Sci., 28:31-36, (1988)), and CHUCKLES (Siani, M. A., Weininger, D., Blaney, J., “CHUCKLES: a method for representing and searching peptide and peptoid sequences on both monomer and atomic levels,” J. Chem. Inf. Comp. Sci., 34:588-593, (1994)).

Methods of converting between various formats read by different computer software will be readily apparent to those of ordinary skill in the art, and programs for carrying out such conversions are widely available, either as stand-alone programs, e.g., BABEL (v. 1.06, Walters, P. & Stahl, M., © 1992, 1993, 1994; http://smog.com/chem/babe1/ and http://www.brunel.ac.uk/departments/chem/babe1.htm) or integrated into other software packages.

Subsets of the atomic structure coordinates of the present invention can be used in any of the methods described herein. Particularly useful subsets of the coordinates include, but are not limited to, coordinates of single domains of AR, in particular the LBD, coordinates of residues lining an active site such as the coactivator binding site, coordinates of residues that participate in important intramolecular, or intermolecular, contacts at an interface, and Ca coordinates. For example, the coordinates of one domain of a protein that contains the active site may be used to design inhibitors that bind to that site, even though the protein is fully described by a larger set of atomic coordinates. Therefore, a set of atomic coordinates that define the entire polypeptide chain of AR, or the AR ligand binding domain, although useful for many applications, do not necessarily need to be used for the methods described herein.

Data Storage Media

After the three dimensional structure of a cocrystal of AR with a coactivator is determined, the structural information, comprising atomic coordinates obtained from the crystals of the present invention, can be stored electronically. Accordingly, the present invention encompasses machine readable media embedded with the three-dimensional structure of the model described herein, or with portions thereof and/or X-ray diffraction data. By providing a computer readable medium having stored thereon the atomic coordinates of the invention, one of ordinary skill in the art can routinely access the atomic coordinates of the invention, or portions thereof, and related information for use in modeling and design programs, as described in detail hereinbelow.

As used herein, “machine readable medium” or “computer readable medium” refers to any media that can be read and accessed directly by a computer or scanner. Such media include, but are not limited to: magnetic storage media, such as floppy discs, hard discs and magnetic tape; optical storage media such as optical discs; CD-ROM, CD-R or CD-RW, and DVD; electronic storage media such as RAM or ROM; and hybrids of these categories such as magnetic/optical storage media. In a preferred embodiment, the information is provided in the form of a machine-readable data storage medium such as a CD-Rom, or on a computer hard-drive. Such media further include paper on which is recorded a representation of the atomic structure coordinates, e.g., Cartesian coordinates, that can be read by a scanning device and converted into a three-dimensional structure with optical character recognition (OCR) technology. The choice of the data storage structure will generally be based on the means chosen to access the stored information.

The data storage medium preferably also contains information for constructing and/or manipulating an atomic model of a nuclear receptor ligand binding domain, such as the AR LBD and the coactivator binding site, or portion thereof.

The atomic coordinates preferably comprise the coordinates of amino acids in the LBD and coactivator binding site that are responsible for key interactions between the AR and an androgen and a coactivator, respectively. For example, the machine readable data for the ligand binding domain preferably comprises structure coordinates of amino acids corresponding to human AR residues of N-terminal helix 3 (Leu 712, Val 713, and Val716), helix 4 (Pro 723 and Phe725), helix 5 (Gln 733, Met734, Ile737, and Gln738), helix 6 (Trp741), and C-terminal helix 12 (Glu 893, Met894, Glu 897 and Ile898), or a homologue of the molecule or molecular complex comprising the coactivator binding site. The homologues comprise a LBD that has a root mean square deviation from the backbone atoms of the amino acids preferably of not more than 2.0 Å, more preferably 1.8 Å, and still more preferably 1.5 Å.

Subsets of the atomic structure coordinates can be used in any of the methods described herein. Particularly useful subsets of the coordinates include, but are not limited to, coordinates of single domains, coordinates of residues lining an active site, coordinates of residues that participate in important protein-protein contacts at an interface, and Cα coordinates. For example, the coordinates of one domain of a protein that contains the active site may be used to design inhibitors that bind to that site, even though the protein is fully described by a larger set of atomic coordinates. Therefore, a set of atomic coordinates that define the entire polypeptide chain, although useful for many applications, do not necessarily need to be used for the methods described herein.

The machine-readable data storage medium can be used for molecular replacement studies utilizing methods familiar to one of ordinary skill in the art. For example, a data storage material is encoded with a first set of machine-readable data that can be combined with a second set of machine-readable data. For molecular replacement, the first set of data can comprise a Fourier transform of at least a portion of the structural coordinates of the nuclear receptor or portion thereof of interest, and the second data set comprises an X-ray diffraction pattern of the molecule or molecular complex of interest. Using a machine programmed with instructions for using the first and second data sets, a portion or all of the structure coordinates corresponding to the second data can be determined.

As is understood by one of ordinary skill in the art, where structure coordinates have previously been determined and made available to the public, they may be obtained from a source such as the Protein Data Bank (PDB, see for example, www.rcsb.org/pdb). In the alternative, where a closely similar structure is known or available, the structure of interest can be built up using principles of homology modeling. Programs, often embedded within a larger molecular modelling package or suite of related programs, are available to one of ordinary skill in the art for the purpose of homology modelling. For examples of homology modelling tools, see: SEGMOD, part of LOOK (Levitt, (1992), J. Mol. Biol. 226: 507-533; Levitt, (1983), J. Mol. Biol. 170: 723-764; formerly available from the Molecular Applications Group, Palo Alto, Calif.); The Structure Prediction tool within the Molecular Operating Environment (MoE), (Chemical Computing Group Inc., 1010 Sherbrooke Street West, Suite 910, Montreal, Quebec, Canada, see www.chemcomp.com/article/homology.htm); Modeler (within the Quanta suite of programs, available from Accelrys, a subsidiary of Pharmacopeia, Inc.; see also www.accelrys.com/quanta/modeler.html#ahm); and COMPOSER (Blundell et al., see e.g., Protein Eng., 1:377-384, (1987); available as part of the Sybyl package, from Tripos, Inc., 1699 South Hanley Road, St. Louis, Mo.; see www.tripos.com/sciTech/in SilicoDisc/bioInformatics/composer.html).

The machine readable data storage medium can also be used in computational methods of interactive drug design, specifically the design of synthetic molecules that bind to the LBD of AR, and to the coactivator binding site of AR, as well as other related nuclear receptors.

In one embodiment of the present invention, the structure coordinates of the ligand binding domain and coactivator binding site of AR are useful for identifying and/or designing compounds that bind AR so that new therapeutic agents may ultimately be developed.

Under certain conditions, a high resolution X-ray structure can be obtained that shows the locations of ordered solvent molecules around the protein, and in particular at or near putative binding sites on the protein. This information can then be used to design molecules that bind these sites, the compounds synthesized and tested for binding in biological assays. See, for example, Travis, “Proteins and Organic Solvents Make an Eye-Opening Mix”, Science, 262:1374, (1993).

In another embodiment, the structure of the AR coactivator binding site is probed with a plurality of molecules to determine their ability to bind to AR at various sites. Such compounds can be used as targets or leads in medicinal chemistry efforts to identify modulators, for example, inhibitors of potential therapeutic importance.

Structure-activity relationships can further be determined through routine testing using the assays described herein and otherwise known in the art.

computational Methods and Computer Systems

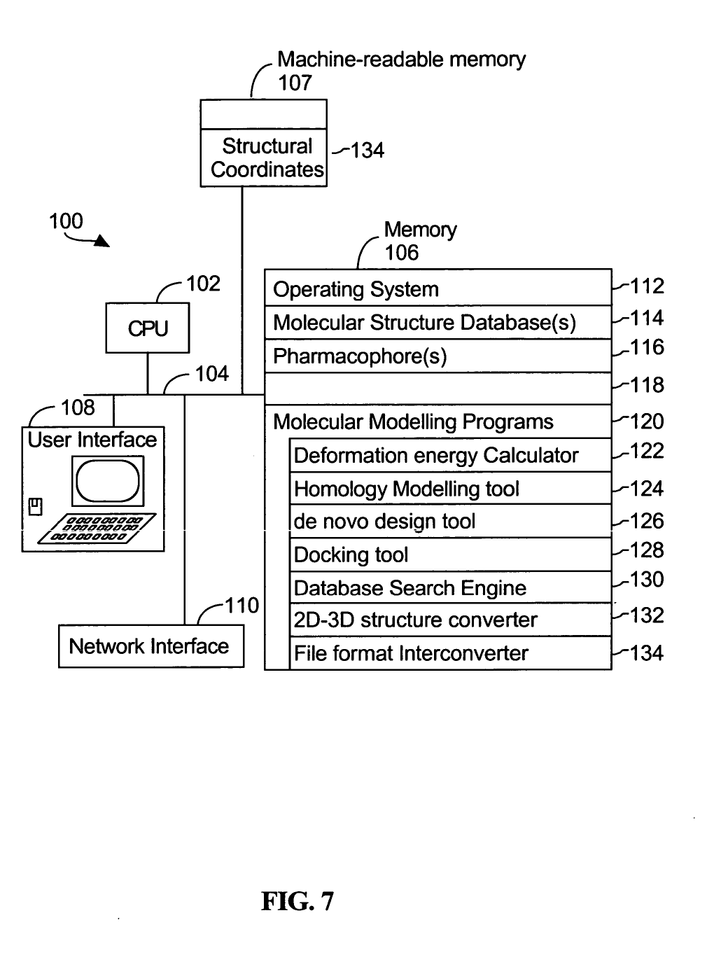

The structural coordinates of the proteins of the present invention are preferably stored in electronic form on a computer-readable medium for use with a computer. Additionally, methods of rational drug design and virtual screening that utilize the coordinates of the proteins of the present invention are preferably performed on one or more computers, as depicted in FIG. 7.

According to FIG. 7, a computer system 100 on which methods of the present invention may be carried out, comprises: at least one central-processing unit 102 for processing machine readable data, coupled via a bus 104 to working memory 106, a user interface 108, a network interface 110, and a machine-readable memory 107.

Machine-readable memory 107 comprises a data storage material encoded with machine-readable data, wherein the data comprises the structural coordinates 134 of at least one cocrystal of AR, or its ligand binding domain, with a ligand and a coactivator; and

Working memory 106 stores an operating system 112, optionally one or more molecular structure databases 114, one or more pharmacophores 116 derived from structural coordinates 134, a graphical user interface 118 and instructions for processing machine-readable data comprising one or more molecular modelling programs 120 such as a deformation energy calculator 122, a homology modelling tool 124, a de novo design tool, 126, a “docking tool” 128, a database search engine 130, a 2D-3D structure converter 132 and a file format interconverter 134.

Computer system 100 may be any of the varieties of laptop or desktop personal computer, or workstation, or a networked or mainframe computer or super-computer, that would be available to one of ordinary skill in the art. For example, computer system 100 may be an IBM-compatible personal computer, a Silicon Graphics, Hewlett-Packard, Fujitsu, NEC, Sun or DEC workstation, or may be a supercomputer of the type formerly popular in academic computing environments. Computer system 100 may also support multiple processors as, for example, in a Silicon Graphics “Origin” system.

Operating system 112 may be any suitable variety that runs on any of computer systems 100. For example, in one embodiment, operating system 112 is selected from the UNIX family of operating systems, for example, Ultrix from DEC, AIX from IBM, or IRIX from Silicon Graphics. It may also be a LINUX operating system. In another embodiment, operating system 112 may be a VAX VMS system. In a preferred embodiment, operating system 112 is a Windows operating system such as Windows 3.1, Windows NT, Windows 95, Windows 98, Windows 2000, or Windows XP. In yet another embodiment, operating system 112 is a Macintosh operating system such as MacOS 7.5.x, MacOS 8.0, MacOS 8.1, MacOS 8.5. MacOS 8.6, MacOS 9.x and MaxOS X.

The graphical user interface (“GUI”) 118 is preferably used for displaying representations of structural coordinates 134, or variations thereof, in 3-dimensional form on user interface 108. GUI 118 also preferably permits the user to manipulate the display of the structure that corresponds to structural coordinates 134 in a number of ways, including, but not limited to: rotations in any of three orthogonal degrees of freedom; translations; projecting the structure on to a 2-dimensional representation; zooming in on specific portions of the structure; coloring of the structure according to a property that varies amongst to different regions of the structure; displaying subsets of the atoms in the structure; coloring the structure by atom type; displaying tertiary structure such as α-helices and β-sheets as solid or shaded objects; and displaying a surface of a small molecule, peptide, or protein, as might correspond to, for example, a solvent accessible surface, also optionally colored according to some property. Structural coordinates 134 are also optionally copied into memory 106 to facilitate manipulations with one or more of the molecular modelling programs 120.

Network interface 110 may optionally be used to access one or more molecular structure databases stored in the memory of one or more other computers.

The computational methods of the present invention may be carried out with commercially available programs which run on, or with computer programs that are developed specially for the purpose and implemented on, computer system 100. Commercially available programs typically comprise large integrated molecular modelling packages that contain at least two of the types of molecular modelling progams 120 shown in FIG. 7. Examples of such large integrated packages that are known to those skilled in the art include: Cerius2 (available from Accelrys, a subsidiary of Pharmacopeia, Inc.; see also www.accelrys.com/cerius2/index.html), Molecular Operating Environment (available from, Chemical Computing Group Inc., 1010 Sherbrooke Street West, Suite 910, Montreal, Quebec, Canada; see www.chemcomp.com/fdept/prodinfo.htm), Sybyl (available from Tripos, Inc., 1699 South Hanley Road, St. Louis, Mo.; see www.tripos.com/software/sybyl.html) and Quanta (available from Accelrys, a subsidiary of Pharmacopeia, Inc.; see also www.accelrys.com/quanta/index.html).

Alternatively, the computational methods of the present invention may be performed with one or more stand-alone programs each of which carries out one of the functions performed by molecular modelling progams 120. In particular, certain aspects of the display and visualization of molecular structures may be accomplished by specialized tools, for example, GRASP (Nicholls, A.; Sharp, K.; and Honig, B., PROTEINS, Structure, Function and Genetics, (1991), Vol. 11 (No. 4), 281; available from Dept. Biochem., Room 221, Columbia University, Box 36, 630 W. 168th St., New York, N.Y.; see also trantor.bioc.columbia.edu/grasp/).

Molecular Modelling Methods In General

Structure information, typically in the form of the atomic structure coordinates, can be used in a variety of computational or computer-based methods to, for example, design, screen for and/or identify compounds that bind the ligand binding domain of AR or the coactivator binding site of AR, or to intelligently design mutants of AR that have altered biological properties with respect to hormones and coactivators.

In another embodiment, compounds that can isomerize to short-lived reaction intermediates in the chemical reaction of an AR-binding compound with AR can be developed. Thus, the analysis of time-dependent structural changes in AR during its interaction with other molecules such as hormones and coactivators is within the scope of the present invention. The reaction intermediates of AR can also be deduced from the reaction product in co-complex with AR. Such information is useful to design improved analogues of known AR modulators, e.g., inhibitors, or to design novel classes of modulators based on the reaction intermediates of AR-inhibitor co-complexes. This provides a novel route for designing AR modulators, e.g., inhibitors, with both high specificity and stability.

In still another embodiment, the structure of the AR ligand binding domain an coactivator binding site can be used to computationally screen small molecule databases for functional groups or compounds that can bind in whole, or in part, to AR. In this screening, the quality of fit of such entities or compounds to the binding site may be judged by methods such as shape complementarity or by estimated interaction energy. See, for example, Meng et al., (1992), J. Comp. Chem., 13:505-524.

Compounds fitting the coactivator binding site serve as a starting point for an iterative design, synthesis and test cycle in which new compounds are selected and optimized for desired properties including affinity, efficacy, and selectivity with respect to the AR coactivator binding site and various mutants thereof. For example, the compounds can be subjected to additional modification, such as replacement and/or addition of R-group substituents of a core structure identified for a particular class of binding compounds, modeling and/or activity screening if desired, and then subjected to additional rounds of testing.

By “modeling” is intended to mean quantitative and qualitative analysis of molecular structure and/or function based on atomic structural information and interaction models of a receptor and a ligand agonist or antagonist. Modeling thus includes conventional numeric-based molecular dynamic and energy minimization models, interactive computer graphic models, modified molecular mechanics models, distance geometry and other structure-based constraint models. Modeling is preferably performed using a computer and may be further optimized using methods familiar to one of ordinary skill in the art.

Docking

Identification of the coactivator binding site structure has made it possible to apply the principles of molecular recognition to design a compound which is complementary to the structure of the site. Accordingly, computer programs that employ various docking algorithms can be used to identify compounds that fit into the ligand binding domain of AR and its coactivator binding site. Such information can be used to predict how a molecule of interest would interact with the coactivator binding site of another nuclear receptor. Fragment-based docking can also be used to build molecules de novo inside the coactivator binding site, by placing molecular fragments that have a complementary fit with the site, thereby optimizing intermolecular interactions. Techniques of computational chemistry can further be used to optimize the geometry of the bound conformations.

Docking may be accomplished using commercially available software such as QUANTA (available from Accelrys, a subsidiary of Pharmacopeia, Inc.; see also www.accelrys.com/quanta/index.html); SYBYL, (available from Tripos, Inc., 1699 South Hanley Road, St. Louis, Mo.; see www.tripos.com/software/sybyl.html), DOCK (Kuntz et al., (1982), J. Mol. Biol., 161:269-288, available from University of California, San Francisco, Calif., see dock.compbio.ucsf.edu/dockinfo.html); GOLD (Jones, et al., (1995), J. Mol. Biol., 245:43-53, available from the Cambridge Crystallographic Data Centre, 12 Union Road. Cambridge, U.K.; see www.ccdc.cam.ac.uk/prods/gold/index.html); AUTODOCK (Goodsell & Olsen, (1990), Proteins: Structure, Function, and Genetics 8:195-202, available from Scripps Research Institute, La Jolla, Calif., see also www.scripps.edu/pub/olson-web/doc/autodock/); GLIDE (available from Schrodinger, Inc., Portland, Oreg., see www.schrodinger.com/Products/glide.html); and ICM (Abagayan, et al., see http://www.molsoft.com/products/modules/dock.htm, available from MolSoft, L.L.C., 3366 North Torrey Pines Court, Suite 300, La Jolla, Calif.).

Docking is typically followed by energy minimization and molecular dynamics simulations of the docked molecule, using molecular mechanics forcefields such as MM2 (see, e.g., Rev. Comp. Chem., 3, 81 (1991)), MM3 (Allinger, N. L., Bowen, J. P., and coworkers, University of Georgia; see, J. Comp. Chem., 17:429 (1996); available from Tripos, Inc., 1699 South Hanley Road, St. Louis, Mo.; see www.tripos.com/software/mm3.html), CHARMM (see, e.g., B. R. Brooks, R. E. Bruccoleri, B. D. Olafson, D. J. States, S. Swaminathan, and M. Karplus, “CHARMM: A Program for Macromolecular Energy, Minimization, and Dynamics Calculations,” J. Comp. Chem., 4, 187-217, (1983)), a version of AMBER such as version 7, (Kollman, P. A., et al., School of Pharmacy, Department of Pharmaceutical Chemistry, University of California at San Francisco, see http://amber.scripps.edu/), and Discover (available from Accelrys, a subsidiary of Pharmacopeia, Inc.; see also www.accelrys.com/insight/discover.html).

Constructing Potential Molecules That Bind to AR

A compound that binds to AR, thereby exerting a modulatory or other effect on its function, may be computationally designed and evaluated by means of a series of steps in which functional groups or other fragments are screened and selected for their ability to associate with the individual binding pockets or other areas of AR. One of ordinary skill in the art may use one of several methods to screen functional groups and fragments for their ability to associate with AR. This process may begin by visual inspection of, for example, the coactivator binding site on the computer display based on the coordinates of AR. Selected fragments or functional groups may then be positioned in a variety of orientations, or docked, within an individual binding pocket of AR as described hereinabove.

Specialized computer programs may assist in the process of selecting fragments or functional groups, or whole molecules that can populate a binding site, or can be used to build virtual combinatorial libaries. These include: GRID (Goodford, (1985), J. Med. Chem., 28:849-857). GRID is available from Oxford University, Oxford, UK; and MCSS (Miranker & Karplus, (1991), Proteins: Structure, Function and Genetics 11:29-34). MCSS is available from Accelrys, a subsidiary of Pharmacopeia, Inc., as part of the Quanta package; see also http://www.accelrys.com/quanta/mcss_hook.html.

Once suitable functional groups or fragments have been selected, they can be assembled into a single compound or inhibitor. Assembly may proceed by visual inspection of the relationship of the fragments to each other in relation to a three-dimensional image of the structure coordinates of the ligand binding domain, and coactivator binding site of AR displayed on a computer display. This would typically be followed by manual model building using software such as QUANTA or SYBYL.

Alternatively, and preferably, useful programs to aid one of skill in the art in connecting the individual functional groups or fragments include: CAVEAT (Bartlett et al., “CAVEAT: A Program to Facilitate the Structure-Derived Design of Biologically Active Molecules,” in Molecular Recognition in Chemical and Biological Problems, Speciai Pub., Royal Chem. Soc. 78:182-196, (1989). CAVEAT is available from the University of California, Berkeley, Calif.); 3D Database systems such as MACCS-3D (MDL Information Systems, San Leandro, Calif.); and HOOK (available from Accelrys, a subsidiary of Pharmacopeia, Inc., as part of the Quanta package; see also www.accelrys.com/quanta/mcss_hook.html). This area is reviewed in Martin, Y. C., J. Med. Chem., 35:2145-2154, (1992).

Instead of proceeding to build a modulator of AR in a step-wise fashion one fragment or functional group at a time, as described hereinabove, AR binding compounds may be designed as a whole or de novo using either an empty active site or optionally including some portion(s) of a known ligand. Programs for achieving this include: LUDI (Bohm, J. Comp. Aid. Molec. Design, 6:61-78, (1992), available from Accelrys, a subsidiary of Pharmacopeia, Inc., as part of the Insight package www.accelrys.com/insight/ludi.html); LEGEND (Nishibata and Itai, Tetrahedron, 47:8985, (1991), available from Molecular Simulations, Burlington, Mass.); and LeapFrog (available from Tripos, Inc., 1699 South Hanley Road, St. Louis, Mo.; www.tripos.com/custResources/softwareFAQ/sybyl/ligand_tools/leapfrog.html).

Quantifying Potential Binding Molecules

Once a compound has been designed or selected by methods such as those described hereinabove, the efficiency with which that compound may bind to the coactivator binding site of AR may be tested and optimized by computational evaluation. For example, a compound that has been designed or selected to function as an inhibitor (antagonist) of coactivator binding to AR preferably occupies a volume that does not overlap with the volume occupied by the active site residues when the native substrate is bound. An effective inhibitor of coactivator binding to AR preferably demonstrates a relatively small difference in energy between its bound and free states (i.e., it has a small deformation energy of binding). Thus, the most efficient inhibitors of AR coactivator binding should preferably be designed with a deformation energy of binding of not greater than about 10 kcal/mol or, even more preferably, not greater than about 7 kcal/mol. Inhibitors of AR coactivator binding to AR may interact with the receptor in more than one conformation that is similar in overall binding energy. In such cases, the deformation energy of binding is preferably taken to be the difference between the energy of the free compound and the average energy of the conformations observed when the inhibitor binds to the receptor.

A compound selected or designed for binding to AR may be further computationally optimized so that in its bound state it would lack repulsive electrostatic interactions with AR or the AR LBD. Such repulsive electrostatic interactions include non-complementary interactions such as repulsive charge-charge, dipole-dipole and charge-dipole interactions. Specifically, the sum of all electrostatic interactions between the inhibitor and the receptor when the inhibitor is bound to it preferably make a neutral or favorable contribution to the enthalpy of binding.

Specific computer software is available in the art to evaluate compound deformation energy and electrostatic interaction. Examples of programs designed for such uses fall into approximately three levels of sophistication. The crudest level of approximation, molecular mechanics, is also the cheapest to compute and can most usefully be used to calculate deformation energies. Molecular mechanics programs find application for calculations on small organic molecules as well as polypeptides, nucleic acids, proteins, and most other biomolecules. Examples of programs which have implemented molecular mechanics force fields include: AMBER, such as version 7 (Kollman, P. A., et al., School of Pharmacy, Department of Pharmaceutical Chemistry, University of California at San Francisco, see http://amber.scripps.edu/); CHARMM (see B. R. Brooks, R. E. Bruccoleri, B. D. Olafson, D. J. States, S. Swaminathan, and M. Karplus, “CHARMM: A Program for Macromolecular Energy, Minimization, and Dynamics Calculations,” J. Comp. Chem., 4, 187-217, (1983); A. D. MacKerell, Jr., B. Brooks, C. L. Brooks, III, L. Nilsson, B. Roux, Y. Won, and M. Karplus, “CHARMM: The Energy Function and Its Parameterization with an Overview of the Program,” in The Encyclopedia of Computational Chemistry, 1, 271-277, P. v. R. Schleyer et al., eds, John Wiley & Sons, Chichester, (1998); and see yuri.harvard.edu/); QUANTA/CHARMm (available from Accelrys, a subsidiary of Pharmacopeia, Inc.; see also www.accelrys.com/quanta/index.html#charmm); and Insight II/Discover (available from Accelrys, a subsidiary of Pharmacopeia, Inc.; see also www.accelrys.com/insight/index.html).

An intermediate level of sophistication comprises the so-called “semi-empirical” methods, which are relatively inexpensive to compute and are most frequently employed for calculating deformation energies of organic molecules. Examples of program packages that provide semi-empirical capability are MOPAC 2000 (Stewart, J. J. P., et al., available from Schrödinger, Inc., 1500 S.W. First Avenue, Suite 1180, Portland, Oreg.; see www.schrodinger.com/Products/mopac.html) and AMPAC (Holder, A., et al., available from Tripos, Inc., 1699 South Hanley Road, St. Louis, Mo.; see www.tripos.com/sciTech/in SilicoDisc/moleculeModeling/ampac.html).

The highest level of sophistication is achieved by those programs that employ so-called ab initio quantum chemical methods and methods of density functional theory, for example: Gaussian 03, (available from Gaussian, Inc., Carnegie Office Park, Building 6, Suite 230. Carnegie, Pa., see www.gaussian.com/gaussian.com/g03.htm); and Q-Chem2.0 (“A high-performance ab initio electronic structure program,” J. Kong, et al., J. Comput. Chem., 21, 1532-1548, (2000); available from Four Triangle Lane, Suite 160, Export, Pa.; see also www.q-chem.com/). These programs may be installed, for instance, on a computer workstation, as is well-known in the art. Other hardware systems and software packages will be known to those skilled in the art.

Virtual Screening

In general, databases of small molecules can be computationally screened to identify molecules that are likely to bind in whole, or in part, to a nuclear receptor ligand binding domain, or coactivator binding site, of interest. In such screening, the quality of fit of molecules to the binding site in question may be judged by any of a number of methods that are familiar to one of ordinary skill in the art, including shape complementarity (see, e.g., DesJalais, et al., J. Med. Chem., 31:722-729, (1988)) or by estimated interaction energy (Meng, et al., J. Comp. Chem., 13:505-524, (1992)). Such methods are preferably applicable to ranking compounds for their ability to modulate coactivator binding to a nuclear receptor.

In a preferred method, potential binding compounds may be obtained by rapid computational screening. Such a screening comprises testing a large number, which may be hundreds, or may preferably be thousands, or more preferably tens of thousands, or even more preferably hundreds of thousands of molecules whose formulae are known and for which at least one conformation can be readily computed.

The databases of small molecules include any virtual or physical database, such as electronic and physical compound library databases. Preferably, the molecules are obtained from one or more molecular structure databases that are available in electronic form, for example, the “Available Chemicals Directory” (“ACD”, available from MDL Information Systems, Inc., 14600 Catalina Street, San Leandro, Calif.; see www.mdli.com); the National Cancer Institute database (NCIDB, see www.nci.nih.gov; also available from MDL Information Systems, Inc., 14600 Catalina Street, San Leandro, Calif.; see www.mdli.com); the “MDL Drug Data Report” (MDDR, available from MDL Information Systems, Inc., 14600 Catalina Street, San Leandro, Calif.; see www.mdli.com); the Comprehensive Medicinal Chemistry Database (CMC, available from MDL Information Systems, Inc., 14600 Catalina Street, San Leandro, Calif.; see www.mdli.com); the Cambridge Structural Database; the Fine Chemical Database (Rusinko, Chem. Des. Auto. News, 8:44-47 (1993)); and any proprietary database of compounds with known medicinal properties, as is found in a large or small pharmaceutical company.

The molecules in such databases for use with the present invention are preferably stored as a connection table, with or without a 2D representation that comprises coordinates in just 2 dimensions, say x and y, for facilitating visualization on a computer display. The molecules are more preferably stored as at least one set of 3D coordinates corresponding to an experimentally derived or computer-generated molecular conformation. If the molecules are only stored as a connection table or a 2D set of coordinates, then it can be necessary to generate a 3D structure for each molecule before proceeding with a computational screen, for example, if the molecules are to be docked into a receptor structure during screening. Programs for converting 2D molecular structures or molecule connection tables to 3D structures include Converter (available from Accelrys, a subsidiary of Pharmacopeia, Inc.; see also www.accelrys.com/insight/sketcher_converter.html#converter) and CONCORD (A. Rusinko III, J. M. Skell, R. Balducci, C. M. McGarity, and R. S. Pearlman, “CONCORD, A Program for the Rapid Generation of High Quality Approximate 3-Dimensional Molecular Structures,” (1988) The University of Texas at Austin and Tripos Associates, available from Tripos, Inc., 1699 South Hanley Road, St. Louis, Mo.; see www.tripos.com/sciTech/in SilicoDisc/chemInfo/concord.html)).

As part of a computational screen, it is possible to “dock” 3D structures of molecules from a database into the coactivator binding site of AR, on a high throughput basis. Such a procedure can normally be subject to a number of user-defined parameters and thresholds according to desired speed of throughput and accuracy of result. Such parameters include the number of different starting positions from which to start a docking simulation and the number of energy calculations to carry out before rejecting or accepting a docked structure. Such parameters and their choices are familiar to one of ordinary skill in the art. Structures from the database can be selected for synthesis to test their ability to modulate nuclear receptor activity if their docked energy is below a certain threshold. Methods of docking are further described elsewhere herein.

Alternatively, it is possible to carry out a “molecular similarity” search for molecules that are potential inhibitors of AR coactivator binding. If a pharmacophore has been developed from a knowledge of the AR coactivator binding site, then molecules whose structures map on to that pharmacophore are to be found. A pharmacophore defines a set of contact sites on the surface of the coactivator binding site, accompanied by the distances between them. A similarity search attempts to find molecules in a database that have at least one favorable 3D conformation whose structure overlap favorably with the pharmacophore. For example, a pharmacophore may comprise a lipophilic pocket at a particular position, a hydrogen-bond acceptor site at another position and a hydrogen bond donor site at yet another specified position accompanied by distance ranges between them. A molecule that could potentially fit into the active site is one that can adopt a conformation in which a H-bond donor in the active site can reach the H-bond acceptor site on the pharmacophore, a H-bond acceptor in the active site can simultaneously reach the H-bond donor site of the pharmacophore and, for example, a group such as a phenyl ring can orient itself into the lipophilic pocket.

Even where a pharmacophore has not been developed, molecular similarity principles may be employed in a database searching regime (see, for example, Johnson, M. A.; Maggiora, G. M., Eds. Concepts and Applications of Molecular Similarity, New York: John Wiley & Sons (1990)) if at least one molecule that fits well into the coactivator binding site is known. For example, for use with the present invention, one such molecule could consist of residues that form an NR-box binding motif, or the FXXLF motif in ARA70. In a preferred embodiment, it is possible to search for molecules that have certain properties in common with those of the molecule(s) known to bind. For example, such properties include numbers of hydrogen bond donors or numbers of hydrogen bond acceptors, or overall hydrophobicity within a particular range of values. Alternatively, even where a pharmacophore is not known, similar molecules may be selected on the basis of optimizing an overlap criterion with the molecule of interest. For example, where the structures of test molecules that bind are known, a model of the test molecule may be superimposed over the model of the AR coactivator structure of the invention. Numerous methods are known in the art for performing this step, any of which may be used. See, for example, Farmer, Drug Design, 10:119-143, Ariens, ed., Academic Press, New York (1980); U.S. Pat. No. 5,331,573; U.S. Pat. No. 5,500,807; Verlinde, Structure, 2:577-587 (1994); and Kuntz, et al., Science, 257:1078-1082 (1992).

In searching a molecular structure database, a specialized database searching tool that permits searching molecular structures and sub-structures is typically employed. Examples of suitable database searching tools, known to one of ordinary skill in the art are: ISIS/Host and ISIS/Base (available from MDL Information Systems, Inc., 14600 Catalina Street, San Leandro, Calif.; see www.mdli.com), Unity (available from Tripos, Inc., 1699 South Hanley Road, St. Louis, Mo.; www.tripos.com/sciTech/in SilicoDisc/chemInfo/unity.html) or Catalyst (available from Accelrys, a subsidiary of Pharmacopeia, Inc.; see also www.accelrys.com/catalyst/index.html).

Rational Design Considerations

Molecules that bind to the AR coactivator binding site can be designed by a number of methods, including: exploiting available structural and functional information; by deriving a quantitative structure-activity relationship (QSAR); and by using a combination of such information to design new compound libraries. In particular, focused libraries having molecular diversity at one or more particular groups attached to a core structure or scaffold, may be used. Preferably, structural data is incorporated into the iterative design process. For example, one of ordinary skill in the art may use one of several methods to screen molecules or fragments for their ability to associate with the coactivator binding site of a nuclear receptor of interest. This process may begin with visual inspection of, for example, the AR coactivator binding site on a computer screen. Selected fragments or chemical entities may then be positioned into the site, or a portion thereof. Docking may be accomplished using computer software such as Quanta or Sybyl, as described hereinabove, followed by energy minimization and molecular dynamics with standard molecular mechanics force-fields, such as CHARMM and AMBER, as also described hereinabove.

The design of molecules that inhibit coactivator binding to AR according to the present invention generally involves consideration of two factors. The molecule must be capable of first physically, and second structurally, associating with AR. The physical interactions underpinning this association can be covalent or non-covalent. For example, covalent interactions may be important for designing irreversible or “suicide” inhibitors of a protein. Non-covalent molecular interactions that are important in the association of AR with molecules that bind to it include hydrogen bonding, ionic, van der Waals, and hydrophobic interactions. Structurally, the compound must be able to assume a conformation that allows it to associate with the coactivator binding site of AR. Although certain portions of the compound will not directly participate in this association with AR, those portions may still influence the overall conformation of the molecule. This, in turn, may have a significant impact on potency. Such conformational requirements include the overall three-dimensional structure and orientation of a functional group or moleculein relation to all or a portion of the binding site, or the spacing between functional groups of a compound comprising several functional groups that directly interact with AR.

In general, the potential modulatory or binding effect of a compound on AR may be analyzed prior to its actual synthesis and testing by the use of computer modeling techniques. If the theoretical structure of the given compound suggests insufficient interaction and association between it and the AR coactivator binding site, synthesis and testing of the compound need not be carried out. However, if computer modeling indicates a strong interaction, the molecule may then be synthesized and tested for its ability to bind to the AR coactivator binding site and thereby inhibit its activity. In this manner, synthesis of ineffective compounds may be avoided.

Among the computational techniques that enable the rational design of molecules that bind to AR, it is key to have access to visualization tools, programs for calculating properties of molecules, and programs for fitting ligand structures into three-dimensional representations of the receptor binding site. Computer program packages for facilitating each of these capabilities have been referred to herein, and are available to one of ordinary skill in the art. Visualization of molecular properties, such as field properties that vary through space, can also be particularly important and may be aided by computer programs such as MOLCAD (Brickmann, J., and coworkers, see, for example, J. Comp.-Aid. Molec. Des., 7:503, (1993); available from Tripos, Inc., 1699 South Hanley Road, St. Louis, Mo.; www.tripos.com/sciTech/in SilicoDisc/moleculeModeling/molcad.html).

A molecular property of particular interest when assessing suitability of drug compounds is its hydrophobicity. An accepted and widespread measure of hydrophobicity is LogP, the Log₁₀ of the octanol-water partition coefficient. It is customary to use the value of LogP for a designed molecule to assess whether the molecule could be suitable for transport across a cell membrane, if it were to be administered as a drug. Measured values of LogP are available for many compounds. Methods and programs for calculating LogP are also available, and are particularly useful for molecules that have not been synthesized or for which no experimental value of LogP is available. See for example: CLOGP (Hansch, C., and Leo, A.; available from Biobyte, Inc., Pomona, Calif., see also, www.biobyte.com/bb/prod/clogp40.html); and ACD/LogP DB (Advanced Chemistry Development Inc., 90 Adelaide Street West, Suite 702, Toronto, Ontario Canada, www.acdlabs.com/products/phys_chem_lab/logp/).

Other molecular modeling techniques may also be employed in accordance with the present invention. See, for example, Cohen, et al., J. Med. Chem., 33:883-894, (1990) and Navia, et al., Current Opinions in Structural Biology, 2:202-210, (1992). The specific model building techniques and computer evaluation systems described herein are not to be construed as a limitation on the present invention.

Using these computer modeling systems a large number of compounds may be quickly and easily examined so that expensive and lengthy biochemical testing is avoided. Moreover, the need for actual synthesis of many compounds can be substantially reduced and/or effectively eliminated.

Further Manipulations of AR Structures and Molecules Binding Thereto

Once an AR-binding compound has been optimally selected or designed, as described hereinabove, substitutions may then be made in some of its atoms or chemical groups in order to improve or modify its binding properties. Generally, initial substitutions are conservative, i.e., the replacement group will have approximately the same size, shape, hydrophobicity, polarity and charge as the original group. For selection of appropriate groups, any of several chemical models can be used, e.g., isolobal or isosteric analogies. Groups known to be bio-isosteres of one another are particularly preferred. One of skill in the art will understand that substitutions known in the art to alter conformation are preferably avoided. Such altered chemical compounds may then be analyzed for efficiency of binding to AR by the same computer methods described hereinabove.

The structure coordinates of AR mutants will also facilitate the identification of related proteins or enzymes analogous to AR in function, structure or both, thereby further leading to novel therapeutic modes for treating or preventing AR mediated diseases.

Compounds of the Present Invention

Without the benefit of access to the structural coordinates of the AR cocrystals of the present invention, it would be considered that a 3-point organic scaffold would be required to bind to the AR coactivator binding site in such a manner that coactivator binding would be inhibited. To achieve such a 3-point attachment, the inhibitor would have 3 groups that mimic the interaction of, respectively, each of 3 leucines on a coactivator binding motif such as LXXLL with the AR coactivator binding site. However, the understanding of AR binding to ARA70 that has been obtained from the present invention leads to the surprising deduction that a molecule with only two points of attachment could be sufficient to inhibit ARA70 binding.

Suitable peptides for binding to the AR coactivator binding site may also be obtained by modifying coactivator-related peptides, whose identification and synthesis has been described herein. According to methods of the present invention, suitable compounds for binding to the AR coactivator binding site may preferably be made by modifying coactivator-derived peptides such as those derived from ARA70. Such peptides preferably comprise a motif such as FXXLF, FXXLW, FXXFF, FXXLY, FXXYF, WXXVW, or WXXLF, accompanied by various flanking residues as found in ARA70. Alternatively, such peptides may also comprise the motif LXXLL.

Modifications to such coactivator-derived peptides preferably involve imposing conformational constraints such as are obtained through cyclization, and may also involve the use of derivatized side-chains of naturally occurring amino acids. Examples of such methods of making such peptides may be found respectively in: Geistlinger, T. R., and Guy, R. K., “An Inhibitor of the Interaction of Thyroid Hormone Receptor β and Glucocorticoid Interacting Protein 1”, J. Am. Chem. Soc., 123:1525-26, (2001); and Geistlinger, T. R., and Guy, R. K., “Novel Selective Inhibitors of the Interaction of Individual Nuclear Hormone Receptors with a Mutually Shared Steroid Receptor Coactivator 2”, J. Am. Chem. Soc., 125:6852-53, (2003), both of which are incorporated herein by reference in their entirety. According to such methods, certain cyclization of peptides creates conformationally constrained peptides that are pre-locked into favorable binding conformations such as those that have α-helical turns and thereby leads to an increased binding affinity than that of an unconstrained peptide. The cyclizations in question typically involve replacing a pair of residues, at i, and i+4, (i.e., separated by 4 residues), by residues D and K, respectively, and then forming a bridge between the pair of substituted residues. Derivatized side chains of residues, preferably of those within an NR-box motif such as LXXLL or FXXLF, can lead to peptides that have selectivity amongst various nuclear receptors. Derivatized side chains include, but are not limited to, those substituted with groups such as F, CF₃, Cl, thiophenyl, cyclohexyl, and others found in Geistlinger and Guy, J. Am. Chem. Soc., 125:6852-53, (2003).

Any of the modifications described hereinabove can also be applied to any other peptide described herein, including for example coactivator-related peptides, in order to increase binding affinity of the resulting modified peptide. The application of such methods of modifying peptides to would be within the capability of one of ordinary skill in the art.

The methods of the present invention also lead to design of small organic molecules that fit in the AR coactivator binding site in such a way that they would inhibit the binding of a natural coactivator, including the N-terminal domain of AR itself, thereto. Preferably such molecules are designed using a 3-dimensional representation of a cocrystal of the AR LBD with a peptide, either coactivator-derived or coactivator-related, bound to the coactivator binding site. Even more preferably the molecules are designed to mimic a peptide having a motif FXXLF, FXXLW, FXXFF, WXXVW, or WXXLF, as positioned in the coactivator binding site of AR. Such motifs have in common a residue, such as F, or W, that has a bulky side-chain containing an aromatic ring, at positions +1 and +5.

Small organic molecules that mimic just the two attachment points, +1 and +5 of coactivator binding motifs, and peptide mimics thereof, are easier to work with than those that might attempt to mimic the binding of a motif such as LXXLL in which the 3 leucines form separate attachment points to the coactivator binding site.

Accordingly, the present invention includes molecules and compounds of formulae (I) and (II):

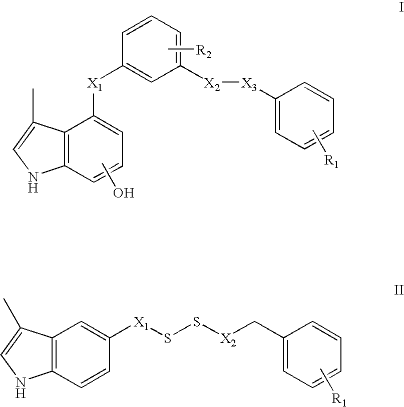

In molecules I and II, R₁ and R₂ may be independently a substituent selected from the group consisting of: hydrogen, alkyl, branched alkyl, alkenyl, branched alkenyl, alkynyl, branched alkynyl, hydroxyl, nitro, sulfoxy, amino, and halide. X₁, X₂, and X₃, may be any linking moiety that is at least bivalent, and which is preferably selected from the group consisting of alkene, alkylene, ether oxy, secondary amine, or a phosphorous containing group. Synthetic routes to molecules I and II are within the capability of one of ordinary skill in the art of synthetic organic chemistry.

In a preferred embodiment, compounds of the present invention bind to a coactivator binding site of the ligand binding domain with greater affinity than the endogenous ligands.

Once a computationally designed ligand (CDL) is synthesized as described herein and known in the art, it can be tested using assays to establish its activity as an agonist, partial agonist or antagonist, and affinity, as described herein. After such testing, the CDL's can be further refined by generating crystals of the AR LBD with a CDL bound to the coactivator binding site. The structure of the CDL can then be further refined using the structural modification methods for three dimensional models described herein to make second generation CDL's with improved activity or affinity. Once a coactivator binding molecule has been optimally selected or designed, as described hereinabove, substitutions may then be made in some of its atoms or functional groups in order to improve or modify its binding properties. Such altered molecules may then be analyzed for efficiency of binding to or modulation of the activity of AR, or a complex thereof, by the methods described in detail hereinabove. Generally, preferred substitutions are conservative, i.e., the replacement group will have approximately the same size, shape, hydrophobicity, polarity and charge as the original group. For selection of appropriate groups, any of several chemical models can be used, e.g., the isolobal analogy, or isosterism. Groups known to be bio-isosteres of one another are particularly preferred. One of ordinary skill in the art will understand that substitutions known in the art to alter conformation should be avoided.

Assays

Compounds identified through modeling can be screened in binding assays such as those familiar to one of ordinary skill in the art in order to identify those that bind strongly to AR, and that may function to inhibit coactivator binding. Such compounds that bind most strongly can be selected to form the basis of drug development programs. Preferred assays for use with the present invention are described in the Examples presented hereinbelow and include, but are not limited to, fluorescence assays, surface plasmon resonance assays, and qualitative assay methods such as “pull-down” assays with appropriate labeling using either fluorescence or radioactive markers. An example of a pull-down assay is a GST-pull-down assay.

Assays, including biological assays, for use with the present invention are characterized by binding of the compound to a ligand binding domain of AR, or some portion thereof such as the coactivator binding site. Screening can be, for example, in vitro, in cell culture, and/or in vivo. Biological screening preferably centers on activity-based response models, binding assays (which measure how well a compound binds to the receptor), and bacterial, yeast and animal cell lines (which measure the biological effect of a compound in a cell). The assays can be automated for high capacity-high throughput screening (HTS) in which large numbers of compounds can be tested to identify compounds with the desired activity. In particular, peptides can be assayed with AR, according to methods described in Peptide library scanning as described in: Chang, C., Norris, J. D., Gron, H., Paige, L. A., Hamilton, P. T., Kenan, D. J., Fowlkes, D., McDonnell, D. P., “Dissection of the LXXLL nuclear receptor-coactivator interaction motif using combinatorial peptide libraries: discovery of peptide antagonists of estrogen receptors alpha and beta”, Mol. Cell. Biol., 19(12):8226-39, (1999)).

As an example of assays that may be used with the methods of the present invention, in vitro binding assays can be performed in which compounds are tested for their ability to block the binding of a ligand, fragment, fusion or peptide thereof, to a nuclear receptor ligand binding domain such as that of AR. For cell and tissue culture assays, the assays may be performed to assess a compound's ability to block the function of cellular coactivators, such as members of the p160 family of coactivator proteins. For example, coactivators include SRC-1, AIB1, RAC3, p/CIP, and GRIP1 and its homologues TIF 2 and NcoA-2, and those that exhibit receptor and/or isoform-specific binding affinity. Tissue profiling and appropriate animal models can also be used to select compounds that bind to the AR coactivator binding site. Different cell types and tissues can also be used for these biological screening assays. Suitable assays for such screening are described in Shibata, et al., Recent Prog. Horm. Res., 52:141-164, (1997); Tagami, et al., Mol. Cell. Biol., 17(5):2642-2648, (1997); Zhu, et al., J. Biol. Chem., 272(14):9048-9054, (1997); Lin, et al., Mol. Cell. Biol., 17(10):6131-6138, (1997); Kakizawa, et al., J. Biol. Chem., 272(38):23799-23804 (1997); and Chang, et al., Proc. Natl. Acad. Sci. USA, 94(17):9040-9045, (1997), all of which are incorporated herein by reference in their entirety.

A preferred assay protocol for use with the present invention is the GST-pulldown assay. GST-pulldown assays to assess peptide inhibition of GST-AR LBD and GRIP-1 interaction can be formatted essentially as described herein. In this embodiment, the interaction of bacterial expressed GST-AR LBD (e.g., in BL21(DE3) cells) with in vitro transcribed and translated ³⁵S-labeled GRIP-1 is monitored by SDS-PAGE analysis of GST-pulldowns. Pre-incubation of GST-AR LBD protein with increasing concentrations of unlabeled competitor peptides (e.g. CRP_{—}1, CRP_{—}3) prior to incubation with ³⁵S-labeled GRIP-1 can be used to determine the relative affinities and efficacies of competitor peptides.

According to a typical protocol for a GST-pulldown assay, total ligand binding activity can be determined by a controlled pore glass bead assay (see, e.g., Greene, et al., Mol. Endocrinol., 2:714-726, (1988)), and protein levels monitored by western blotting with a monoclonal antibody to AR. Cleared extracts containing the GST-LBD's can be incubated in buffer alone (e.g., 50 mM Tris, pH 7.4, 150 mM NaCl, 2 mM EDTA, 1 mM DTT, 0.5% NP-40 and a protease inhibitor cocktail), or with 1 μM of a ligand such as DHT. Incubation times can be about an hour, at 4° C. Extract samples containing sufficient quantity of GST-LBD (e.g., 30 pmol) are incubated with 10 μl glutathione-Sepharose-4B beads (Pharmacia), also for about an hour at 4° C. Beads are washed, preferably about five times, with 20 mM HEPES, pH 7.4, 400 mM NaCl, and 0.05% NP-40.

³⁵S-labeled GRIP1 can be synthesized by in vitro transcription and translation using the TNT Coupled Reticulocyte Lysate System (Promega) according to the manufacturer's instructions and pSG5-GRIP1 as the template. Immobilized GST-LBD's are then incubated for times such as 2.5 hours with 2.5 μl aliquots of crude translation reaction mixture diluted in 300 μl of Tris-buffered saline (TBS). After five washes in TBS containing 0.05% NP-40, proteins can be eluted by boiling the beads for 10 minutes in sample buffer. Bound ³⁵S-GRIP1 can be quantitated by fluorography following SDS-PAGE.

Preferably, binding molecules may be identified by high throughput screening methods, according to which large libraries of ligands are screened against a particular target such as AR. A large library of ligands preferably contains more than 1,000 distinct ligands, more preferably contains more than 10,000 distinct ligands, even more preferably contains more than 100,000 distinct ligands and most preferably contains more than 1,000,000 distinct ligands. High throughput screening methods typically employ robotically controlled assay systems, and take advantage of the latest improvements in miniaturization and automation. Samples are typically assayed on 96-well plates or microtiter plate arrays, and measurements are preferably taken in parallel in order to improve efficiency. For an overview of high throughput screening methods, see, for example, Razvi, E. S., “High Throughput Screening: Where Are We Today?,” Drug &Market Development Publications, (June 1999), and Razvi, E. S., “Industry Trends in High Throughput Screening,” Drug &Market Development Publications, (August 2000).

The compounds selected from assays used with the present invention preferably have antagonist properties with respect to ARA70 binding to AR. The compounds also include those that exhibit previously unknown properties such as varying combinations of agonist and antagonist activities, depending on the effects of altering ligand and/or coactivator binding on the activities of nuclear receptors. Such compounds include, but are not limited to, compounds that have hormone-dependent or hormone-independent activities, compounds which are mediated by proteins other than coactivators, and compounds which interact with the receptors at locations other than the coactivator binding site. The compounds also include those, which through their binding to receptor locations that are conformationally sensitive to hormone binding, have allosteric effects on the receptor by stabilizing or destabilizing the hormone-bound conformation of the receptor, or by directly inducing the same, similar, or different conformational changes induced in the receptor by the binding of hormone.

Methods of Treatment

With the knowledge of coactivator binding obtained by the methods of the present invention, it is of particular interest to design therapeutic compounds that will interact with at least one amino acid residue corresponding to residues of the human androgen receptor selected from the group consisting of: Leu 712, Val 713, Val716, Lys 720, Phe725, Gln 733, Met734, Ile737, Gln738, Trp741, Glu 893, Met894, Glu 897, and Ile898.

Accordingly, one aspect of the present invention is a method of modulating nuclear receptor activity in a mammal by administering to a mammal in need thereof a sufficient amount of a coactivator that fits spatially and preferentially into a coactivator binding site of the androgen receptor, wherein the coactivator is designed by a computational method so that at least one amino acid residue of the androgen receptor coactivator binding site selected from the group consisting of Leu 712, Val 713, Val716, Lys 720, Phe725, Gln 733, Met734, Ile737, Gln738, Trp741, Glu 893, Met894, Glu 897, and Ile898, interacts with at least one functional group of the coactivator. Such a method may involve optimizing the binding capability of the compound by identifying at least one chemical modification of the functional group that produces a second functional group that has a structure that increases an interaction between the interacting amino acid and the second functional group as compared to the interaction between the interacting amino acid and the first functional group.

Compounds designed by this method can be used in conjunction with either agonists or antagonists of AR activity. Thus the method of modulating nuclear receptor activity can comprise administering an antagonist of coactivator binding alone, or an agonist in combination with a coactivator or a compound that mimics a coactivator by binding to the coactivator binding site.

The compounds discovered by methods of the present invention may be used in a method of modulating nuclear receptor activity in a mammal. Specifically, by administering to a mammal in need thereof a sufficient amount of a compound that fits spatially and preferentially into a coactivator binding site of the androgen receptor, it is possible to inhibit androgen receptor coactivator binding in the mammal.

Pre-clinical candidate compounds designed by methods of the present invention can be tested in appropriate animal models in order to measure efficacy, absorption, pharmacokinetics and toxicity using standard techniques known in the art. Compounds exhibiting desired properties can then be tested in clinical trials for use in treatment of various AR-based disorders, such as androgen insensitivity syndrome (AIS), and prostate cancer. Compounds designed by methods of the present invention may also be used to treat other nuclear receptor-based disorders. These include GR-based disorders, including Type II diabetes and inflammatory conditions such as rheumatic diseases.

Tissue-specific Antagonists of Coactivator Binding

The methods of the present invention may be used to develop compounds that selectively inhibit coactivator binding against the androgen receptor in a particular target tissue. Such compounds can be discovered and/or designed by the methods described herein, then screened for tissue specificity by methods that are well known in the art. For example, antagonists of coactivator binding for the androgen receptor in prostate, bone, and muscle tissue may be designed by the methods of the present invention. While the tissue-selective antagonism of coactivators can probably be attributed to numerous factors, dissection of the mechanisms of action of these coactivators is facilitated by a comprehensive understanding of how they act on the AR coactivator binding site and regulate its interactions with other cellular factors.

Coactivators designed by the methods of the instant invention could be used in a suitably designed assay to determine their specificity. Alternatively, the effective levels in a given tissue could be modulated by administering known coactivator inhibitors designed by the methods of the instant invention. The crystal structure of the AR LBD/DHT/GRIP1 peptide complex described herein precisely defines the binding site that would be targeted.

A selective inhibitor of coactivator binding can be designed by a computational method wherein at least one amino acid residue of a nuclear receptor coactivator binding site that corresponds to AR residues Leu 712, Val 713, Val716, Lys 720, Phe725, Gln 733, Met734, Ile737, Gln738, Trp741, Glu 893, Met894, Glu 897, and Ile898, interacts with at least one functional group of the coactivator inhibitor. The method involves overlapping an atomic model of a test molecule with the coordinates of a known coactivator peptide docked into the AR coactivator binding site. The method further comprises identifying a fragment of the test molecule that fits into a cleft in the coactivator binding site that is occupied by the W+1 residue of the peptide. Thus the test molecule preferably interacts with at least one residue selected from the group consisting of: Leu 712, Val 716, Met 734, Gln 738, Met 894, and Ile 898. The method still further comprises identifying a fragment of the test molecule that fits into a cleft in the coactivator binding site that is occupied by the F+5 residue of the peptide. Thus the test molecule preferably interacts with at least one residue selected from the group consisting of: Val 716, Lys 720, Phe 725, Val 730, Gln 733, Ile 737.

Use of an agonist in combination with an inhibitor of coactivator binding also provides a unique strategy for delivering therapeutics that have novel tissue-specific effects. For example, coactivator inhibitors can be designed to bind into the site involved in transcriptional activity only when helix-12 is in its agonist bound state. If such coactivator inhibitors are specific for this site of the androgen receptor, it is possible to selectively inhibit that receptor only in the presence of agonist. This would lead to novel, tissue specific antagonism based on the levels of endogenous agonists because one issue may use a different coactivator from another.

Nuclear Receptor Isoforms

The present invention also is applicable to generating new synthetic ligands to distinguish nuclear receptor isoforms. As described herein, ligands can be generated that distinguish between isoforms, thereby allowing the generation of either tissue specific or function specific synthetic ligands. For instance, GR subfamily members have usually one receptor encoded by a single gene, with the exception that there are two PR isoforms, A and B, translated from the same mRNA by alternate initiation from different AUG codons. This method is especially applicable to the TR subfamily which usually has several receptors that are encoded by two (TR) or three (RAR, RXR, and PPAR) genes or have alternate RNA splicing and such an example for TR is described herein.

There are many uses and advantages provided by the present invention. For example, the methods and compositions described herein are useful for identifying peptides, peptidomimetics or small natural or synthetic organic molecules that modulate nuclear receptor activity. The compounds are useful in treating nuclear receptor-based disorders. Methods and compositions of the invention also find use in characterizing structure/function relationships of natural and synthetic ligands.

These characterizations of coactivator binding to the androgen receptor are supported by the experimental data provided in the examples hereinbelow, which are also intended to illustrate various aspects of the present invention. It is not intended that the examples presented herein limit the scope of the present invention.

### EXAMPLES

### Example 1

Peptide Identification and Preparation

To identify peptides that interact with the androgen receptor, phage display techniques were performed using the AR ligand binding domain. Affinity selection of phage-displayed peptides was carried out using methods similar to those mentioned hereinabove (see Sparks, et al., in Phage Display of Peptides and Proteins, A Laboratory Manual, eds. Kay, B. K., et al., (Academic, San Diego), pp. 227-253, (1996)).

According to such a method, biotinylated AR LBD, obtained by a method of specific in vivo biotinylation of an AviTag peptide (available from Avidity, Denver, Colo.) sequence GLNDIFEAQKIEW (wherein the lysine is specifically biotinylated by coexpressed biotin ligase), fused to AR LBD during protein expression, was immobilized in a streptavidin-coated microtiter well. M13 phage particles distributed among 21 libraries displaying a total of greater than 2×10¹⁰ different random or biased amino acid sequences were added to the immobilized AR LBD and incubated for 3 hrs at 25° C. Unbound phage were washed away, and the bound phage were eluted using pH 2 glycine. The eluted phage were amplified by infecting E. coli cells. The amplified phage were then added to immobilized AR LBD, and the cycle of affinity selection was repeated. Enrichment of phage displaying target-specific peptides was monitored after each round of affinity selection using an anti-M13 antibody conjugated to horseradish peroxidase in an ELISA-type assay. Pools of phage enriched for target-specific peptides were plated for individual plaques. The plaques were picked, the phage amplified, and the phage tested for target-specific binding versus non-specific binding to various control proteins such as hexokinase, alcohol dehydrogenase, β-galactosidase, and streptavidin.

DNA was prepared from target-specific phage, the DNA sequence of the peptide-encoding region was determined, and the peptide sequence was deduced. For each target, the peptide sequences were compared and aligned for common motifs. Phage displaying AR-specific peptides were analyzed for their relative binding affinity for the AR LBD. The phage were serially diluted over a 100-fold range and tested for binding to the AR LBD using a phage ELISA assay. Phage that gave a higher ELISA signal at a lower dilution displayed peptides with a relatively higher affinity for the AR LBD. Based on their relative affinity for AR, peptides sequenced from different sequence clusters were selected for synthesis. The peptides were synthesized with a 5 amino acid linker sequence and a C-terminal biotin.

### Example 2

Protein Expression and Purification

Expression and purification of the androgen receptor ligand binding domain (LBD) was performed essentially as described in Matias, P., et al., J. Biol. Chem., 275 (34): 26164-26171, (2000). The cDNA encoding the androgen receptor LBD was cloned as an in-frame fusion with glutathione S-transferase (GST) in a modified pGEX2t vector (Pharmacia) including a coding sequence providing a flexible linker region between the protein domains. The E. coli strain, BL21 (DE3) STAR, was transformed with the expression vector encoding the GST-AR fusion. Expression of the fusion protein was carried out in a 4.5 L fermentation reactor in 2× YT medium containing 10 μM DHT, and induced with 30 μM IPTG at 15° C. for 16-18 hours. Cell pellets were collected by centrifugation and stored at −80° C. until processed. E. coli cells were lysed in the presence of 0.5 mg/ml lysozyme, Benzonase, 0.5% CHAPS, 1 μM DHT with rocking at room temperature for 30 mins, followed by one freeze-thaw cycle. The cell lysate was mixed by rocking for 10 minutes at room temperature, and cellular debris were removed by centrifugation at 25,000×g for 30 minutes at 4° C.

All purification steps used buffers containing 1 μM DHT. The soluble cell lysate was flowed over 5-6 ml of a Glutathione Sepharose 4 Fast Flow resin filled column. The column material was washed with buffer until non-specifically bound protein was removed. Specifically, bound protein was eluted from the column resin with 15 mM glutathione, and fractions containing GST-AR LBD were collected and pooled. Cleavage of the GST moiety of the fusion protein was carried out by diluting the pooled sample to 1 mg/ml protein with 100 mM HEPES pH 7.2 buffer. Thrombin was added to 10 units/mg of total protein and the sample was incubated at room temperature for 4 hours. Following room temperature incubation, the cleavage reaction proceeded for 16-18 hours at 4° C.

Final purification of the AR LBD was carried out by diluting the material 1:3 and loading it onto a 1 ml Hitrap SP column. The column material was washed with buffer containing 110 mM NaCl until non-specifically bound protein was removed. AR LBD protein was eluted from the column material using buffer containing a gradient of NaCl from 110 to 500 mM NaCl. Fractions containing AR LBD were pooled and concentrated to greater than 4 mg/ml. The final purity of AR LBD was determined to be greater than 90%.

Where applicable throughout the foregoing steps, the following buffers were employed. For GST-AR LBD Fusion: 100 mM Hepes 7.2, 0.15 M NaCl, 10% Glycerol, 0.2 mM TCEP, 0.1% octylglucoside, 1 μM DHT. For eluted AR-LBD: 10 mM Hepes 7.2, 0.2 M NaCl, 10% Glycerol, 0.2 mM TCEP, 0.1% octylglucoside, 1 μM DHT.

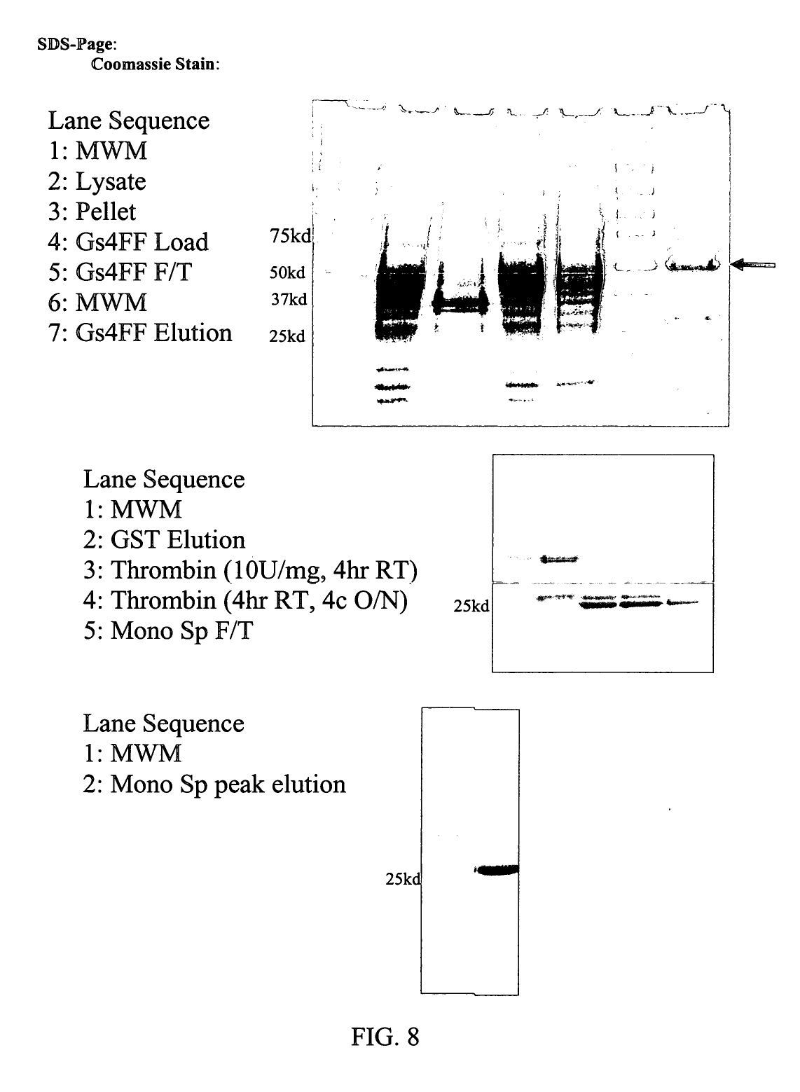

FIG. 8 shows SDS-PAGE data for purification of androgen receptor protein. The first gel shows the initial purification steps over the Glutathione-4 Fast Flow resin. Aliquot samples of soluble E. coli lysate (lane 2), solublized E. coli cellular debris pellet (lane 3), soluble material loaded onto the resin (lane 4), material not binding to resin (lane 5), and pooled fractions specifically eluted with glutathione (lane 7) were electrophoresed using a gradient denaturing gel. The arrow denotes GST-AR LBD fusion protein. The second gel shows the progression of the thrombin cleavage reaction to separate the GST and AR LBD moieties. Lane 2 contains an aliquot sample of the pooled glutathione 4 Fast Flow elution. Lane 3 contains an aliquot sample of partially digested material from lane 2, whereas Lane 4 contains an aliquot sample of completely digested material. Two coomasie-stained protein bands are generated reflecting GST and AR LBD cleaved products. Lane 5 contains material that did not bind to the Mono-S resin, representing GST protein alone. The third gel shows the final purified AR LBD product eluted from the Mono-S resin.

### Example 3

Binding Data for Coactivator Peptides Obtained with Surface Plasmon Resonance Methods

The relative affinities of biotinylated peptides to the AR LBD (bound with DHT) were determined using standard surface plasmon resonance techniques and a Biacore 2000 instrument. 1 mM stock solutions of each synthetic biotinylated peptide in DMSO were diluted 100-fold into HBS-P buffer (0.01 M HEPES pH 7.4, 0.15 M NaCl, 0.005% Surfactant P20) to generate 10 μM working solutions. A four-channel Sensor Chip SA was conditioned according to manufacturer's protocol with three consecutive, 1 minute injections of a solution containing 1 M NaCl and 50 mM NaOH (flowrate of 10 ul/min). After conditioning the streptavidin coated surface, HBS-P buffer was flowed through the cells to achieve a stable baseline prior to immobilization of the biotinylated peptides. To achieve the binding of approximately 250 RU peptides to individual cells, working solutions of peptides were diluted to 100 nM in HBS-P buffer, as follows: 13 μl of peptide CRP_{—}1 in solution was injected to Flowcell 1 at a rate of 5 μl/min generating 240 RU; 10 μl of peptide CRP_{—}3 solution was injected to Flowcell 2 generating 250 RU; 10 μl of peptide CRP_{—}4 solution was injected to Flowcell 3 generating 250 RU; and 10 μl of SMRT2B, a peptide fragment corresponding to amino acid residues 1316-1333 of the coregulatory transcriptional repressor protein SMRT was injected to Flowcell 4 generating 269 RU. Unbound streptavidin sites were blocked by injection of 20 μl of a 1 mM biotin solution to all four Flowcells at a 10 μl/min rate.

SMRT (“silencing mediator of retinoic acid and thyroid hormone receptors”) is a co-repressor protein for nuclear receptors (see, e.g., Chen, J. D., and Evans, R. M., “A transcriptional co-repressor that interacts with nuclear hormone receptors”, Nature, 377, 454-457, (1995)). SMRT has Genbank accession number U37146, and Protein sequence ID number: AAC50236.1, and has sequence

(SEQ ID NO: 26)

MEAWDAHPDKEAFAAEAQKLPGDPPCWTSGLPFPVPPREVIKASPHAPDP
SAFSYAPPGHPLPLGLHDTARPVLPRPPTISNPPPLISSAKHPSVLERQI
GAISQGMSVQLHVPYSEHAKAPVGPVTMGLPLPMDPKKLAPFSGVKQEQL
SPRGQAGPPESLGVPTAQEASVLRGTALGSVPGGSITKGIPSTRVPSDSA
ITYRGSITHGTPADVLYKGTITRIIGEDSPSRLDRGREDSLPKGHVIYEG
KKGHVLSYEGGMSVTQCSKEDGRSSSGPPHETAAPKRTYDMMEGRVGRAI
SSASIEGLMGRAIPPERHSPHHLKEQHHIRGSITQGIPRSYVEAQEDYLR
REKLLKREGTPPPPPPSRDLTEAYKTQALGPLKLKPAHEGLVATVKEAGR
SIHEIPREELRHTPELPLAPRPLKEGSITQGTPLKYDTGASTTGSKKHDV
RSLIGSPGRTFPPVHPLDVMADARALERACYEESLKSRPGTASSSGGSIA
RGAPVIVPELGKPRQSPLTYEDHGAPFAGHLPRGSPVTMREPTPRLQEGS
LSSSKASQDRKLTSTPREIAKSPHSTVPEHHPHPISPYEHLLRGVSGVDL
YRSHIPLAFDPTSIPRGIPLDAAAAYYLPRHLAPNPTYPHLYPPYLIRGY
PDTAALENRQTIINDYITSQQMHHNTATAMAQRADMLRGLSPRESSLALN
YAAGPRGIIDLSQVPHLPVLVPPTPGTPATAMDRLAYLPTAPQPFSSRHS
SSPLSPGGPTHLTKPTTTSSSERERDRDRERDRDREREKSILTSTTTVEH
APIWRPGTEQSSGSSGSSGGGGGSSSRPASHSHAHQHSPISPRTQDALQQ
RPSVLHNTGMKGIITAVEPSKPTVLRSTSTSSPVRPAATFPPATHCPLGG
TLDGVYPTLMEPVLLPKEAPRVARPERPRADTGHAFLAKPPARSGLEPAS
SPSKGSEPRPLVPPVSGHATIARTPAKNLAPHHASPDPPAPPASASDPHR
EKTQSKPFSIQELELRSLGYHGSSYSPEGVEPVSPVSSPSLTHDKGLPKH
LEELDKSHLEGELRPKQPGPVKLGGEAAHLPHLRPLPESQPSSSPLLQTA
PGVKGHQRVVTLAQHISEVITQDYTRHHPQQLSAPLPAPLYSFPGASCPV
LDLRRPPSDLYLPPPDHGAPARGSPHSEGGKRSPEPNKTSVLGGGEDGIE
PVSPPEGMTEPGHSRSAVYPLLYRDGEQTEPSRMGSKSPGNTSQPPAFFS
KLTESNSAMVKSKKQEINKKLNTHNRNEPEYNISQPGTEIFNMPAITGTG
LMTYRSQAVQEHASTNMGLEAIIRKALMGKYDQWEESPPLSANAFNPLNA
SASLPAAMPITAADGRSDHTLTSPGGGGKAKVSGRPSSRKAKSPAPGLAS
GDRPPSVSSVHSEGDCNRRTPLTNRVWEDRPSSAGSTPFPYNPLIMRLQA
GVMASPPPPGLPAGSGPLAGPHHAWDEEPKPLLCSQYETLSDSE

The peptide used herein is a fragment of SMRT having sequence TNMGLEA RKALMGKYD (SEQ ID NO: 27), identified by underlining in the sequence of SMRT.

A kinetic analysis of the interaction between purified AR LBD (DHT) with each of the peptides was performed. Purified AR LBD (DHT) was diluted into HBS-P buffer to a concentration of 10 μM. Then, 60 μL of the 10 μM AR LBD solution was injected to all four Flowcells using the Kinject protocol (contact time was 360 seconds, dissociation time was 360 seconds). Data for the association and dissociation phase were collected and stored for later analysis. Following the dissociation phase, the surface of the chip was regenerated to remove residual AR LBD protein by QuickInject of 10 μl of buffer containing 10 mM HEPES, 50% ethylene glycol, at pH 11. Following the establishment of a stable baseline, the same procedure was repeated using a series of AR LBD (DHT) dilutions (5 μM, 1 μM, and 300 nM) in an iterative manner.

Analysis of the data was performed using BIAevaluation 3.0 software by fitting curves to standardized data. The SMRT2B signals were subtracted as background from the three remaining peptide signals, and curves for the dilutions series were fit using standard methods (e.g., assuming a Langmuir binding model). Estimates of the relative binding affinity for each peptide were calculated using BIAevaluation software curve fitting based on the Marquardt-Levenberg algorithm (J. W. Wells, in Receptor-Ligand Interactions, A Practical Approach, ed., E. C. Hulme).

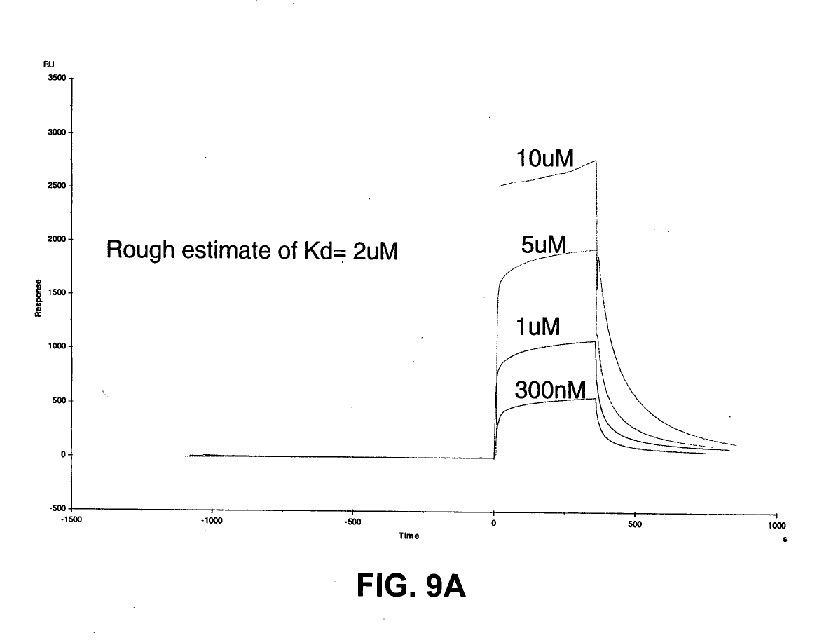

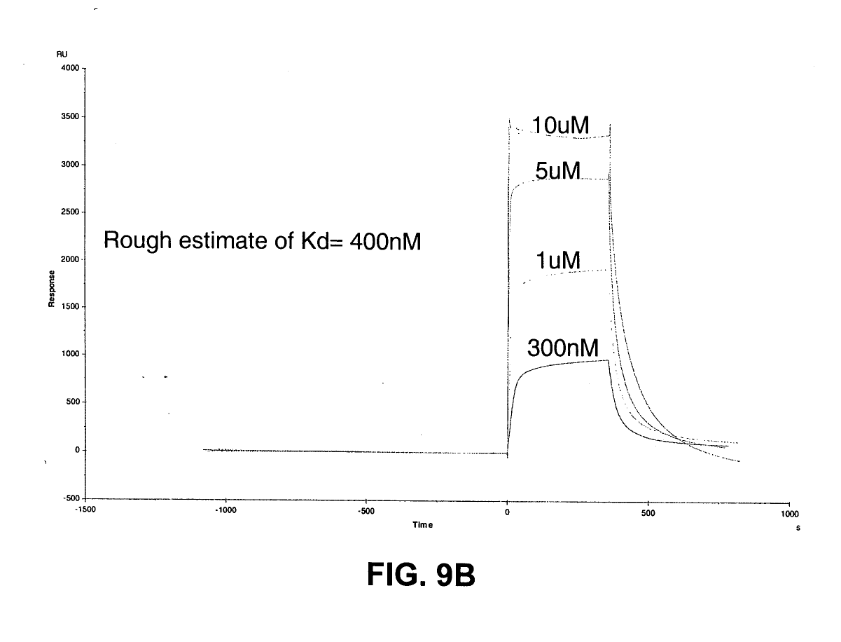

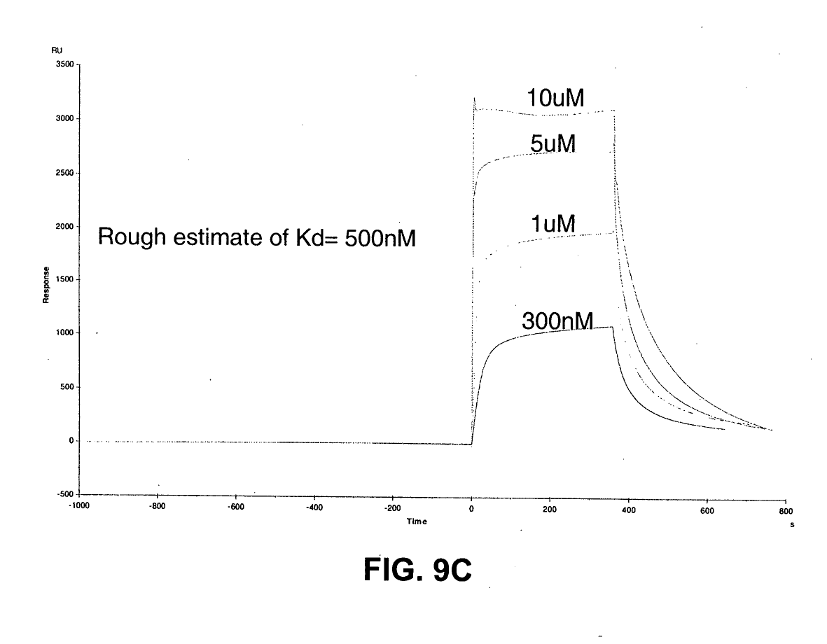

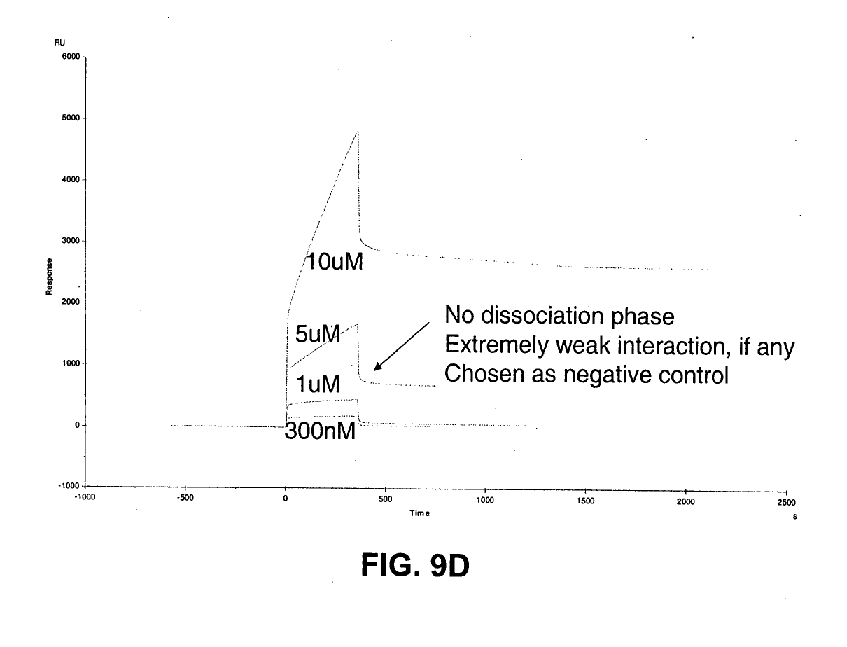

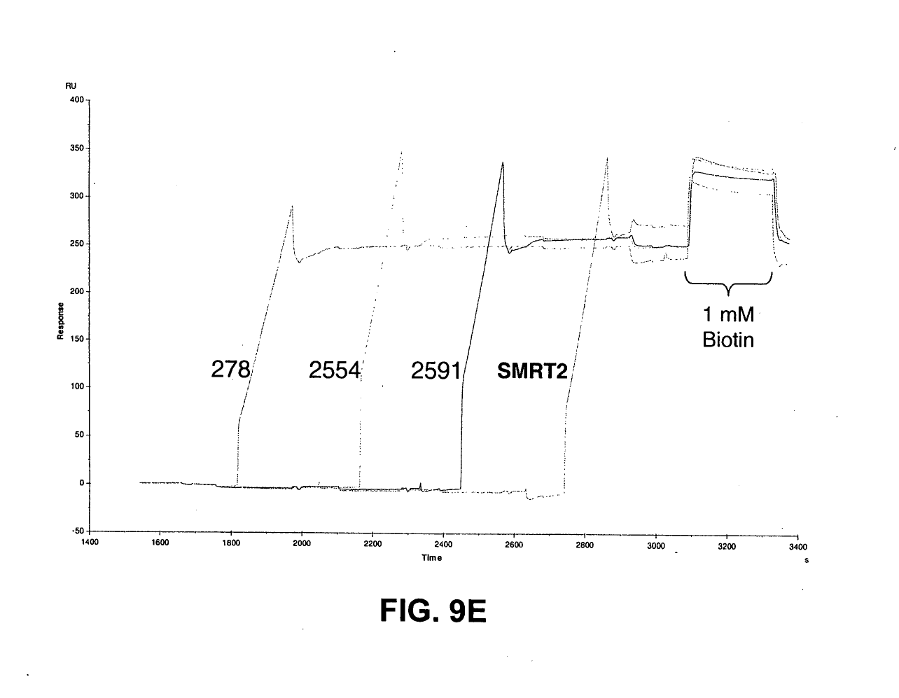

FIGS. 9A, 9B, 9C, and 9E display overlay plots of 4 different concentrations of AR LBD protein (10, 5, 1 and 0.3 μM, respectively) interacting with peptides CRP_{—}1, CRP_{—}3, CRP_{—}4, and SMRT2B, respectively. The overlay plots depict the relative unit response as measured by surface plasmon resonance over time. Association phase of AR LBD with each peptide precedes the dissociation phase as depicted in FIG. 9E. FIG. 9D displays the relative unit response of each of the four biotinylated peptides as they bind irreversibly to distinct streptavidin coated flow cell channels. Each peptide generated approximately 250-300 relative units. The estimated values for K_{d} were calculated using BIAevaluation 3.0 software and assumed Langmuir binding.

### Example 4

Crystallization and Data Collection for Complexes of AR with Coactivator-Derived Peptides

The complexes of coactivator-derived peptide and AR LBD were prepared by mixing at 0° C., for a period of 2 hours, variable ratios of coactivator peptide (3 to 10 mM) and protein (at about 4 mg/ml). Crystals were obtained by vapor diffusion method referred to as “sitting drop”, using different crystal screens and improved with several additives. Frozen crystals were measured at a beam line at ALS (Lawrence Berkeley Laboratory). The crystals belong to space group P212121 (orthorhombic) and contain one molecule per asymmetric unit. The diffraction data was integrated with Denzo, and scaled using Scalepack (Otwinowski, Z., and Minor, W. “Processing of X-ray diffraction data collected in oscillation mode”, Methods in Enzymology, 276:307-326, (1997)).

The structure determination for AR LBD-coactivator peptide complexes was facilitated by using the atomic coordinates available for the AR LBD (see, Sack, J. S., Kish, K. F., Wang, C., Attar, R. M., Kiefer, S. E., An, Y., Wu, G. Y., Scheffler, J. E., Salvati, M. E., Krystek Jr., S. R., Weinmann, R., Einspahr, H. M., “Crystallographic Structure of the Ligand-Binding Domains of the Androgen Receptor and its T877A Mutant Complexed with the Natural Agonist Dihydrotestosterone”, Proc. Nat. Acad. Sci. USA, 98, 4904-4909, (2001); and Matias, P. M., Donner, P., Coelho, R., Thomaz, M., Peixoto, C., Macedo, S., Otto, N., Joschko, S., Scholz, P., Wegg, A., Basler, S., Schafer, M., Egner, U., Carrondo, M. A., “Structural Evidence for Ligand Specificity in the Binding Domain of the Human Androgen Receptor. Implications for Pathogenic Gene Mutations”, J. Biol. Chem., 275:26164-26171, (2000)) by molecular replacement using the program AMoRe (Navaza, J., “An automated package for molecular replacement”, Acta Crystallographica, A50:157-163, (1994)).

After rigid body refinement of the AR LBD molecule(s), electron density maps were calculated and fit. Electron density corresponding to the coactivator peptides could clearly be seen in the first calculated maps. The electron density for the peptide was modelled as a short α-helix. Final refinement steps were carried out with the program CNS (Brünger, A. T.; Adams, P. D.; Clore, G. M., DeLano, W. L., Gros, P., Grosse-Kuntsleve, R. W., Jiang, J. S., Kuszewski, J., Nilges, M., Pannu, N. S., “Crystallography and NMR system: a new software suite for macromolecular structure determination”, Acta Crystallographica, D54, 905-921, (1998)) interspersed with manual rebuilding on a SGI graphics workstation using the program QUANTA, monitored using the free-R factor. The models presented herein comprise continuous electron density for the entire AR LBD and for nearly the entire length of the coactivator peptides bound thereto. More than 99% of all residues fall into the most favored or additionally favored regions of peptide side-chain conformational space (as calculated with the program PROCHECK).

Table 1 entitled “Structures of AR LBD with DHT and a coactivator-derived peptide” presented herewith on CD-R in a file named Table1_ARLBD_DHT_CDP.txt, contains the coordinates, in PDB file format, for two refined cocrystal structures. Table 1 comprises two parts, (A) and (B), each of which contains a set of coordinates of a single complex. Appendices 6Z and 7Z, herein, respectively present header information from each of the two PDB files. In the PDB files in Table 1, the AR LBD, a coactivator peptide, the ligand DHT, and crystallographic waters are given the chain designators A, P, L, and S, respectively. The atoms of the residues in the AR LBD and the coactivator peptide are identified by standard 3-letter designations. The ligand atoms are identified as “DHT”, and the water molecules are designated variously as “TIP”, and “HOH”. The data in the PDB files is presented in columns which contain the following information, in order: the card identifier (“ATOM” or “END”); the Atom number; the atom type, specifying both the element symbol and the position in the residue; the 3-letter abbreviation of the residue; a chain identifier (A, P, L, S); a residue number; 3 atomic coordinates, x, y, z, in order; a number representing the atom occupancy, given by a value of 1 if the atom is seen in the electron density, and a value of 0 if it has been built; a number representing the B-factor of the atom; and the chain identifier presented a second time.

In Table 1, at (A), is found the structure for AR LBD, comprising residues 669-918 of AR, bound to the ligand DHT, and an ARA70-derived coactivator peptide, with 106 crystallographic waters. The coactivator peptide is found at residues 920-930, inclusive, and is a 15-mer with sequence RETSEKFKLLFQSYN (SEQ ID NO: 13) that contains the FXXLF motif. Only the middle 11 residues of the coactivator, from the first S to Y, can be seen clearly in the electron density; the terminal N residue can also be seen faintly but is not shown in the PDB file. The ligand DHT is designated “residue” 931, and the 106 waters are labeled residues 1-106. The first 101 are designated OH2 TIP, and the remainder are labeled “O HOH”. This structure is solved at a resolution of 2.3 Å.

In Table 1, at (B), is found the structure for AR LBD, comprising residues 669-918 of AR, bound to the ligand DHT, and a Grip1-box 3-derived peptide, with 160 crystallographic waters. The coactivator peptide is found at residues 920-931, inclusive, and is a 14-mer with sequence KENALLRYLLDKDD (SEQ ID NO: 14) that contains the LXXLL motif. Only the last 13 residues of the coactivator can be seen in the electron density, i.e., not including the K; the second residue, Glutamate, is shown as an alanine because the side-chain could not be accurately modeled. The ligand DHT is designated “residue” 932, and the 160 crystallographic waters are labeled residues 1-160. The first 156 waters are designated OH2 TIP, and the remainder are labeled “O HOH”. This structure is solved at a resolution of 2.07 Å.

Tables 3A and 3B contain crystallographic data for two cocrystals of AR with coactivator-derived peptides. The terms and symbols used in Tables 3A and 3B would be understood without further explanation to a crystallographer of ordinary skill in the art.

TABLE 3A
Crystallographic data for co-crystals of AR with DHT and
coactivator-derived peptides.
Summary of Crystallographic Statistics for coordinates from
minimization and B-factor refinement

| | Coactivator derived from | |
|---|---|---|
| | ARA70 | Grip-1 box 3 |
| | 15-mer containing | 14-mer containing |
| | FXXLF motig | LXXLL motif |
| Data Collection | | |
| No. molecules in | 1 | 1 |
| asymmetric unit | | |
| Space group | P2(1)2(1)2(1) | P2(1)2(1)2(1) |
| Unit Cell dimensions: | a = 55.680 | a = 54.49 |
| | b = 66.423 | b = 67.37 |
| | c = 68.253 | c = 70.52 |
| | α = 90°; β = 90°; | α = 90°; β = = 90°; |
| | γ = 90°. | γ = 90°. |
| Resolution Range | 24-2.3 Å | 24-2.07 Å |
| Reflections Measured | 458173 | 393765 |
| Unique Reflections | 13713 | 16416 |
| Completeness (%) | | |
| Overall | 92.8 | 97.2 |
| Outermost Shell | 85.2 | 94.3 |
| Refinement | | |
| Reflections used in | 10881 | 15915 |
| refinement | | |
| Resolution | 2.3 Å | 2.07 Å |
| R merge (%)^{a} | 5 | 4.4 |
| Rfactor (%)^{b} | 22.8 | 19.8 |
| Rfree (%)^{c} | 25.8 | 23.2 |
| Bond r.m.s. deviation (Å) | 0.008 | 0.007 |
| No. of water molecules | 106 | 160 |

$\begin{matrix}{}^{a} R merge ( \% ) = {\sum }_{hkl} \vert < I > - I \vert / {\sum }_{hkl} \vert I \vert \\ {}^{b} R factor ( \% ) = {\sum }_{hkl} \vert \vert Fo \vert - \vert Fc \vert \vert / {\sum }_{hkl} \vert Fo \vert \\ {}^{c} R free set contained 5 \% of total data .\end{matrix}$

TABLE 3B
Crystallographic data for co-crystals of AR with DHT and
coactivator-derived peptides.

| Remark | ARA70-derived | GRIP Box3-derived |
|---|---|---|
| Starting r | 0.2287 | 0.2192 |
| Free_r | 0.2639 | 0.2461 |
| Final r | 0.2274 | 0.2127 |
| Free_r | 0.2645 | 0.2377 |
| B (rmsd) | | |
| For bonded mainchain atoms | 1.240 | 1.444 |
| Target | 1.5 | 1.5 |
| For bonded sidechain atoms | 1.967 | 2.318 |
| Target | 2.0 | 2.0 |
| For angle mainchain atoms | 2.079 | 2.384 |
| Target | 2.0 | 2.0 |
| For angle sidechain atoms | 3.052 | 3.426 |
| Target | 2.5 | 2.5 |
| Target (steps) | mlf | mlf |
| Final wa | 4.65626 | 1.56885 |
| Final rweight | 0.0885 | 0.0724 |
| Wa | (4.65626) | (1.56885) |
| Md-method | torsion | torsion |
| Annealing schedule | constant | constant |
| Starting temperature | 1600 | 1600 |
| Total md steps | 1 * 100 | 1 * 100 |
| Cycles | 2 | 2 |
| Coordinate steps | 20 | 20 |
| B-factor steps | 10 | 10 |
| B correction resolution | 6.0-2.3 | 6.0-2.07 |
| Initial B-factor correction | | |
| applied to fobs: | | |
| B11 | −25.343 | −7.830 |
| B22 | 14.695 | 3.046 |
| B33 | 10.648 | 4.783 |
| B12 | 0.000 | 0.000 |
| B13 | 0.000 | 0.000 |
| B23 | 0.000 | 0.000 |
| B-factor correction applied to | −0.154 | 0.794 |
| coordinate array B | | |
| Bulk solvent: density level e/A³ | 0.317003 | 0.368405 |
| B-factor A² | 26.8235 | 55.156 |
| Theoretical total number | 11733 (100.0%) | 16360 (100.0%) |
| of reflections in | | |
| resolution range | | |
| Number of unobserved | 852 (7.3%) | 445 (2.7%) |
| reflections | | |
| (no entry or \|F\| = 0) | | |
| Number of reflections rejected | 0 (0.0%) | 0 (0.0%) |
| Total number of reflections used | 10881 (92.7%) | 15915 (97.3%) |
| Number of reflections in | 10321 (88.0%) | 15136 (92.5%) |
| working set | | |
| number of reflections in test set | 560 (4.8%) | 779 (4.8%) |

In preparing the data in Table 3B, reflections with |Fobs|/σ_F<0.0, and reflections with |Fobs|>10000×rms(Fobs) were rejected.

### Example 5

Crystallization and Data Collection for Complexes of AR with Coactivator-Related Peptides

Purified AR LBD at 4.5 mg/mL was combined with a 3× molar excess of peptide and allowed to complex at least 1 hour before crystallization trials. The AR-peptide complexes were crystallized using the hanging drop vapor diffusion method by combining the protein-peptide solution in a 1:1 ratio with a well solution containing 0.6-0.8M sodium citrate and 100 mM Tris or HEPES buffer pH 7-8. The addition of ethylene glycol to a well concentration of 8% improved crystal quality. Crystals typically appeared after one to two days, with maximal size being attained within 2 weeks. The crystals were harvested into a cryo-protectant solution consisting of well solution plus 10% glycerol before being flash frozen in liquid nitrogen. Data sets were collected at the Advanced Light Source beamline 8.3.1 at the Lawrence Berkeley Laboratory and processed using the software programs, Denzo and Scalepack (Otwinowski, Z., and Minor, W., “Processing of X-ray Diffraction Data Collected in Oscillation Mode”, in Methods in Enzymology: Macromolecular Crystallography, part A, C. W. Carter, and R. M. Sweet, (eds.) (New York, Academic Press), 307-326, (1997)). Molecular replacement searches were performed with CNS (Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al., “Crystallography & NMR system: A new software suite for macromolecular structure determination”, Acta Crystallogr. D Biol Crystallogr., 54 (Pt. 5), 905-921, (1998)) Initial searches for AR-CRP_{—}3 were performed using the structure of AR-R1881 (PDB identifier 1E3G) as a search model. Subsequent searches for all other complexes were performed using the refined structure of AR-CRP_{—}3 as a model. Refinement of all structures were performed with CNS.

In general, for coactivator-related peptides, typically only the hydrophobic motif, LXXLL, etc., is ordered in the crystal structure. This is because the portion of the peptide sequence outside of the motif is not in any way optimized for binding against the rest of the receptor structure away from the key interactions within the coactivator binding site.

Table 2, entitled “Structures of AR LBD with DHT and a coactivator-related peptide” presented herewith on CD-R in a file named Table2_ARLBD_DHT_CRP.txt, contains the coordinates, in PDB file format, for eight refined cocrystal structures. Table 2 comprises eight parts, (A)-(H), each of which contains a set of coordinates of a single complex. Appendices 1Z-8Z, herein, respectively present header information from each of the eight PDB files. In the PDB files presented in Table 2, the AR LBD, a coactivator peptide, the ligand DHT, and crystallographic waters are given the chain designators A, P, L, and S, respectively. The atoms of the residues in the AR LBD and the coactivator peptide are identified by standard 3-letter designations. The ligand atoms are identified as “DHT”, and the water molecules are designated “HOH”. The data in the PDB files is presented in columns which contain the following information, in order: the card identifier (“ATOM” or “END”); the Atom number; the atom type, specifying both the element symbol and the position in the residue; the 3-letter abbreviation of the residue; a chain identifier (A, P, L, S); a residue number; 3 atomic coordinates, x, y, z, in order; a number representing occupancy; a number representing B-factor; and the chain identifier (which is presented a second time because certain computer programs require it to be in different places in the PDB format).

In Table 2, at (A), is found the structure for AR LBD, comprising residues 669-918 of AR, bound to the ligand DHT, and a coactivator-related peptide CRP_{—}1, with 162 crystallographic waters. The coactivator peptide is a 15-mer with sequence SSRGLLWDLLTKDSR (SEQ ID NO: 6) that contains the LXXLL motif. The peptide is found at residues 99-106 (with chain identifier “P”), inclusive, where only the middle 8 residues of the coactivator, from the glycine to threonine, can be seen clearly in the electron density. The ligand DHT is designated “residue” 200 (chain “L”), and the 162 waters are labeled residues 1-162 (with chain identifier, “S”). This structure is solved at a resolution of 1.6, Å.

In Table 2, at (B), is found the structure for AR LBD, comprising residues 669-918 of AR, bound to the ligand DHT, and a coactivator-related peptide CRP_{—}2, with 64 crystallographic waters. The coactivator peptide is a 15-mer with sequence: found at residues 99-106, inclusive, where only the middle 8 residues of the coactivator, from the first serine to the first aspartate, can be seen clearly in the electron density. The ligand DHT is designated “residue” 200, and the 64 waters are labeled residues 1-64. Additionally, there are two species, “EDO” (ethylene glycol) labeled residue 201, and “SO₄” (sulfate) labeled residue 202. This structure is solved at a resolution of 2.2 Å.

In Table 2, at (C), is found the structure for AR LBD, comprising residues 669-918 of AR, bound to the ligand DHT, and a coactivator-related peptide CRP_{—}3, with 201 crystallographic waters. The coactivator peptide is a 15-mer with sequence SSRFESLFAGEKESR (SEQ ID NO: 8) that contains the FXXLF motif. The peptide is found at residues 98-107 (with chain identifier “P”), inclusive, where only the middle 10 residues of the coactivator, from the first serine to glycine, can be seen clearly in the electron density. The ligand DHT is designated “residue” 200 (chain identifier “L”), and the 201 waters are labeled residues 1-201, with chain identifier “S”. This structure is solved at a resolution of 1.45 Å.

In Table 2, at (D), is found the structure for AR LBD, comprising residues 669-918 of AR, bound to the ligand DHT, and a coactivator-related peptide CRP_{—}4, with 145 crystallographic waters. The coactivator peptide is a 15-mer with sequence SSKFAALWDPPKLSR (SEQ ID NO: 9) that contains the FXXLW motif. The peptide is found at residues 99-106 (with chain identifier “P”), inclusive, where only the middle 8 residues of the coactivator, from the first serine to aspartate, can be seen clearly in the electron density. The ligand DHT is designated “residue” 200, and the 145 waters are labeled residues 1-145, with chain identifier “S”. This structure is solved at a resolution of 1.8 Å.

In Table 2, at (E), is found the structure for AR LBD, comprising residues 669-918 of AR, bound to the ligand DHT, and a coactivator-related peptide CRP_{—}5, with 75 crystallographic waters. The coactivator peptide is a 15-mer with sequence: SRFADFFRNEGLSGSR (SEQ ID NO: 10) that contains the FXXFF motif. The peptide is found at residues 99-106, inclusive, where only the 8 residues of the coactivator, from the first serine to the first arginine, can be seen clearly in the electron density. The ligand DHT is designated “residue” 200, and the 75 waters are labeled residues 1-75. This structure is solved at a resolution of 2.2 Å.

In Table 2, at (F), is found the structure for AR LBD, comprising residues 670-918 of AR, bound to the ligand DHT, and a coactivator-related peptide CRP_{—}6, with 157 crystallographic waters. The coactivator peptide is a 15-mer with sequence: SSNTPRFKEYFMQSR (SEQ ID NO: 11) that contains the FXXYF motif. The peptide is found at residues 96-108, inclusive, where only the 13 residues of the coactivator, from the second serine to the last serine, can be seen clearly in the electron density. The ligand DHT is designated “residue” 200, and the 157 waters are labeled residues 1-157. This structure is solved at a resolution of 1.6 Å.

In Table 2, at (G), is found the structure for AR LBD, comprising residues 670-918 of AR, bound to the ligand DHT, and a coactivator-related peptide CRP_{—}7, with 88 crystallographic waters. The coactivator peptide is a 15-mer with sequence: SRWAEVWDDNSKVSR (SEQ ID NO: 12) that contains the WXXVW motif. The peptide is found at residues 99-107, inclusive, where only the 9 residues of the coactivator, from the first serine to the first aspartic acid, can be seen clearly in the electron density. The ligand DHT is designated “residue” 200, and the 88 waters are labeled residues 1-88. This structure is solved at a resolution of 2.1 Å.

In Table 2, at (H), is found the structure for AR LBD, comprising residues 671-918 of AR, bound to the ligand DHT, and a coactivator-related peptide CRP_{—}8, with 101 crystallographic waters. The coactivator peptide is a 15-mer with sequence: SSEVTGMRFRDLFSR (SEQ ID NO: 24) that contains the FXXLF motif. The peptide is found at residues 99-105, inclusive, where only 7 residues of the coactivator can be seen clearly in the electron density. The ligand DHT is designated “residue” 200, and the 101 waters are labeled residues 1-101. This structure is solved at a resolution of 2.1 Å. Residues flanking the 3 core hydrophobic residues of F101, L104, and F105 were left as alanines due to the absence of side chain density. Otherwise, interactions are largely the same as for CRP_{—}3.

TABLE 4A
Crystallographic data for selected co-crystals of AR with DHT and coactivator-related peptides.

| Remark | CRP_1 | CRP_2 | CRP_3 | CRP_4 | CRP_5 |
|---|---|---|---|---|---|
| Refinement resolution (Å) | 20.0-1.6 | 20.0-2.2 | 20.0-1.45 | 20.0-1.8 | 20.0-2.2 |
| Starting r | 0.2017 | 0.2096 | 0.1954 | 0.2016 | 0.1996 |
| Free_r | 0.2220 | 0.2488 | 0.2034 | 0.2380 | 0.2471 |
| Final r | 0.1989 | 0.2083 | 0.1953 | 0.2007 | 0.1994 |
| Free_r | 0.2197 | 0.2473 | 0.2033 | 0.2350 | 0.2464 |
| Rmsd bonds | 0.005787 | 0.007093 | 0.005800 | 0.006350 | 0.006854 |
| Rmsd angles | 1.06472 | 1.13564 | 1.09832 | 1.05669 | 1.05874 |
| B (rmsd) | | | | | |
| For bonded mainchain atoms | 1.185 | — | 1.101 | 1.265 | 1.445 |
| Target | 1.5 | — | 1.5 | 1.5 | 1.5 |
| For bonded sidechain atoms | 2.132 | — | 2.209 | 2.139 | 2.249 |
| Target | 2.0 | — | 2.0 | 2.0 | 2.0 |
| For angle mainchain atoms | 1.810 | — | 1.699 | 1.853 | 2.355 |
| Target | 2.0 | — | 2.0 | 2.0 | 2.0 |
| For angle sidechain atoms | 3.187 | — | 3.220 | 3.264 | 3.242 |
| Target | 2.5 | — | 2.5 | 2.5 | 2.5 |
| Rweight | 0.1000 | 2.4098 | 0.1000 | 0.1000 | 0.1000 |
| Wa | 0.794839 | — | 0.53362 | 1.28071 | 2.52582 |
| Target (steps) | mlf (30) | mlf (30) | mlf (30) | mlf (30) | mlf (30) |
| Space group | P2(1)2(1)2(1) | P2(1)2(1)2(1) | P2(1)2(1)2(1) | P2(1)2(1)2(1) | P2(1)2(1)2(1) |
| A | 54.225 | 55.947 | 55.407 | 55.826 | 54.323 |
| B | 66.327 | 66.434 | 66.200 | 66.128 | 66.592 |
| C | 69.407 | 71.720 | 68.832 | 68.381 | 70.223 |
| A | 90 | 90 | 90 | 90 | 90 |
| B | 90 | 90 | 90 | 90 | 90 |
| Γ | 90 | 90 | 90 | 90 | 90 |
| Ncs | none | none | none | none | none |
| B-correction resolution | 6.0-1.6 | 6.0-2.2 | 6.0-1.45 | 6.0-1.8 | 6.0-2.2 |
| Initial B-factor correction | | | | | |
| applied to fobs: | | | | | |
| B11 | −9.002 | −32.766 | −9.249 | −14.446 | −14.285 |
| B22 | 5.334 | 18.474 | 6.274 | 7.925 | 6.754 |
| B33 | 3.668 | 14.292 | 2.975 | 6.521 | 7.531 |
| B12 | 0.000 | 0.000 | 0.000 | 0.000 | 0.000 |
| B13 | 0.000 | 0.000 | 0.000 | 0.000 | 0.000 |
| B23 | 0.000 | 0.000 | 0.000 | 0.000 | 0.000 |
| B-factor correction applied to | −0.051 | −0.532 | −0.003 | 0.051 | −0.076 |
| coordinate array B | | | | | |
| bulk solvent: density level (e/A³) | 0.411252 | 0.365634 | 0.395754 | 0.373424 | 0.367359 |
| B-factor (A²) | 53.0348 | 54.1419 | 50.2566 | 50.5977 | 47.4855 |
| Theoretical total no. of | 33690 (100%) | 14088 (100%) | 45540 (100%) | 24048 (100%) | 13432 (100%) |
| reflections in resolution range | | | | | |
| No. of unobserved reflections | 953 (2.8%) | 741 (5.3%) | 287 (0.6%) | 277 (1.2%) | 100 (0.7%) |
| (no entry or \|F\| = 0) | | | | | |
| No. of reflections rejected | 0 (0.0%) | 0 (0.0%) | 0 (0.0%) | 0 (0.0%) | 0 (0.0%) |
| Total no. of reflections used | 32737 (97.2%) | 13347 (94.7%) | 45253 (99.4%) | 23771 (98.8%) | 13332 (99.3%) |
| No. of reflections in working | 31107 (92.3%) | 12669 (89.9%) | 43006 (94.4%) | 22619 (94.1%) | 12653 (94.2%) |
| set | | | | | |
| No. of reflections in test set | 1630 (4.8%) | 678 (4.8%) | 2247 (4.9%) | 1152 (4.8%) | 679 (5.1%) |

In each of the structures of Table 4A, reflections with |F_{obs}|/σ_F<0.0, and reflections with |F_{obs}|>10,000×rms(F_{obs}) were rejected.

### Example 6

Structures of Coactivator Related Peptides and the AR:coactivator Interface

The structures of cocrystals of AR with the following coactivator-related peptides from a phage display were obtained: CRP_{—}2 (WXXLF), CRP_{—}3 (FXXLF), CRP_{—}1 (LXXLL), CRP_{—}4 (FXXLW), CRP_{—}5 (FXXFF), CRP_{—}6 (FXXYF), CRP_{—}7 (WXXVW), and CRP_{—}8 (FXXLF). The structures reveal that these hydrophobic motifs bind in a manner analogous to those previously observed in other nuclear receptors that bind to LXXLL p 160 coactivator motifs in that the core hydrophobic motif forms a short helix which binds in a groove formed by coactivator binding site helices 3, 4, 5, and 12 (see, e.g., FIG. 5A, depicting CRP_{—}3 (FXXLF), green; CRP_{—}1 (LXXLL), yellow; CRP_{—}4 (FXXLW), violet). However, there are differences in binding that are unique to the AR LBD and that are revealed by analysis of the various crystal structures.

The binding of the coactivator-related peptides to the AR coactivator binding site buries a region of predominantly hydrophobic surface area from both molecules. The amount of buried surface area is as follows: CRP_{—}3 (FxxLF): 1017.64 A²; CRP_{—}1 (LxxLL): 987.293 A²; CRP_{—}4(FxxLW): 929.731 Å²; CRP_{—}2 (WxxLF): 963.142 Å²; CRP_{—}7 (WxxVW): 881.864 Å²; CRP_{—}5 (FxxFF): 919.738 Å²; CRP_{—}6 (FxxYF): 1229.3 Å².

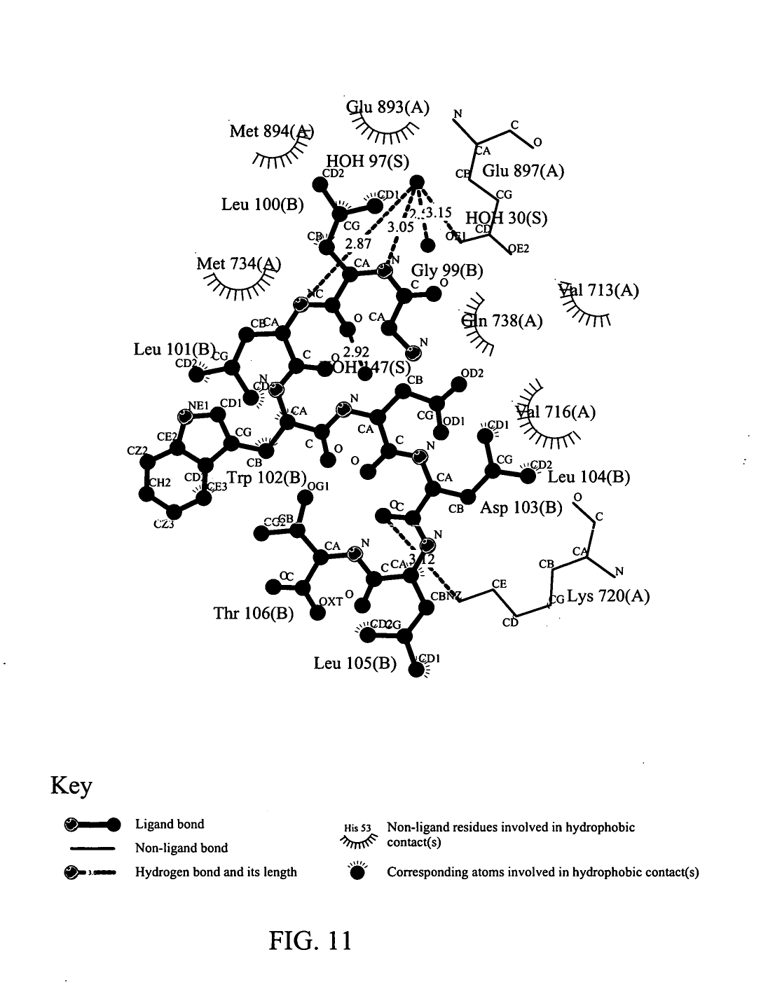

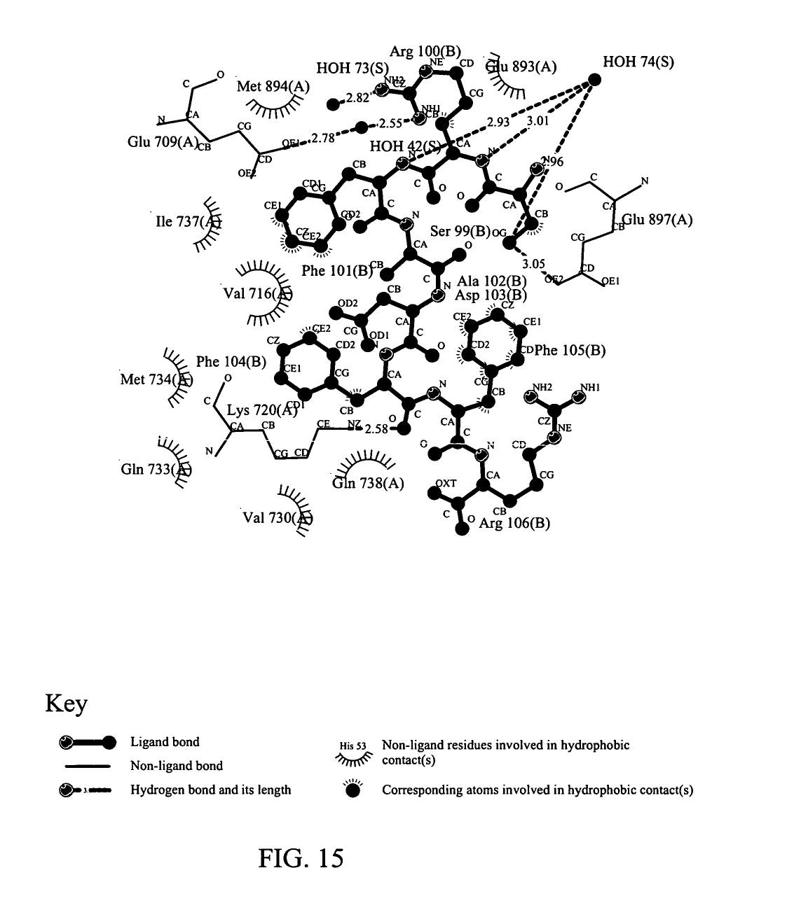

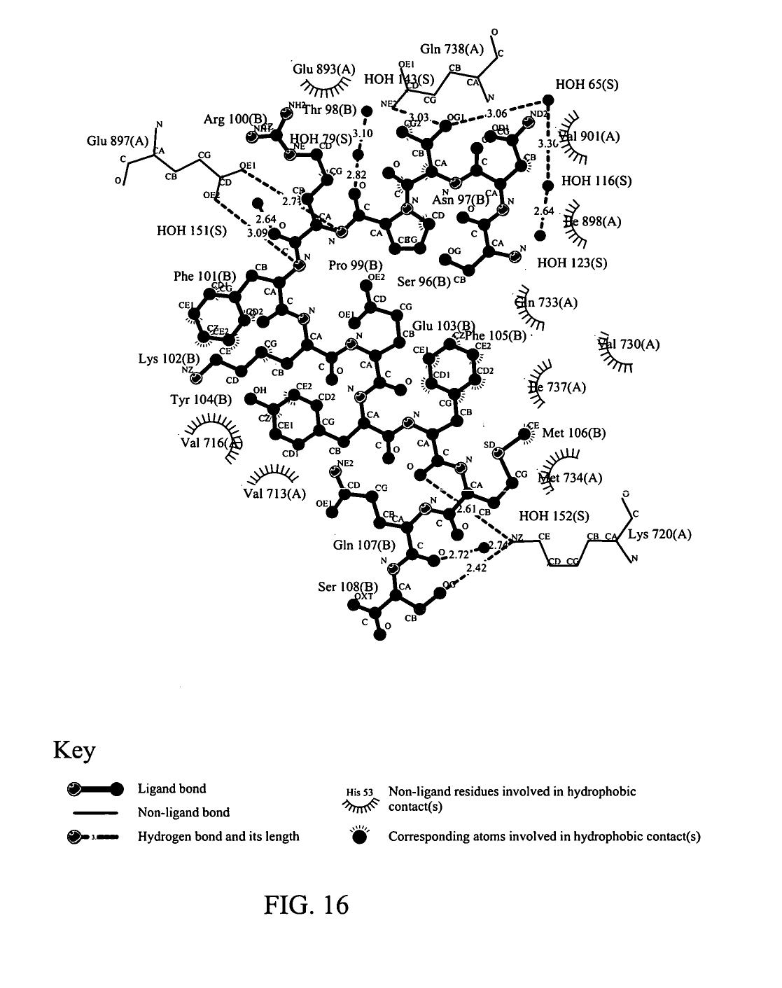

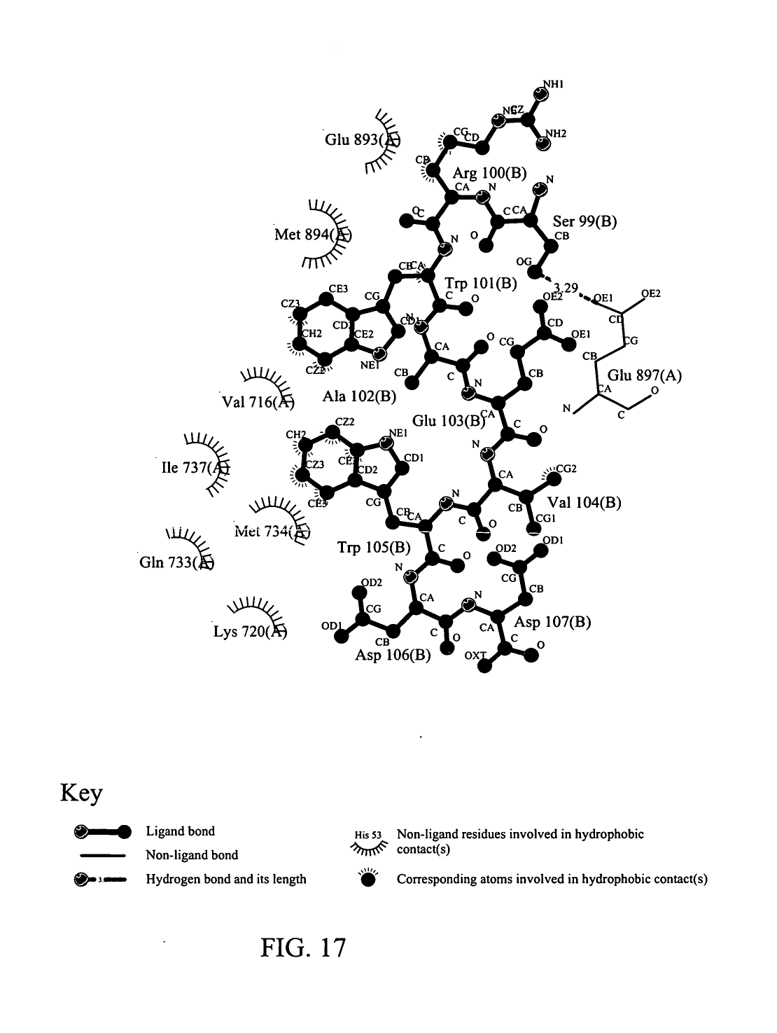

FIGS. 11 and 12-17 illustrate the binding interactions for each peptide. It is to be assumed that if an interaction that is otherwise considered significant is not depicted in any of FIGS. 11-17, then that may be because the residue in question lies at a distance just slightly outside of a threshold employed by the drawing program and therefore is not shown.

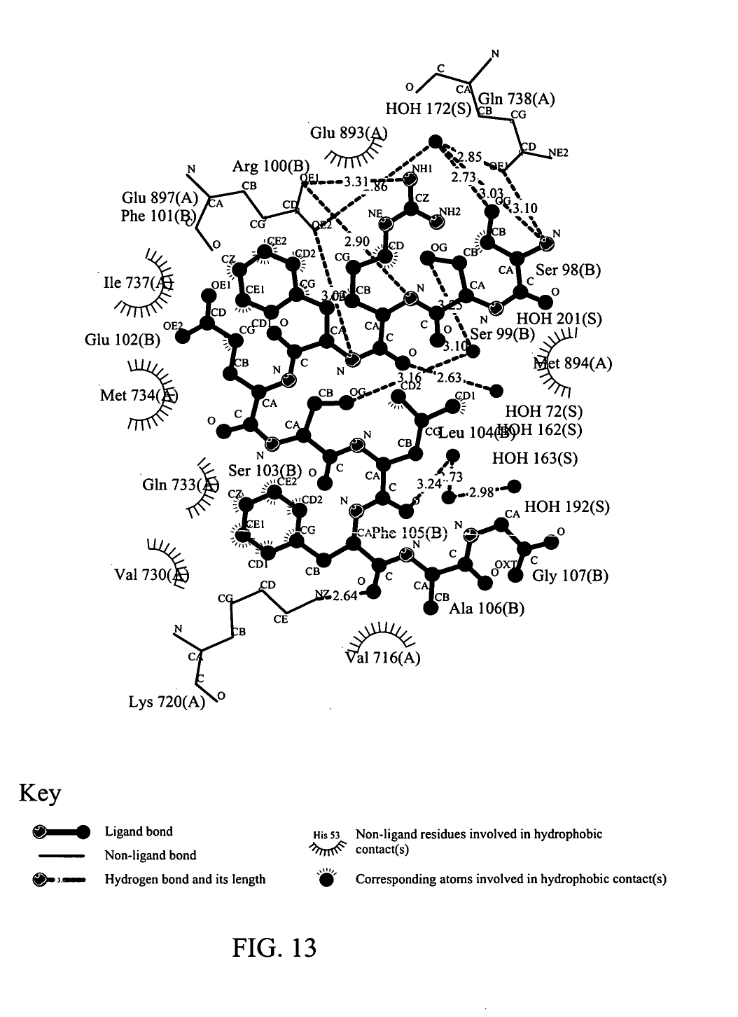

Considering the peptides themselves, CRP_{—}3 (FXXLF) forms a short amphipathic helix which binds on a surface formed by helices 3, 4, 5, and 12 of the AR-LBD. Interactions between the LBD and CRP_{—}3 are predominantly hydrophobic in nature. Phe+1 binds in a wide pocket formed on the bottom by Ile898 (not shown in the FIGs) and on the sides by Met894, Gln738, Met734, Val716, and Leu712 (not shown in the FIGs). The much narrower +5 pocket consists of Ile737 on the bottom and Met734, Gln733, Val730, Phe725, Lys720, and Val716 on the sides. Phe 725 forms only a small part of the surface for the +5 pocket, specifically by forming the top of the +5 binding pocket. It is probably a little too far away to make a strong interaction with a coactivator peptide sequence, and thus is not explicitly shown in FIG. 13. The bulk of the interactions in this pocket derive from Met734 and the aliphatic portion of Lys720, which interact with opposite faces of the benzyl ring of Phe+5. Leu+4 binds in a shallow cleft consisting of Val716, Val713, Leu712, and Met894. The main polar interactions with CRP_{—}3 involve the highly conserved “charge clamp” residues Lys720 and Glu897, which interact with main chain carbonyl and amide groups at opposite ends of the peptide helix.

Hydrophobic interactions between the LBD and hydrophobic residues of the other peptides are largely the same as in CRP_{—}3. The largest differences occur in the CRP_{—}1 (LXXLL) complex where Met734 makes a dramatic shift of about 2.5 Å toward the +1 pocket to accommodate the Leu+1 residue, thereby widening the +5 pocket. The position of the Met734 residue in this complex also allows it to make a hydrophobic interaction with Trp+2 of CRP_{—}1.

Interactions between the core hydrophobic motif residues of CRP_{—}6 (FXXYF) and the AR-LBD are the same as for the other coactivator-related peptides described herein. CRP_{—}6 however makes a number of interactions involving residues flanking the core hydrophobic motif. CRP_{—}6 was the most ordered out of all coactivator-related peptides crystallized to date, with 13 out of 15 peptide residues observed in the electron density. Thr−3 binds in a pocket formed by Glu897, Ile898, Val901, and Gln738, which hydrogen bonds with the hydroxyl group of Thr−3. Lys+2 makes a water mediated hydrogen bond to Asp731 on helix 5. Met+6 makes hydrophobic contacts in a small indentation formed between Val730 and Met734. Ser+8 makes a hydrogen bond to Lys720.

The majority of differences between complexes of the AR coactivator binding site and various coactivator peptides lie in the nature of their polar interactions. Only the CRP_{—}3 peptides with the FXXLF motif forms interactions via their main chain amide nitrogens with the charge clamp residue, Glu 897. Comparisons with the other peptide complexes reveal that the bound positions of the other peptides are skewed in a manner such that Glu897 is too far away to interact with peptide main chain atoms. For example, CRP_{—}1 (LXXLL) and CRP_{—}4 (FXXLW) are displaced toward helix 3, and away from helix 12, in a manner which moves the N-terminal main chain amide nitrogens into a position too far away to interact with Glu 897 (FIG. 5B).

Comparisons of CRP_{—}3 (FXXLF) and CRP_{—}1 (LXXLL) reveal that the formation of this interaction with Glu 897 is largely dependent on the length of the side chain at the +5 position of the peptide. The shorter Leu at +5 in CRP_{—}1 must reach over to fully make interactions with the hydrophobic +5 binding pocket, effectively pulling the rest of the peptide helix along with it. This causes a displacement of the entire peptide away from helix 12, toward helix 3, and a rotation about Met 734, thereby preventing interaction with Glu 897. On the other hand, the longer Phe residue at +5 of CRP_{—}3 is able to make the full set of interactions at the +5 site without causing a displacement of the peptide helix. Surprisingly though, CRP_{—}5, CRP_{—}4, and CRP_{—}2 instead interact with Glu897 through the hydroxyl groups of their Ser residues at −2.

Unexpected polar interactions were also observed involving Trp residues at +1 and +5. In particular, the structure of CRP_{—}4 (which contains the motif FXXLW in place of LXXLL or FXXLF) bound to AR reveals more information about the coactivator binding pocket, due to the tryptophane residue in the +5 position. In the structure of AR-CRP_{—}4, the indole nitrogen of Trp+5 hydrogen bonds with Gln 733. Specifically, the tryptophane has a charged hydrophylic nitrogen on the indole ring of its side-chain and this ring inserts in the pocket where the plenylalanine in the +5 position of FXXLF would sit. The critical Gln 733 residue of hAR mates with that ring, thereby making a very tight interaction. It is also likely that tyrosine could go in the same place. While it might be expected that W at +5 would be long enough to prevent rotation of the peptide helix and allow main-chain interactions with Glu 897, the CRP_{—}4 (FXXLW) complex reveals that this is not the case. In fact, in order to accommodate the bulky Trp side chain, as well as to form a hydrogen bond between the Trp indole nitrogen and Gln 733, CRP_{—}4 is displaced in a manner such that its binding mode is closer to that of CRP_{—}1 (LXXLL) than CRP_{—}3 (FXXLF). This is important because there is a tryptophane in natural sequences that are thought to bind to the human androgen receptor. The ramifications for designing a small molecule inhibitor of coactivator binding to AR are that such a molecule would not only preferentially have a group that sits in a hydrophobic pocket, but would also have a group that makes a hydrogen bond with Gln 733. Similar interactions are seen in the structure of AR-CRP_{—}2, but in the +1 pocket, where Trp+1 hydrogen bonds with Gln 738.

Although Glu893 is shown in FIGS. 11-15 for all coactivator peptides, it hydrogen bonds only with the main chain amide nitrogen of the −1 residue in CRP_{—}5 and CRP_{—}2. In all other structures it does not make any significant interactions with the coactivator. The side chain of the −1 residue, which is closest to Glu893, is largely disordered in all structures except CRP_{—}3 (FXXLF).

Accordingly, the interactions between the coactivator-related peptides CRP_{—}1, CRP_{—}2, CRP_{—}3, CRP_{—}4 and CRP_{—}5 and various receptor residues can be summarized as follows.

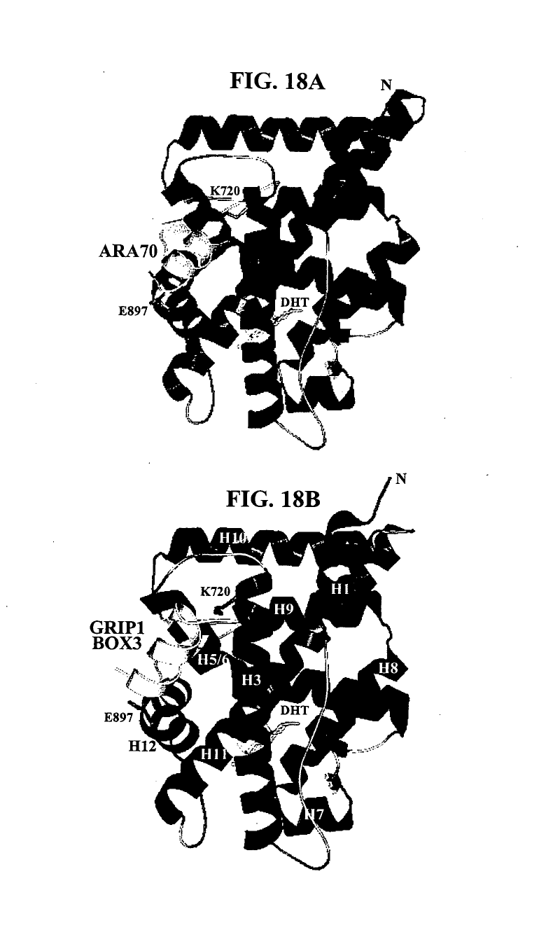

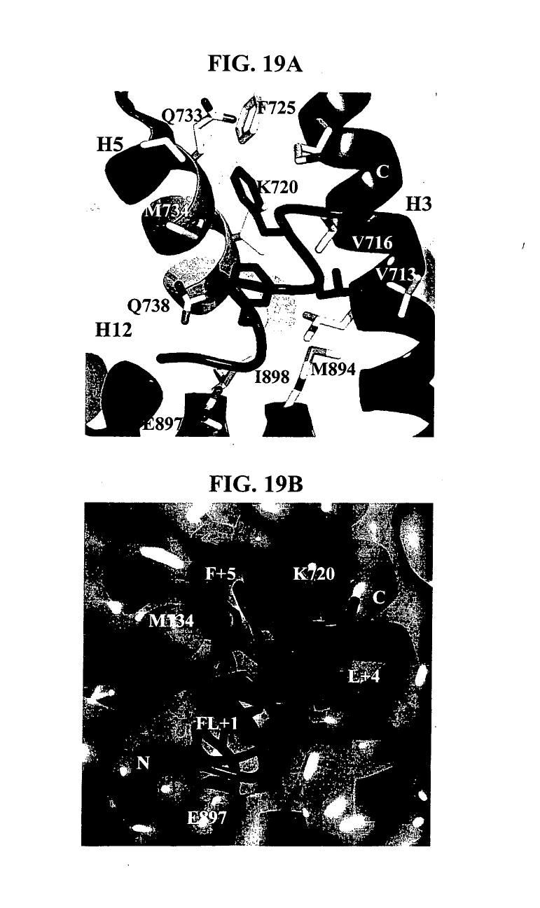

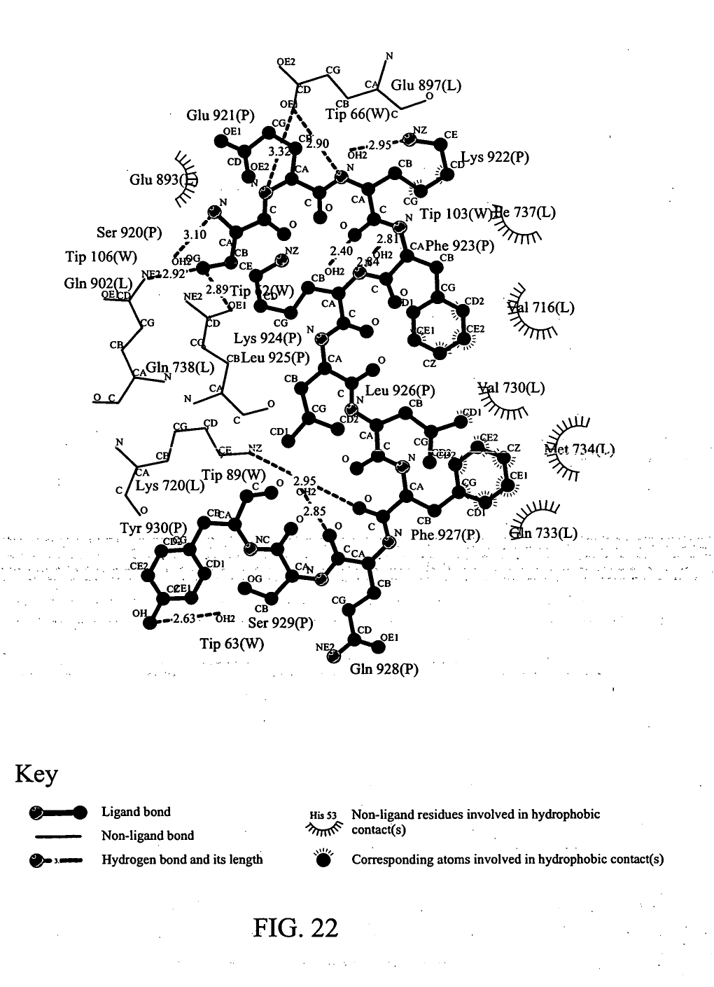

In general, binding to the AR coactivator binding surface is driven primarily by hydrophobic interactions with the amino acid residues at +1 and +5. The key AR coactivator binding site residues Lys 720, Gln 733, Met 734, Gln 738, Met 894, and Glu 897 are shown in each of FIGS. 18-21; in FIG. 9, these residues, with the exception of Gln 733, are also shown. This suggests that Gln 733 is unable to interact with the peptide that contains the motif LXXLL, confirming that this motif does not fully exploit interactions within the +5 binding pocket. In three of the other complexes, Gln 733 makes hydrophobic interactions with the coactivator peptide (FIGS. 18, 19 and 21); in one complex however, Gln733 is able to hydrogen bond with the indole nitrogen on the side chain of the Trp residue in the +5 position of the FXXLW motif (FIG. 22), as further discussed hereinbelow.

Other residues are also shown in FIGS. 9 and 18-21 as making close contacts with coactivator peptides. Val 713 forms part of a shallow cleft that accommodates a residue in the +4 position of the hydrophobic motif. However, Val 713 only interacts with the LXXLL motif (FIG. 9), thus suggesting that LXXLL binding to the AR coactivator binding site is of a different character from that of the other motifs considered herein.

Val 716, which plays a role in three binding clefts, for +1, +4, and +5 residues respectively, forms hydrophobic interactions with all coactivator peptides.

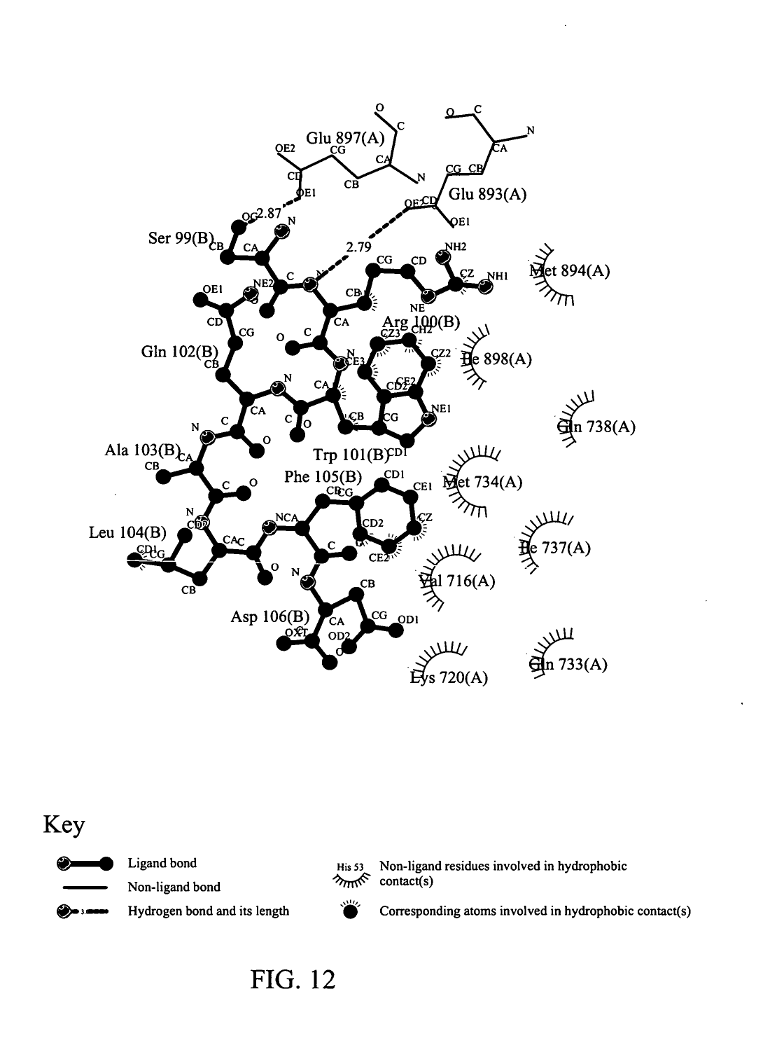

Val 730, which defines part of the +5 binding pocket forms hydrophobic interactions with all of the coactivator peptides considered except those that contain the LXXLL and WXXLF motifs. This is consistent with the interpretation that the leucines in the LXXLL motif reach all the way into the +5 binding pocket but are not long enough to reach Val 730, and also with the interpretation that a hydrogen bond formation between the indole nitrogen of the W+1 residue and the +1 binding pocket prevents the motif from fully exploring the +5 binding pocket. (No H-bond is shown in the LIGPLOT of FIG. 12 because, although the indoie nitrogen is 3.3 Å from the carbonyl group of Gln738, the angle between the groups is such that the program's default rules do not recognize it as an H-bond.)

Ile 737, which defines part of the +5 binding pocket forms hydrophobic interactions with all of the coactivator peptides considered except the one that contains the LXXLL motif. This is consistent with the interpretation that the leucines in the LXXLL motif are not bulky enough to reach all the way into the +5 binding pocket.

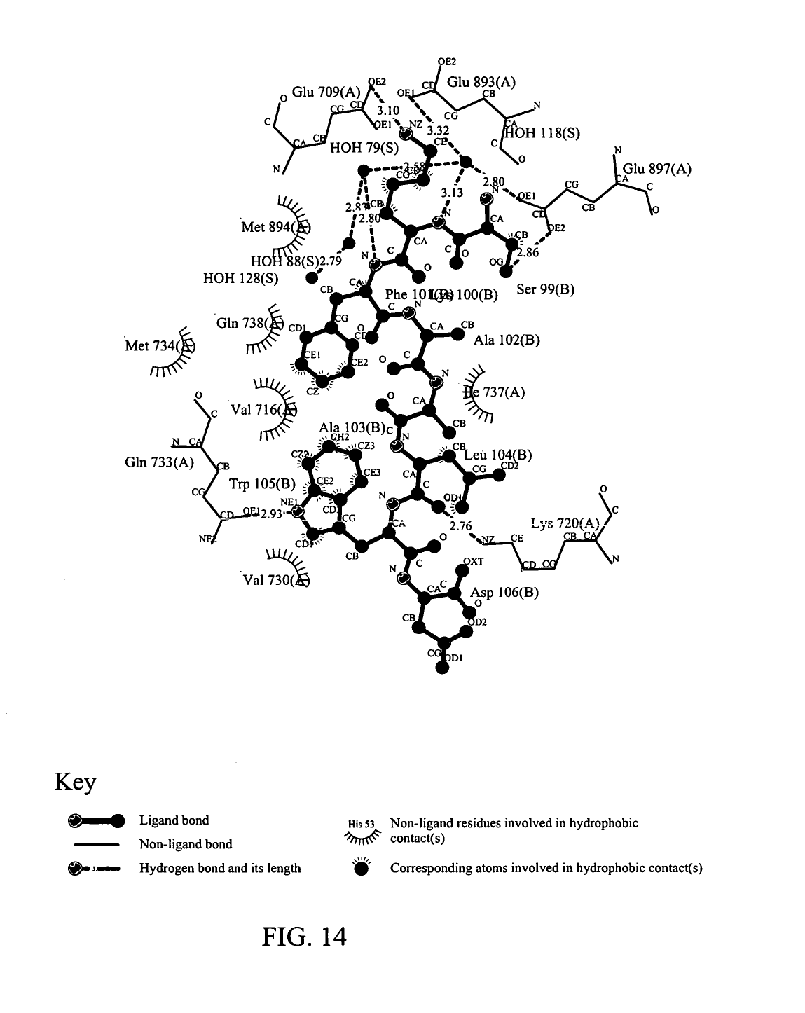

Although Glu 893 does not define any of the binding pockets, it forms an interaction with all coactivator peptides. Although this interaction is hydrophobic with peptides containing the motifs LXXLL, FXXLF, and FXXFF, the Glu 893 residue hydrogen bonds with the Ser-1 residue in peptide containing the motifs FXXLW (FIG. 14), and WXXLF (FIG. 12).

Ile 898, which also does not define any of the binding pockets, only interacts significantly with a coactivator peptide containing the WXXLF motif (FIG. 12).

### Example 7

Structures of AR LBD Bound to Coactivator-Derived Peptides, and the AR:coactivator Interface

The structures of AR LBD bound to coactivator-derived peptides from ARA70 (containing the FXXLF motif), and GRIP1 box3 (containing the LXXLL motif) were obtained. Results presented herein show that both hydrophobic motifs bind in a manner similar to that previously found in other nuclear receptors that bind the LXXLL p160 coactivator motif. Accordingly, the core hydrophobic motif forms a short α-helix that binds in the groove formed by helices 3, 4, 5, and 12. FIGS. 18-21 illustrate, for purposes of comparison, 3 dimensional drawings of the interactions between the AR coactivator binding site and coactivator peptides derived from the GRIP1 box3, and ARA70 coactivators.

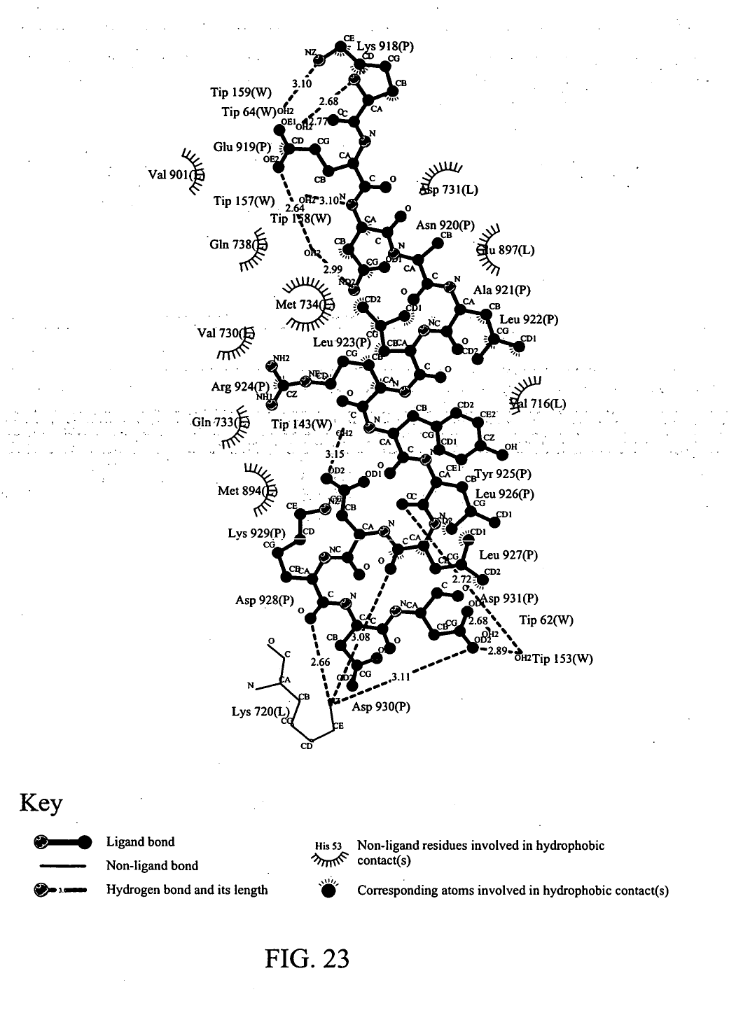

FIGS. 22 and 23 illustrate the binding interactions for the ARA70-derived coactivator peptide (a 15-mer with sequence RETSEKFKLLFQSYN) and the Box3-derived coactivator peptide (a 14-mer with sequence KENALLRYLLDKDD), respectively. The various interactions can be summarized as follows.

The key AR coactivator binding site residues Lys 720, Gln 733, Met 734, Gln 738, and Glu 897 are shown in FIG. 22 for the ARA70-derived peptide containing the motif FXXLF; in FIG. 23, these residues, with the addition of Met 894, are also shown for the Box3-derived peptide. In general, the LXXLL motif does not fully exploit interactions within the +5 binding pocket. Gln 733 makes hydrophobic interactions with both the ARA70-derived coactivator peptide (FIG. 23) and the Box3-derived peptide. The observations for the coactivator-derived peptides are generally consistent with those found for coativator-related peptides, described herein.

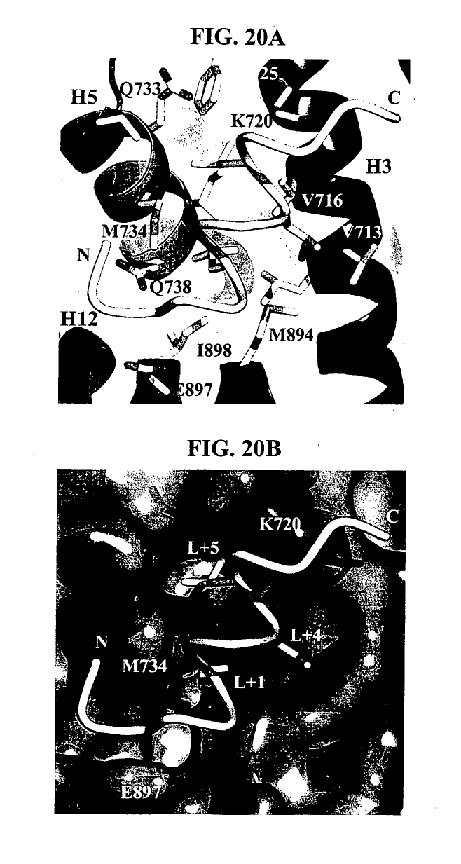

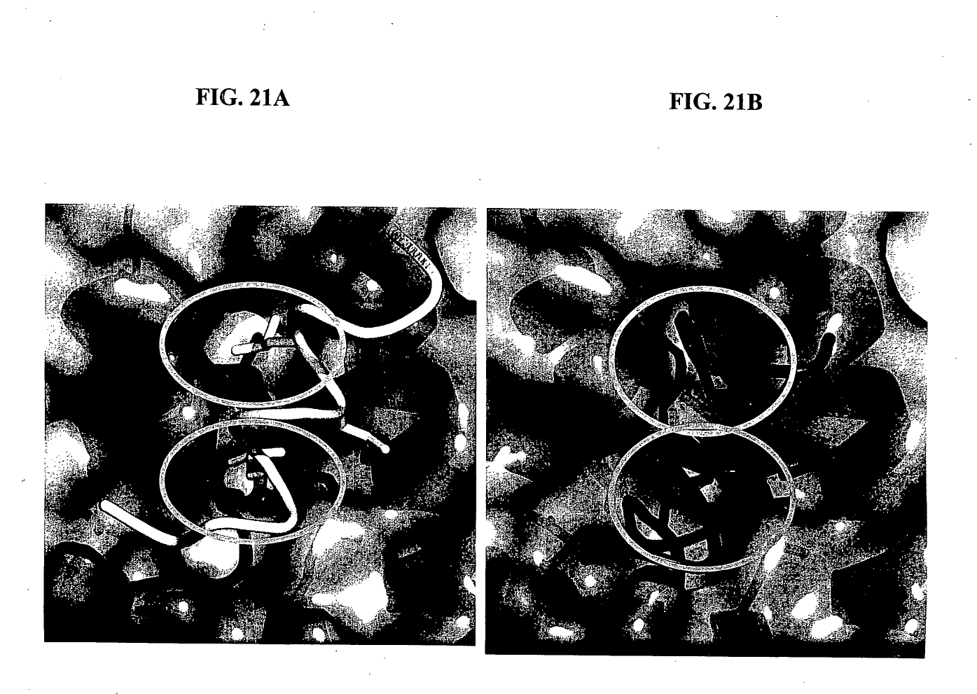

Other residues are also shown in FIGS. 20 and 21 as making close contacts with coactivator peptides.

Residue Val 713 is not seen in either the ARA70-derived or the Box3-derived coactivator peptide interactions with the binding site.

Val 716, which plays a role in three binding clefts, for +1, +4, and +5 residues respectively, forms hydrophobic interactions with both of the coactivator-derived peptides.

Val 730, which defines part of the +5 binding pocket, forms hydrophobic interactions with both the Box3-derived coactivator peptides and the ARA70-derived peptide. In each case, the leucines of the LXXLL motif are able to reach all the way into the +5 binding pocket. One explanation for this is that neither peptide has a side-chain in the +1 position that is able to hydrogen bond with residues in the +1 pocket.

Ile 737, which defines part of the +5 binding pocket forms hydrophobic interactions with the ARA70-derived coactivator peptides but not the Box3-derived peptide, containing the LXXLL motif. This is consistent with the interpretation that the leucines in the LXXLL motif are not bulky enough to reach all the way into the +5 binding pocket.

Although Glu 893 does not define any of the binding pockets, it forms an interaction with both of the coactivator-derived peptides. Just as with the coactivator-related peptides, this interaction is hydrophobic with peptides containing the motifs LXXLL and FXXLF, such as the Box3-derived, and ARA70-derived peptides, respectively.

In terms of polar interactions, both coactivator-derived peptides interact with Lys720, which is one of the conserved charge clamp residues, through main chain carbonyl groups. However, only the ARA70-derived peptide makes a hydrogen-bond with the second charge clamp residue, i.e., Glu897. This observation is consistent with the crystal structure of a coactivator-related peptide containing the FXXLF motif, described hereinabove.

Finally, the ARA70-derived peptide makes an H-bond with Gln738 through its Ser−1 residue, whereas the Box3-derived peptide does not.

Accordingly, overall, the ARA70-derived coactivator peptide containing the FXXLF motif is able to make a greater number of interactions with residues in the AR coactivator binding site than is the Box3-derived peptide containing the LXXLL motif.

Identification and characterization of key residues within ligand binding domain of the AR and extension of this information to other nuclear receptors shows that these residues are common for all nuclear receptors identified to date. Thus, the Examples presented herein demonstrate that information derived from the structure and function of the AR ligand binding domain can be applied in design and selection of compounds that modulate binding of compounds to nuclear receptors for all members of the nuclear receptor family.

### Example 8

Validity of GRIP1 Peptides

It is generally understood that a peptide that contains an NR box motif and flanking residues, as found in naturally occurring GRIP1, will bind to a nuclear receptor in a manner similar to GRIP1 itself. The experiment described in this example demonstrates this in the case of an NR-Box2 peptide and ERα.

GRIP1, a mouse p160 coactivator, interacts both in vivo and in vitro with the ERα LBD bound to agonist (Ding, et al., Mol. Endocrinol, 12:302-313, (1998)), but not with the LBD bound to antagonist (Norris, et al., J. Biol. Chem., 273:6679-88, (1998)). Mutational studies of GRIP1 and its human homologue TIF2 suggest that, of the three NR boxes from GRIP1, NR box 2 binds most tightly to the ERα LBD (Ding, et al., Mol. Endocrinol, 12:302-313, (1998), and Voegel, et al., EMBO J., 15:3667-3675 (1996)).

Competition assays indicate that a 13 residue GRIP1 NR Box 2 peptide, NH₂-KHKILHRLLQDSS-CO₂H (SEQ ID NO: 25), (Ding, et al., Mol. Endocrinol, 12:302-313, (1998)), synthesized by standard solid phase methods, binds specifically to the agonist-bound ERα LBD (IC50<0.4 μM) and to other agonist-bound NR LBD's (Ding, et al., Mol. Endocrinol, 12:302-313, (1998), and Darimont, et al., Genes Dev., 1,12(21), 3343-56, (1998)).

An electrophoretic mobility shift assay was used to demonstrate that the GRIP1 NR Box 2 peptide (SEQ ID NO:4) bound the ERα LBD in the presence of the agonist, diethylstilbestrol (DES), but not the antagonist, OHT. Eight microgram samples of purified hERα-LBD bound to either DES or OHT were incubated in the absence of the GRIP1 NR Box 2 peptide (SEQ ID NO:4), i.e., buffer alone, or in the presence of either a 2-fold or 10-fold molar excess of the GRIP1 NR Box 2 peptide. The binding reactions were performed on ice for 45 minutes in 10 μl of buffer containing 20 mM Tris, pH 8.1, 1 mM DTT, and 200 mM NaCl and then subjected to 6% native PAGE. Gels were stained with GELCODE Blue Stain reagent (Pierce).

In the presence of the NR box 2 peptide, the migration of the DES-LBD complex was retarded. In contrast, peptide addition had no effect on the mobility of the OHT-LBD complex. Hence, this peptide fragment of GRIP1 possesses the ligand-dependent receptor binding activity characteristic of the full-length protein. These observations suggest that the GRIP1 NR Box 2 peptide is a valid model for studying the interaction between GRIP1 and the ERα LBD, and further suggest that peptides containing GRIP1 nuclear receptor box sequences represent appropriate mimics of GRIP1 binding to nuclear receptors.

### Example 9

Design of Coactivator Inhibitors

Using an atomic structure of the AR coactivator binding site of the present invention, a coactivator binding motif such as FXXLW (as found in the peptide CRP_{—}4), was placed into the coactivator binding site using the computer modeling program, Insight, available from Accelrys corporation. From such a model, it was deduced that the indole ring on the side chain of the tryptophan (W) residue of the peptide motif WXXLF fit into a first binding pocket on the AR coactivator binding site, and that the phenyl ring of phenylalanine (F) residue of the peptide motif FXXLF fit into a second binding pocket on the AR coactivator binding site.

Using the program LUDI (available from Accelrys corporation) an indole ring system and a benzene ring were placed into the binding site pocket filled respectively with the side-chain residues of tryptophan and phenylalanine. This can be carried out by “deleting” the remaining atoms of the FXXLW motif of a model of the CRP_{—}4 peptide when placed in the coactivator binding site in its receptor-bound conformation, as found in the crystal structures of the present invention.

The program LUDI then supplies a selection of “linkers”, i.e., sequences of functional groups that can bridge between the indole and phenyl rings without clashing with other receptor atoms.

The fact that the two aromatic rings can fit closely into binding pockets on the coactivator binding site means that a viable coactivator binder can be designed that only utilizes two attachment points on the receptor surface. Sufficient binding energy can be obtained from two points only whereas an inhibitor that mimics the interactions of, say, the motif LXXLL would require 3 attachment points, one corresponding to each of the three leucine residues. Furthermore, such an inhibitor would require a rather strained scaffold in order to fit into the coactivator binding site as well as maintaining the three points of attachment.

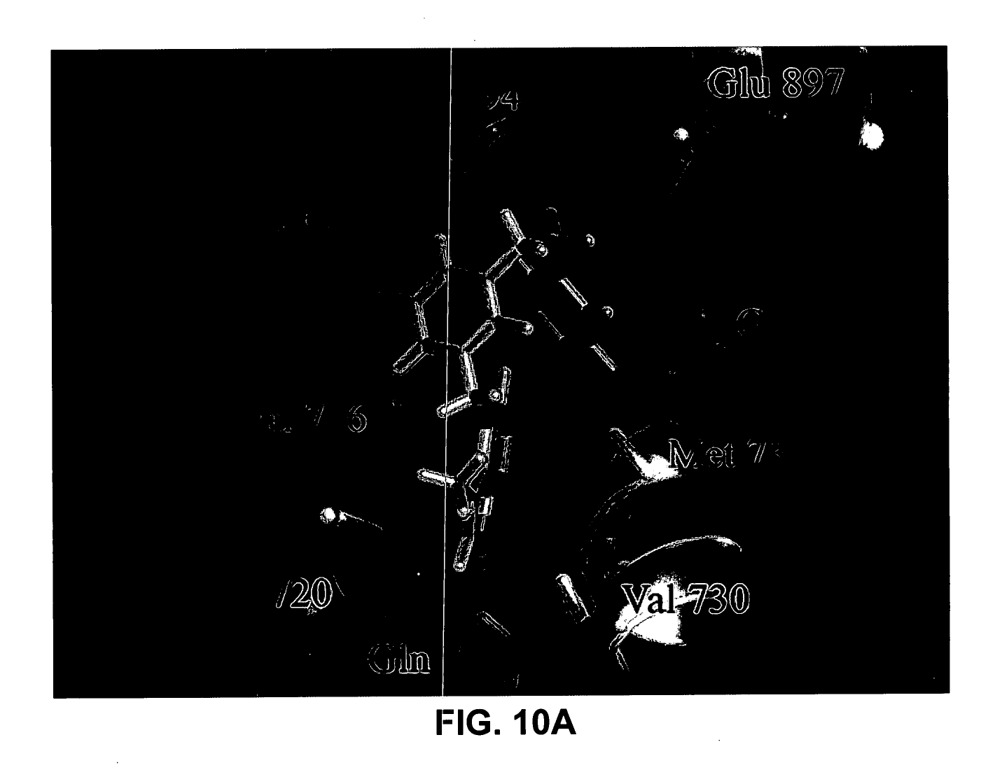

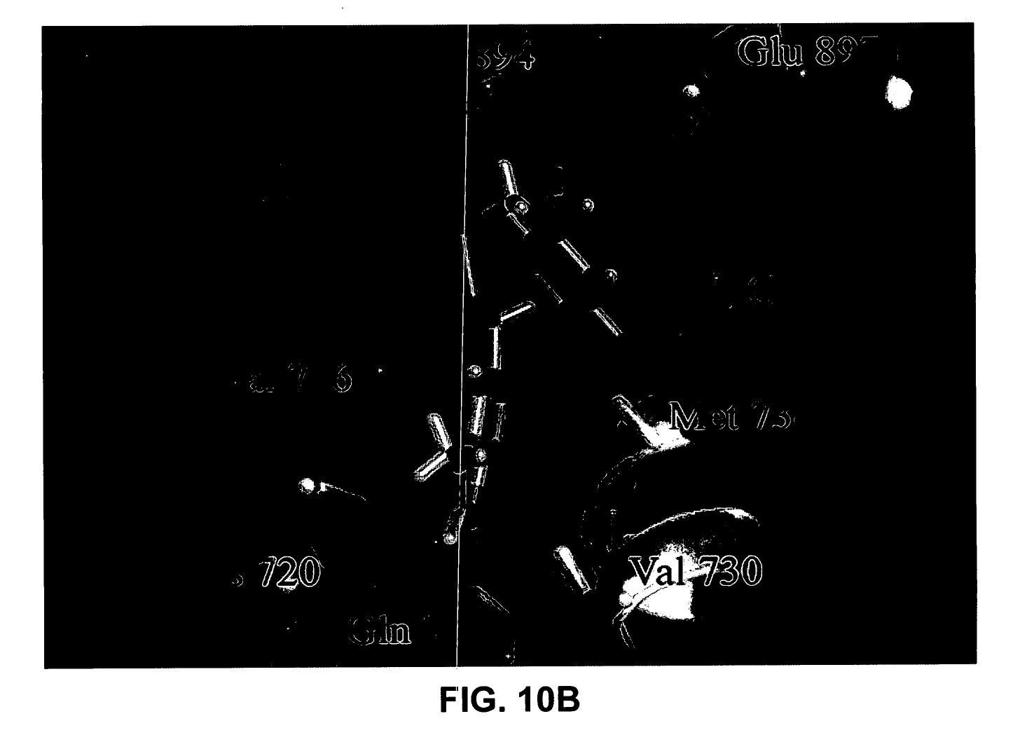

Accordingly, molecules (I) and (II), presented hereinabove, have been designed and proposed to be coactivator inhibitors of AR. FIGS. 10A and 10B show, respectively, conformations of molecules (I) and (II) docked into an atomic structural model of the AR coactivator binding site, taken from the structure of CRP_{—}4 (FXXLW) CRP_{—}4 bound to AR LBD, see Appendix 4Z. The indole ring of each molecule is in close contact with residue Val 730, shown in each figure.

The program LUDI can provide a binding score, the “LUDI score”, which is an empirical measure of how well a proposed structure fits into the binding site, and which can be related to a binding constant, K_{d} for the structure in question. LUDI scores for molecules (I) and (II), and for the peptide CRP_{—}4, are presented in Table 4.

TABLE 4

| Molecule | LUDI Score | Kᵢ |
|---|---|---|
| Peptide CRP_4 | 189 | 10 mM |
| Molecule 1 | 422 | 90 μM |
| Molecule 2 | 361 | 500 μM |

### Appendices

Each Protein Data Bank (PDB) file, presented in Tables 1 and 2, found respectively in the files identified as Table1_ARLBD_DHT_CDP.txt and Table2_ARLBD_DHT_CRP.txt, presented on CD-R herewith, contains coordinates of at least one protein fragment. As would be understood by one of skill in the art, a PDB file provides a sequence of amino acids in order of connectivity (primary sequence) in one or more polypeptide chains. The format of a PDB file is well known in the art, and a description is available on the world wide web at www.rcsb.org/pdb/docs/format/pdbguide2.2/guide2.2_frame.html. This description demonstrates that, in particular, a PDB file contains the atomic coordinates, residue name, and sequence number of each resolved non-hydrogen atom in a crystal structure of a protein, protein complex, or protein fragment. Where there is more than one polypeptide chain, a terminator (“TER”) can be indicated to separate the chains, or a gap in residue sequence numbering can be used. One of ordinary skill in the art would be able to deduce an amino acid sequence of a protein or polypeptide directly from a PDB file, with no additional information. Accordingly, the PDB files presented in the instant specification provides a description for each amino acid sequence listed.

The sequence numbering of the AR LBD is the same as that in the wild-type human form of AR with SwissProt accession number P10275, found at us.expasy.org/cgi-bin/niceprot.p1?P10275. Although, the construct used to make the crystal structures presented herein is from chimp (see SwissProt O97775), not human, the chimp and human AR are almost exactly the same. There are 6 differences between the human and chimp sequences, comprising 3 substitutions and 3 differences in the length of poly-glycine and poly-glutamine repeats, all of which appear in the NTD; the sequences of the LBD's are exactly the same as one another.

Appendices 1Z-10Z contain headers from the respective PDB files of coordinates in Tables 1 and 2. Specifically, the correspondence is as follows: the headers in Appendices 1Z-8Z correspond, respectively, to the PDB files at (A)-(H) of Table 2; the headers in Appendices 9Z and 10Z correspond, respectively, to the PDB files at (A) and (B) of Table 1. The headers contain data about the crystal structure and the parameters used to obtain the coordinates. The headers are in the normal PDB format for “remarks” that accompany the coordinate data, and the terms used therein are intelligible to one of ordinary skill in the art.

The sequence of the AR LBD is:

(SEQ ID NO: 28)

MEVQLGLGRVYPRPPSKTYRGAFQNLFQSVREVIQNPGPRHPEAASAAPP
GASLLLLQQQQQQQQQQQQQQQQQQQQQETSPRQQQQQQGEDGSPQAHRR
GPTGYLVLDEEQQPSQPQSALECHPERGCVPEPGAAVAASKGLPQQLPAP
PDEDDSAAPSTLSLLGPTFPGLSSCSADLKDILSEASTMQLLQQQQQEAV
SEGSSSGRAREASGAPTSSKDNYLGGTSTISDNAKELCKAVSVSMGLGVE
ALEHLSPGEQLRGDCMYAPLLGVPPAVRPTPCAPLAECKGSLLDDSAGKS
TEDTAEYSPFKGGYTKGLEGESLGCSGSAAAGSSGTLELPSTLSLYKSGA
LDEAAAYQSRDYYNFPLALAGPPPPPPPPHPHARIKLENPLDYGSAWAAA
AAQCRYGDLASLHGAGAAGPGSGSPSAAASSSWHTLFTAEEGQLYGPCGG
GGGGGGGGGGGGGGGGGGGGGGEAGAVAPYGYTRPPQGLAGQESDFTAPD
VWYPGGMVSRVPYPSPTCVKSEMGPWMDSYSGPYGDMRLETARDHVLPID
YYFPPQKTCLICGDEASGCHYGALTCGSCKVFFKRAAEGKQKYLCASRND
CTIDKFRRKNCPSCRLRKCYEAGMTLGARKLKKLGNLKLQEEGEASSTTS
PTEETTQKLTVSHIEGYECQPIFLNVLEAIEPGVVCAGHDNNQPDSFAAL
LSSLNELGERQLVHVVKWAKALPGFRNLHVDDQMAVIQYSWMGLMVFAMG
WRSFTNVNSRMLYFAPDLVFNEYRMHKSRMYSQCVRMRHLSQEFGWLQIT
PQEFLCMKALLLFSIIPVDGLKNQKFFDELRMNYIKELDRIIACKRKNPT
SCSRRFYQLTKLLDSVQPIARELHQFTFDLLIKSHMVSVDFPEMMAEIIS
VQVPKILSGKVKPIYFHTQ.

The portion of AR LBD used in crystallography described herein, and which is present in the PDB files in Tables 1 and 2, typically starts at residue Gln670 and ends at around residue Gln919:

(SEQ ID NO: 29)

QPIFLNVLEAIEPGVVCAGHDNNQPDSFAALLSSLNELGERQLVHVVKW
AKALPGFRNLHVDDQMAVIQYSWMGLMVFAMGWRSFTNVNSRMLYFAPD
LVFNEYRMHKSRMYSQCVRMRHLSQEFGWLQITPQEFLCMKALLLFSII
PVDGLKNQKFFDELRMNYIKELDRIIACKRKNPTSCSRRFYQLTKLLDS
VQPIARELHQFTFDLLIKSHMVSVDFPEMMAEIISVQVPKILSGKVKPI
YFHTQ.

Variants start at residue Cys669 and other variants end at residue Tyr918.
Appendix 1Z

Header of PDB file containing atomic coordinates for human AR complexed with DHT and a coactivator-related peptide, designated CRP_{—}1, containing the motif LXXLL.

REMARK Created by MOLEMAN
REMARK coordinates from restrained individual B-factor refinement
REMARK refinement resolution: 20.0 − 1.6 A
REMARK starting r= 0.2017 free_r= 0.2220
REMARK final r= 0.1989 free_r= 0.2197
REMARK rmsd bonds= 0.005787 rmsd angles= 1.06472
REMARK B rmsd for bonded mainchain atoms= 1.185 target= 1.5
REMARK B rmsd for bonded sidechain atoms= 2.132 target= 2.0
REMARK B rmsd for angle mainchain atoms= 1.810 target= 2.0
REMARK B rmsd for angle sidechain atoms= 3.187 target= 2.5
REMARK rweight= 0.1000 (with wa= 0.794839)
REMARK target= mlf steps= 30
REMARK sg= P2(1)2(1)2(1) a= 54.225 b= 66.327 c= 69.407 alpha= 90 beta= 90
gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK ncs= none
REMARK B-correction resolution: 6.0 − 1.6
REMARK initial B-factor correction applied to fobs:
REMARK B11= −9.002 B22= 5.334 B33= 3.668
REMARK B12= 0.000 B13= 0.000 B23= 0.000
REMARK B-factor correction applied to coordinate array B: −0.051
REMARK bulk solvent: density level= 0.411252 e/A\hat{ }3, B-factor= 53.0348 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 33690 (100.0 %) |
| REMARK number of unobserved reflections (no entry or \|F\|=0): | 953 (2.8 %) |
| REMARK number of reflections rejected: | 0 (0.0 %) |
| REMARK total number of reflections used: | 32737 (97.2 %) |
| REMARK number of reflections in working set: | 31107 (92.3 %) |
| REMARK number of reflections in test set: | 1630 (4.8 %) |
| REMARK VERSION:1.1 | |

| | |
|---|---|
| CRYST1 | 54.225 66.327 69.407 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.018442 0.000000 0.000000 0.00000 |
| SCALE2 | 0.000000 0.015077 0.000000 0.00000 |
| SCALE3 | 0.000000 0.000000 0.014408 0.00000 |

Appendix 2Z

Header of PDB file containing atomic coordinates for human AR complexed with DHT and a coactivator-related peptide, designated CRP_{—}2, containing the motif WXXLF.

REMARK Created by MOLEMAN
REMARK coordinates from minimization refinement
REMARK refinement resolution: 20.0 − 2.2 A
REMARK starting r= 0.2096 free_r= 0.2488
REMARK final r= 0.2083 free_r= 0.2473
REMARK rmsd bonds= 0.007093 rmsd angles= 1.13564
REMARK wa= 2.4098
REMARK target= mlf cycles= 1 steps= 400
REMARK sg= P2(1)2(1)2(1) a= 55.947 b= 66.434 c= 71.720 alpha= 90 beta= 90
gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK parameter file 4 : CNS_TOPPAR:ion.param
REMARK ncs= none
REMARK B-correction resolution: 6.0 − 2.2
REMARK initial B-factor correction applied to fobs :
REMARK B11= −32.766 B22= 18.474 B33= 14.292
REMARK B12= 0.000 B13= 0.000 B23= 0.000
REMARK B-factor correction applied to coordinate array B: −0.532
REMARK bulk solvent: density level= 0.365634 e/A\hat{ }3, B-factor= 54.1419 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 14088 ( 100.0 % ) |
| REMARK number of unobserved reflections (no entry or \|F\|=0): | 741 ( 5.3 % ) |
| REMARK number of reflections rejected: | 0 ( 0.0 % ) |
| REMARK total number of reflections used: | 13347 ( 94.7 % ) |
| REMARK number of reflections in working set: | 12669 ( 89.9 % ) |
| REMARK number of reflections in test set: | 678 ( 4.8 % ) |
| REMARK VERSION:1.1 | |

| | |
|---|---|
| CRYST1 | 55.947 66.434 71.720 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.017874 0.000000 0.000000 0.00000 |
| SCALE2 | 0.000000 0.015053 0.000000 0.00000 |
| SCALE3 | 0.000000 0.000000 0.013943 0.00000 |

Appendix 3Z

Header of PDB file containing atomic coordinates for human AR complexed with DHT and a coactivator-related peptide, designated CRP_{—}3, containing the motif FXXLF.

REMARK Created by MOLEMAN
REMARK coordinates from restrained individual B-factor refinement
REMARK refinement resolution: 20.0 − 1.45 A
REMARK starting r= 0.1954 free_r= 0.2034
REMARK final r= 0.1953 free_r= 0.2033
REMARK rmsd bonds= 0.005800 rmsd angles= 1.09832
REMARK B rmsd for bonded mainchain atoms= 1.101 target= 1.5
REMARK B rmsd for bonded sidechain atoms= 2.209 target= 2.0
REMARK B rmsd for angle mainchain atoms= 1.699 target= 2.0
REMARK B rmsd for angle sidechain atoms= 3.220 target= 2.5
REMARK rweight= 0.1000 (with wa= 0.53362)
REMARK target= mlf steps=30
REMARK sg= P2(1)2(1)2(1) a= 55.407 b= 66.200 c= 68.832 alpha= 90 beta= 90
gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK parameter file 4 : CNS_TOPPAR:ion.param
REMARK ncs= none
REMARK B-correction resolution: 6.0 − 1.45
REMARK initial B-factor correction applied to fobs :
REMARK B11= −9.249 B22= 6.274 B33= 2.975
REMARK B12= 0.000 B13= 0.000 B23= 0.000
REMARK B-factor correction applied to coordinate array B: −0.003
REMARK bulk solvent: density level= 0.395754 e/A\hat{ }3, B-factor= 50.2566 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 45540 ( 100.0 % ) |
| REMARK number of unobserved reflections (no entry or \|F\|=0): | 287 ( 0.6 % ) |
| REMARK number of reflections rejected: | 0 ( 0.0 % ) |
| REMARK total number of reflections used: | 45253 ( 99.4 % ) |
| REMARK number of reflections in working set: | 43006 ( 94.4 % ) |
| REMARK number of reflections in test set: | 2247 ( 4.9 % ) |
| REMARK VERSION: 1.1 | |

| | |
|---|---|
| CRYST1 | 55.407 66.200 68.832 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.018048 0.000000 0.000000 0.00000 |
| SCALE2 | 0.000000 0.015106 0.000000 0.00000 |
| SCALE3 | 0.000000 0.000000 0.014528 0.00000 |

Appendix 4Z

Header of PDB file containing atomic coordinates for human AR complexed with DHT and a coactivator-related peptide, designated CRP_{—}4, containing the motif FXXLW.

REMARK Created by MOLEMAN
REMARK from bind0908.pdb
REMARK coordinates from restrained individual B-factor refinement
REMARK refinement resolution: 20.0 − 1.8 A
REMARK starting r= 0.2016 free_r= 0.2380
REMARK final r= 0.2007 free_r= 0.2350
REMARK rmsd bonds= 0.006350 rmsd angles= 0 1.05669
REMARK B rmsd for bonded mainchain atoms= 1.265 target= 1.5
REMARK B rmsd for bonded sidechain atoms= 2.139 target= 2.0
REMARK B rmsd for angle mainchain atoms= 1.853 target= 2.0
REMARK B rmsd for angle sidechain atoms= 3.264 target= 2.5
REMARK rweight= 0.1000 (with wa= 1.28071)
REMARK target= mlf steps= 30
REMARK sg= P2(1)2(1)2(1) a= 55.826 b= 66.128 c= 68.381 alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK ncs= none
REMARK B-correction resolution: 6.0 − 1.8
REMARK initial B-factor correction applied to fobs :
REMARK B11= −14.446 B22= 7.925 B33= 6.521
REMARK B12= 0.000 B13= 0.000 B23= 0.000
REMARK B-factor correction applied to coordinate array B: 0.051
REMARK bulk solvent: density level= 0.373424 e/A\hat{ }3, B-factor = 50.5977 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 24048 ( 100.0 % ) |
| REMARK number of unobserved reflections (no entry or \|F\|=0): | 277 ( 1.2 % ) |
| REMARK number of reflections rejected: | 0 ( 0.0 % ) |
| REMARK total number of reflections used: | 23771 ( 98.8 % ) |
| REMARK number of reflections in working set: | 22619 ( 94.1 % ) |
| REMARK number of reflections in test set: | 1152 ( 4.8 % ) |
| REMARK VERSION: 1.1 | |

| | |
|---|---|
| CRYST1 | 55.826 66.128 68.381 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.017913 0.000000 0.000000 0.00000 |
| SCALE2 | 0.000000 0.015122 0.000000 0.00000 |
| SCALE3 | 0.000000 0.000000 0.014624 0.00000 |

Appendix 5Z

Header of PDB file containing atomic coordinates for human AR complexed with DHT and a coactivator-related peptide, designated CRP_, containing the motif FXXFF.

REMARK Created by MOLEMAN
REMARK coordinates from restrained individual B-factor refinement
REMARK refinement resolution: 20.0 − 2.2 A
REMARK starting r= 0.1996 free_r= 0.2471
REMARK final r= 0.1994 free_r= 0.2464
REMARK rmsd bonds= 0.006854 rmsd angles= 1.05874
REMARK B rmsd for bonded mainchain atoms= 1.445 target= 1.5
REMARK B rmsd for bonded sidechain atoms= 2.249 target= 2.0
REMARK B rmsd for angle mainchain atoms= 2.355 target= 2.0
REMARK B rmsd for angle sidechain atoms= 3.242 target= 2.5
REMARK rweight= 0.1000 (with wa= 2.52582)
REMARK target= mlf steps= 30
REMARK sg= P2(1)2(1)2(1) a= 54.323 b= 66.592 c= 70.223
REMARK alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK parameter file 4 : CNS_TOPPAR:ion.param
REMARK ncs= none
REMARK B-correction resolution: 6.0 − 2.2
REMARK initial B-factor correction applied to fobs :
REMARK B11= −14.285 B22= 6.754 B33= 7.531
REMARK B12= 0.000 B13= 0.000 B23= 0.000
REMARK B-factor correction applied to coordinate array B: −0.076
REMARK bulk solvent: density level= 0.367359 e/A\hat{ }3, B-factor= 47.4855 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 13432 (100.0 %) |
| REMARK number of unobserved reflections (no entry or \|F\|=0): | 100 ( 0.7 %) |
| REMARK number of reflections rejected: | 0 ( 0.0 %) |
| REMARK total number of reflections used: | 13332 ( 99.3 %) |
| REMARK number of reflections in working set: | 12653 ( 94.2 %) |
| REMARK number of reflections in test set: | 679 ( 5.1 %) |
| REMARK VERSION: 1.1 | |

| | |
|---|---|
| CRYST1 | 54.323 66.592 70.223 90.00 90.00 90.00 P 21 21 2110 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.018408 0.000000 0.000000 0.00000 |
| SCALE2 | 0.000000 0.015017 0.000000 0.00000 |
| SCALE3 | 0.000000 0.000000 0.014240 0.00000 |

Appendix 6Z

Header of PDB file containing atomic coordinates for human AR complexed with DHT and a coactivator-related peptide, designated CRP_{—}6, containing the motif FXXYF.

REMARK Created by MOLEMAN
REMARK MoleMan PDB file
REMARK coordinates from restrained individual B-factor refinement
REMARK refinement resolution: 20.0 − 1.6 A
REMARK starting r= 0.2134 free_r= 0.2256
REMARK final r= 0.1996 free_r= 0.2103
REMARK rmsd bonds= 0.005552 rmsd angles= 1.04355
REMARK B rmsd for bonded mainchain atoms= 1.184 target= 1.5
REMARK B rmsd for bonded sidechain atoms= 2.079 target= 2.0
REMARK B rmsd for angle mainchain atoms= 1.897 target= 2.0
REMARK B rmsd for angle sidechain atoms= 3.124 target= 2.5
REMARK rweight= 0.1000 (with wa= 0.709632)
REMARK target= mlf steps= 30
REMARK sg= P2(1)2(1)2(1) a= 55.591 b= 66.641 c= 72.484
REMARK alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK parameter file 4 : CNS_TOPPAR:ion.param
REMARK ncs= none
REMARK B-correction resolution: 6.0 − 1.6
REMARK initial B-factor correction applied to fobs :
REMARK B11= −4.748 B22= 5.274 B33= −0.525
REMARK B12= 0.000 B13= 0.000 B23= 0.000
REMARK B-factor correction applied to coordinate array B: −0.001
REMARK bulk solvent: density level= 0.393463 e/A\hat{ }3, B-factor= 45.2598 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 36193 (100.0 %) |
| REMARK number of unobserved reflections (no entry or \|F\|=0): | 3 (0.0 % ) |
| REMARK number of reflections rejected: | 0 (0.0 % ) |
| REMARK total number of reflections used: | 36190 (100.0 %) |
| REMARK number of reflections in working set: | 34396 (95.0 % ) |
| REMARK number of reflections in test set: | 1794 (5.0 % ) |
| REMARK VERSION: 1.1 | |

| | |
|---|---|
| CRYST1 | 55.591 66.641 72.484 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.017989 0.000000 0.000000 0.00000 |
| SCALE2 | 0.000000 0.015006 0.000000 0.00000 |
| SCALE3 | 0.000000 0.000000 0.013796 0.00000 |

Appendix 7Z

Header of PDB file containing atomic coordinates for human AR complexed with DHT and a coactivator-related peptide, designated CRP_{—}7, containing the motif WXXVW.

REMARK Created by MOLEMAN
REMARK from bind1105.pdb
REMARK Lys845,847 trimmed
REMARK coordinates from restrained individual B-factor refinement
REMARK refinement resolution: 20.0 − 2.1 A
REMARK starting r= 0.2093 free_r= 0.2432
REMARK final r= 0.2093 free_r= 0.2437
REMARK rmsd bonds= 0.006412 rmsd angles= 1.07635
REMARK B rmsd for bonded mainchain atoms= 1.395 target= 1.5
REMARK B rmsd for bonded sidechain atoms= 2.217 target= 2.0
REMARK B rmsd for angle mainchain atoms= 2.222 target= 2.0
REMARK B rmsd for angle sidechain atoms= 3.205 target= 2.5
REMARK rweight= 0.1000 (with wa= 2.12555)
REMARK target= mlf steps= 50
REMARK sg= P2(1)2(1)2(1) a= 53.386 b= 66.420 c= 70.606
REMARK alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK parameter file 4 : CNS_TOPPAR:ion.param
REMARK ncs= none
REMARK B-correction resolution: 6.0 − 2.1
REMARK initial B-factor correction applied to fobs :
REMARK B11= −19.219 B22= 11.420 B33= 7.798
REMARK B12= 0.000 B13= 0.000 B23= 0.000
REMARK B-factor correction applied to coordinate array B: 0.009
REMARK bulk solvent: density level= 0.366511 e/A\hat{ }3, B-factor= 54.8765 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 15185 (100.0 % ) |
| REMARK number of unobserved reflections (no entry or \|F\|=0): | 15 ( 0.1 % ) |
| REMARK number of reflections rejected: | 0 ( 0.0 % ) |
| REMARK total number of reflections used: | 15170 ( 99.9 % ) |
| REMARK number of reflections in working set: | 14417 ( 94.9 % ) |
| REMARK number of reflections in test set: | 753 ( 5.0 % ) |
| REMARK VERSION: 1.1 | |

| | |
|---|---|
| CRYST1 | 53.386 66.420 70.606 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.018732 0.000000 0.000000 0.00000 |
| SCALE2 | 0.000000 0.015056 0.000000 0.00000 |
| SCALE3 | 0.000000 0.000000 0.014163 0.00000 |

coactivator binding site to be deduced. As described hereinabove, many coactivators recognize agonist bound nuclear receptor LBD's through the sequence motif LXXLL (SEQ ID [[NO: 1),]] NO: 2) where L is leucine and X is any amino acid, a motif which is also referred to as the nuclear receptor box (“NR-box”). The LXXLL motif (SEQ ID [[NO: 1),]] NO: 2) forms the core of a short amphipathic α-helix which is recognized by a highly complementary hydrophobic groove on the surface of the nuclear receptor. This peptide binding groove is the coactivator binding site and is formed by residues from helices 3, 4, 5 and 12 and the turn between helices 3 and 4. The groove lies on the surface of a nuclear receptor ligand binding domain. The floor and sides of this groove are completely nonpolar, but the ends of this groove are charged. These features have also been seen in the structure of the DES-hERα LBD-GRIP1 peptide complex. Furthermore, structural studies of the complex between TRβ and the GRIP1 NR box 2 peptide, biochemical studies of GRIP1 binding to TRβ and GR (Darimont, et al., Genes Dev., 12:3343-3356, (1998)), and a study of the general features of the PPARγ/SRC-1 peptide complex (Nolte, et al., Nature, 395:137-143, (1998)) suggest that certain mechanisms of NR box recognition are probably conserved across the nuclear receptor family. Nevertheless, differences between the coactivator binding sites, and ligand binding domains, of various nuclear receptors have emerged, and a definitive understanding of the structure of a given coactivator binding site is facilitated by having access to a crystal structure thereof, particularly one comprising a bound coactivator.
Appendix 8Z

Header of PDB file containing atomic coordinates for human AR complexed with DHT and a coactivator-related peptide, designated CRP_{—}8, containing the motif FXXLF.

REMARK Created by MOLEMAN
REMARK MoleMan PDB file
REMARK Lys845,847 trimmed
REMARK residues flanking FxxLF left as alanine
REMARK coordinates from restrained individual B-factor refinement
REMARK refinement resolution: 20.0 − 1.9 A
REMARK starting r= 0.2224 free_r= 0.2473
REMARK final r= 0.2110 free_r= 0.2421
REMARK rmsd bonds= 0.006394 rmsd angles= 1.04902
REMARK B rmsd for bonded mainchain atoms= 1.418 target= 1.5
REMARK B rmsd for bonded sidechain atoms= 2.179 target= 2.0
REMARK B rmsd for angle mainchain atoms= 2.218 target= 2.0
REMARK B rmsd for angle sidechain atoms= 3.266 target= 2.5
REMARK rweight= 0.1000 (with wa= 1.59283)
REMARK target= mlf steps= 30
REMARK sg= P2(1)2(1)2(1) a= 54.221 b= 66.261 c= 70.259
REMARK alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : /home3/rms/ehur/ar/cns/dht_ligand.par
REMARK parameter file 4 : CNS_TOPPAR:ion.param
REMARK ncs= none
REMARK B-correction resolution: 6.0 − 1.9
REMARK initial B-factor correction applied to fobs :
REMARK B11= −15.447 B22= 10.673 B33= 4.774
REMARK B12= 0.000 B13= 0.000 B23= 0.000
REMARK B-factor correction applied to coordinate array B: −0.151
REMARK bulk solvent: density level= 0.361298 e/A\hat{ }3, B-factor= 53.2441 A\hat{ }2
REMARK reflections with |Fobs|/sigma_F > 0.0 rejected
REMARK reflections with |Fobs| > 10000 * rms(Fobs) rejected

| | |
|---|---|
| REMARK theoretical total number of refl. in resol. range: | 20528 (100.0 % ) |
| REMARK number of unobserved reflections (no entry or \|F\|=0): | 23 ( 10.1 % ) |
| REMARK number of reflections rejected: | 0 ( 0.0 % ) |
| REMARK total number of reflections used: | 20505 ( 99.9 % ) |
| REMARK number of reflections in working set: | 19512 ( 95.1 % ) |
| REMARK number of reflections in test set: | 993 ( 4.8 % ) |
| REMARK VERSION: 1.1 | |

| | |
|---|---|
| CRYST1 | 54.221 66.261 70.259 90.00 90.00 90.00 P 21 21 21 1 |
| ORIGX1 | 1.000000 0.000000 0.000000 0.00000 |
| ORIGX2 | 0.000000 1.000000 0.000000 0.00000 |
| ORIGX3 | 0.000000 0.000000 1.000000 0.00000 |
| SCALE1 | 0.018443 0.000000 0.000000 0.00000 |
| SCALE2 | 0.000000 0.015092 0.000000 0.00000 |
| SCALE3 | 0.000000 0.000000 0.014233 0.00000 |

Appendix 9Z

Header of PDB file containing atomic Coordinates for human AR LBD complexed with DHT and an ARA70-derived Peptide, designated CDP_{—}1, containing the FXXLF motif.

REMARK coordinates from minimization and B-factor refinement
REMARK refinement resolution: 30.0 − 2.3 A
REMARK starting r= 0.2250 free_r= 0.2552
REMARK final r= 0.2282 free_r= 0.2589
REMARK rmsd bonds= 0.008400 rmsd angles= 1.10834
REMARK B rmsd for bonded mainchain atoms= 1.208 target= 1.5
REMARK B rmsd for bonded sidechain atoms= 1.951 target= 2.0
REMARK B rmsd for angle mainchain atoms= 2.032 target= 2.0
REMARK B rmsd for angle sidechain atoms= 3.019 target= 2.5
REMARK target= mlf final wa= 4.24416
REMARK final rweight= 0.0845 (with wa= 4.24416)
REMARK md-method= torsion annealing schedule= constant
REMARK starting temperature= 1600 total md steps= 1 * 100
REMARK cycles= 2 coordinate steps= 20 B-factor steps= 10
REMARK sg= P2(1)2(1)2(1) a= 55.680 b= 66.423 c= 68.253
REMARK alpha= 90 beta= 90 gamma= 90
REMARK topology file 1 : CNS_TOPPAR:protein.top
REMARK topology file 2 : CNS_TOPPAR:dna-rna.top
REMARK topology file 3 : CNS_TOPPAR:water.top
REMARK topology file 4 : CNS_TOPPAR:ion.top
REMARK topology file 5 : DHT.top
REMARK parameter file 1 : CNS_TOPPAR:protein_rep.param
REMARK parameter file 2 : CNS_TOPPAR:dna-rna_rep.param
REMARK parameter file 3 : CNS_TOPPAR:water_rep.param
REMARK parameter file 4 : CNS_TOPPAR:ion.param
REMARK parameter file 5 : DHT.par
REMARK reflection file= ARA70.cv
REMARK ncs= none
REMARK B-correction resolution: 6.0 − 2.3
REMARK initial B-factor correction applied to fobs :
REMARK B11= −25.406 B22= 14.816 B33= 10.590
REMARK B12= 0.000 B13= 0.000 B23= 0.000
REMARK B-factor correction applied to coordinate array B: 0.035
REMARK bulk solvent: density level= 0.320211 e/A\hat{ }3, B-factor= 27.8632 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 11733 ( 100.0 % ) |
| REMARK number of unobserved reflections (no entry or \|F\|=0): | 852 ( 7.3 % ) |
| REMARK number of reflections rejected: | 0 ( 0.0 % ) |
| REMARK total number of reflections used: | 10881 ( 92.7 % ) |
| REMARK number of reflections in working set: | 10321 ( 88.0 % ) |
| REMARK number of reflections in test set: | 560 ( 4.8 % ) |

CRYST1 55.680 66.423 68.253 90.00 90.00 90.00 P 21 21 21
REMARK VERSION: 1.1

Appendix 10Z

Header of PDB file containing atomic Coordinates for human AR LBD complexed with DHT and a GRIP-1 Box 3-derived peptide, designated CDP_{—}2, containing the LXXLL motif.

REMARK coordinates from minimization and B-factor refinement
REMARK refinement resolution: 30.0-2.07 A

| | |
|---|---|
| REMARK starting | r = 0.1963 free_r = 0.2316 |
| REMARK final | r = 0.1995 free_r = 0.2322 |

| | |
|---|---|
| REMARK rmsd bonds = 0.007219 rmsd angles = | 0.99998 |
| REMARK B rmsd for bonded mainchain atoms = | 1.527 target = 1.5 |
| REMARK B rmsd for bonded sidechain atoms = | 2.384 target = 2.0 |
| REMARK B rmsd for angle mainchain atoms = | 2.546 target = 2.0 |
| REMARK B rmsd for angle sidechain atoms = | 3.589 target = 2.5 |

REMARK target = mlf final wa = 1.65041
REMARK final rweight = 0.0734 (with wa = 1.65041)
REMARK md-method = torsion annealing schedule = constant
REMARK starting temperature = 2000 total md steps = 1 * 100
REMARK cycles = 2 coordinate steps = 20 B-factor steps = 10
REMARK sg = P2(1)2(1)2(1) a = 54.49 b = 67.37 c = 70.52
REMARK alpha = 90 beta = 90 gamma = 90
REMARK topology file 1 : CNS_TOPPAR:protein.top
REMARK topology file 2 : CNS_TOPPAR:dna-rna.top
REMARK topology file 3 : CNS_TOPPAR:water.top
REMARK topology file 4 : CNS_TOPPAR:ion.top
REMARK topology file 5 : DHT.top
REMARK parameter file 1 : CNS_TOPPAR:protein_rep.param
REMARK parameter file 2 : CNS_TOPPAR:dna-rna_rep.param
REMARK parameter file 3 : CNS_TOPPAR:water_rep.param
REMARK parameter file 4 : CNS_TOPPAR:ion.param
REMARK parameter file 5 : DHT.par
REMARK reflection file = Box3_C1.cv
REMARK ncs = none
REMARK B-correction resolution: 6.0-2.07
REMARK initial B-factor correction applied to fobs:
REMARK B11 = −7.614 B22 = 3.025 B33 = 4.589
REMARK B12 = 0.000 B13 = 0.000 B23 = 0.000
REMARK B-factor correction applied to coordinate array B: 0.440
REMARK bulk solvent: density level = 0.35949 e/A\hat{ }3, B-factor = 61.7478 A\hat{ }2
REMARK reflections with |Fobs|/sigma_F < 0.0 rejected
REMARK reflections with |Fobs| > 10000 * rms(Fobs) rejected

| | |
|---|---|
| REMARK theoretical total number of refl. in resol. range: | 16360 (100.0%) |
| REMARK number of unobserved reflections (no entry or \|F\| = 0): | 445 ( 2.7%) |
| REMARK number of reflections rejected: | 0 ( 0.0%) |
| REMARK total number of reflections used: | 15915 ( 97.3%) |
| REMARK number of reflections in working set: | 15136 ( 92.5%) |
| REMARK number of reflections in test set: | 779 ( 4.8%) |

CRYST1 54.490 67.370 70.520 90.00 90.00 90.00 P 21 21 21
REMARK VERSION: 1.1

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed:

1. A method of identifying a compound that modulates nuclear receptor activity, the method comprising:
modeling a test compound that fits spatially into an atomic structural model of the androgen receptor coactivator binding site or portion thereof, wherein said atomic structural model comprises atomic coordinates of an androgen receptor coactivator binding site and a molecule bound to the coactivator binding site; and
screening said test compound in an assay characterized by binding of the test compound to the coactivator binding site of a nuclear receptor, thereby identifying a test compound that modulates nuclear receptor activity.

2. The method of claim 1 wherein nuclear receptor activity is measured by binding of a coactivator to the coactivator binding site.

3. The method of claim 1 wherein nuclear receptor activity is measured by the suppression of transcriptional activity.

4. The method of claim 1 wherein nuclear receptor activity is measured by inhibition of coactivator binding.

5. The method of claim 1 wherein said screening is in vitro.

6. The method of claim 5 wherein said screening is high throughput screening.

7. The method of claim 1 wherein said atomic structural model of the human androgen receptor comprise coordinates of amino acid residues Leu 712, Val 713, Val716, Lys720, Phe725, Gln 733, Met734, Ile737, Gln738, Trp741, Glu 893, Met894, Glu 897, and Ile898.

8. The method of claim 1 wherein said test compound is a small organic molecule, a peptide, or a peptidomimetic.

9. The method of claim 1 wherein said test compound is an antagonist of coactivator binding.

10. The method of claim 1 wherein said nuclear receptor is selected from the group consisting of estrogen receptors, thyroid receptors, retinoid receptors, glucocorticoid receptors, progestin receptors, mineralocorticoid receptors, androgen receptors, peroxisome receptors and vitamin D receptors.

11. The method of claim 1 wherein the modeling comprises providing the atomic coordinates of the androgen receptor coactivator binding site to a computerized modeling system.

12. The method of claim 1 wherein said atomic structural coordinates are found in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith.

13. The method of claim 1 wherein said atomic structural coordinates further comprise a portion of the androgen receptor ligand binding domain.

14. The method of claim 12 wherein said atomic structural coordinates further comprise coordinates of a ligand bound to the ligand binding domain.

15. The method of claim 13 wherein said ligand is a hormone.

16. The method of claim 13 wherein said ligand is an agonist of androgen receptor activity.

17. The method of claim 1 wherein said molecule is a peptide.

18. The method of claim 17 wherein said peptide comprises a motif whose sequence is Z₁XXZ₂Z₃, wherein Z₁ and Z₃ are each independently F, L, W, or Y, and Z₂ is L, F, V, or Y, and X is any amino acid residue.

19. The method of claim 18 wherein the motif consists of residue sequences selected from the group consisting of: FXXLF, WXXLF, FXXFF, FXXLY, FXXYF, WXXVW, and FXXLW, wherein X is any amino acid.

20. The method of claim 17 wherein said modeling further comprises overlapping an atomic model of the test compound with the coordinates of the peptide.

21. The method of claim 18 wherein said modeling comprises identifying a fragment of said test molecule that fits into a cleft in said coactivator binding site that is occupied by the Z₁+1 residue of said peptide.

22. The method of claim 18 wherein said modeling comprises identifying a fragment of said test molecule that fits into a cleft in said coactivator binding site that is occupied by the Z₃+5 residue of said peptide.

23. The method of claim 18 wherein said test compound interacts with at least one residue selected from the group consisting of: Leu 712, Val 716, Met 734, Gln 738, Met 894, and Ile 898.

24. The method of claim 18 wherein said test compound interacts with at least one residue selected from the group consisting of: Val 716, Lys 720, Phe 725, Val 730, Gln 733, Ile 737.

25. The method of claim 1 wherein said test molecule is selected from a library of molecules.

26. The method of claim 1 wherein said test molecule is constructed from at least two fragments that are overlapped with the molecule bound to the coactivator binding site.

27. A method of identifying a compound that modulates nuclear receptor activity, the method comprising:
screening a test compound in an assay characterized by binding of a test compound to the coactivator binding site of a nuclear receptor, wherein the test compound has been modeled by spatially fitting an atomic model of the test compound into an atomic structural model of a portion of the androgen receptor coactivator binding site, wherein said atomic structural model comprises atomic coordinates of amino acid residues of the androgen receptor coactivator binding site and a molecule bound to the coactivator binding site, thereby identifying a test compound that modulates nuclear receptor activity.

28. The method of claim 27 wherein said nuclear receptor is selected from the group consisting of estrogen receptors, thyroid receptors, retinoid receptors, glucocorticoid receptors, progestin receptors, mineralocorticoid receptors, androgen receptors, peroxisome receptors and vitamin D receptors.

29. The method of claim 27 wherein said screening is in vitro.

30. The method of claim 27 wherein said screening is high throughput screening.

31. The method of claim 27 wherein said atomic coordinates of the human androgen receptor comprise coordinates of amino acid residues Leu 712, Val 713, Val716, Lys720, Phe725, Gln 733, Met734, Ile737, Gln738, Trp741, Glu 893, Met894, Glu 897, and Ile898.

32. The method of claim 27 wherein said test compound is an antagonist of coactivator binding.

33. The method of claim 27 wherein said test compound is a small organic molecule, a peptide, or a peptidomimetic.

34. The method of claim 27, wherein said atomic structural model is defined by the set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

35. A method of identifying an antagonist of coactivator binding to a nuclear receptor, the method comprising:
modeling a test compound which fits spatially into an atomic structural model of the androgen receptor coactivator binding site wherein the atomic structural model comprises atomic coordinates of amino acid residues of human androgen receptor coactivator binding site and a molecule bound to the coactivator binding site; and
screening said test compound in an assay for nuclear receptor activity, thereby identifying a compound which decreases the activity of the nuclear receptor by binding the coactivator binding site of said nuclear receptor.

36. The method of claim 35 wherein said nuclear receptor is selected from the group consisting of estrogen receptors, thyroid receptors, retinoid receptors, glucocorticoid receptors, progestin receptors, mineralocorticoid receptors, androgen receptors, peroxisome receptors, and vitamin D receptors.

37. The method of claim 35 wherein said atomic coordinates include the amino acid residues of human androgen receptor Leti 712, Val 713, Val716, Lys 720, Phe725, Gln 733, Met734, Ile737, Gln738, Trp741, Glu 893, Met894, Glu 897, and Ile898.

38. The method of claim 35 wherein said test compound contacts at least one residue selected from the group consisting of: Leu 712, Val 716, Met 734, Gln 738, Met 894, and Ile 898.

39. The method of claim 35 wherein said test compound contacts at least one residue selected from the group consisting of: Val 716, Lys 720, Phe 725, Val 730, Gln 733, Ile 737.

40. The method of claim 35 wherein the modeling comprises providing the atomic coordinates of an androgen receptor coactivator binding site and a molecule bound to the coactivator binding site to a computerized modeling system.

41. The method of claim 35 wherein the atomic structural model is experimentally derived.

42. The method of claim 35 wherein the atomic structural model has a resolution of better than 2.00 Å.

43. The method of claim 35, wherein said atomic structural model additionally comprises atomic coordinates of a ligand molecule bound to the ligand binding domain.

44. The method of claim 43, wherein said ligand is an androgen receptor agonist.

45. The method of claim 35 wherein the atomic structural model has coordinates presented in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

46. A method of identifying a compound that modulates androgen receptor activity, said method comprising:
modeling a test compound that fits spatially into an atomic structural model of an androgen receptor coactivator binding site, wherein said atomic structural model comprises atomic coordinates of amino acid residues of the androgen receptor coactivator binding site, and a molecule bound to the coactivator binding site; and
screening said test compound in an assay characterized by binding of the test compound to the androgen receptor coactivator binding site, thereby identifying a compound that modulates coactivator binding to the androgen receptor.

47. The method of claim 46 wherein the modeling comprises providing the atomic coordinates of an androgen receptor coactivator binding site and a molecule bound to the coactivator binding site to a computerized modeling system.

48. The method of claim 46, wherein said atomic structural model additionally comprises atomic coordinates of a ligand molecule bound to the ligand binding domain.

49. The method of claim 48, wherein said ligand is an androgen receptor agonist.

50. The method of claim 46 wherein the atomic structural model is experimentally derived.

51. The method of claim 46 wherein the atomic structural model has a resolution of better than 2.00 Å.

52. The method of claim 46 wherein the atomic structural model has coordinates presented in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

53. The method of claim 46 wherein said atomic coordinates of the human androgen receptor comprise coordinates of amino acid residues Leu 712, Val 713, Val716, Lys720, Phe725, Gln 733, Met734, Ile737, Gln738, Trp741, Glu 893, Met894, Glu 897, and Ile898.

54. The method of claim 46 wherein said test molecule contacts at least one residue selected from the group consisting of: Leu 712, Val 716, Met 734, Gln 738, Met 894, and Ile 898.

55. The method of claim 46 wherein said test molecule contacts at least one residue selected from the group consisting of: Val 716, Lys 720, Phe 725, Val 730, Gln 733, Ile 737.

56. A method of identifying an antagonist of coactivator binding to an androgen receptor, said method comprising:
modeling a test compound that fits spatially into the androgen receptor coactivator binding site using an atomic structural model of the androgen receptor coactivator binding site, wherein said atomic structural model comprises coordinates of the androgen receptor coactivator binding site, and coordinates of a coactivator bound to said coactivator binding site, and
screening said test compound in an assay characterized by binding of a test compound to the nuclear receptor coactivator binding site, thereby identifying a compound that inhibits coactivator binding to the androgen receptor.

57. The method of claim 56 wherein the modeling comprises providing the atomic coordinates of an androgen receptor coactivator binding site and a molecule bound to the coactivator binding site to a computerized modeling system.

58. The method of claim 56 wherein the atomic structural model has coordinates presented in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

59. The method of claim 56 wherein the atomic structural model is experimentally derived.

60. The method of claim 56 wherein the atomic structural model has a resolution of better than 2.00 Å.

61. The method of claim 56 wherein said atomic structural model additionally comprises coordinates of a ligand bound to said ligand binding domain.

62. The method of claim 61 wherein said ligand is an androgen receptor agonist.

63. The method of claim 56 wherein said atomic coordinates of the human androgen receptor comprise coordinates of amino acid residues Leu 712, Val 713, Val716, Lys720,Phe725, Gln 733, Met734, Ile737, Gln738, Trp741, Glu 893, Met894, Glu 897, and Ile898.

64. The method of claim 56 wherein said test molecule contacts at least one residue selected from the group consisting of: Leu 712, Val 716, Met 734, Gln 738, Met 894, and Ile 898.

65. The method of claim 56 wherein said test molecule contacts at least one residue selected from the group consisting of: Val 716, Lys 720, Phe 725, Val 730, Gln 733, Ile 737.

66. A computational method of designing an inhibitor of androgen receptor coactivator binding, comprising: fitting an atomic model of the compound into an atomic structural model of the coactivator binding site of the androgen receptor, wherein said compound consists of a first moiety that fits into a first cleft on the coactivator binding site and contacts at least one residue selected from the group consisting of Leu 712, Val 716, Met 734, Gln 738, Met 894, and Ile 898, and a second moiety that fits into a second cleft on the coactivator binding site, and contacts at least one residue selected from the group consisting of Val 716, Lys 720, Phe 725, Val 730, Gln 733, Ile 737, wherein said first moiety and said second moiety are joined by a linking group.

67. The method of claim 66 wherein said atomic structural model has coordinates in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

68. The method of claim 66 wherein the compound additionally makes a hydrogen bonding interaction with at least one residue selected from the group consisting of: Lys 720, Glu 897, and Gln 733.

69. The method of claim 66 wherein said contacts between said first moiety and said amino acid residue include a hydrogen bonding interaction, electrostatic interaction, van der Waals interaction, or a hydrophobic interaction.

70. The method of claim 66 wherein said contacts between said first moiety and said amino acid residue include a hydrogen bonding interaction, electrostatic interaction, van der Waals interaction or a hydrophobic interaction.

71. The method of claim 66 wherein said androgen receptor is selected from the group consisting of: human, chimpanzee, rat, and mouse.

72. A method of modulating androgen receptor activity in a mammal by administering to a mammal in need thereof a sufficient amount of a compound that fits spatially and preferentially into a coactivator binding site of the androgen receptor, wherein said compound is designed by a computational method that involves fitting an atomic model of the compound into an atomic structural model of the coactivator binding site of the androgen receptor, and wherein said compound consists of a first moiety that fits into a first cleft on the coactivator binding site and contacts at least one residue selected from the group consisting of Leu 712, Val 716, Met 734, Gln 738, Met 894, and Ile 898, and a second moiety that fits into a second cleft on the coactivator binding site, and contacts at least one residue selected from the group consisting of Val 716, Lys 720, Phe 725, Val 730, Gln 733, Ile 737, wherein said first moiety and said second moiety are joined by a linking group.

73. The method of claim 72 wherein the compound additionally makes a hydrogen bonding interaction with at least one residue selected from the group consisting of: Lys 720, Glu 897, and Gln 733.

74. The method of claim 72 wherein said compound inhibits an endogenous coregulator from binding to the coactivator binding site.

75. The method of claim 72 wherein the compound has been desired using an atomic structural model of the coactivator binding site of the androgen receptor ligand that has a set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

76. A method of inhibiting the binding of a coactivator to an androgen receptor, said method comprising:
contacting a molecule with a coactivator binding site on the androgen receptor, wherein the molecule fits spatially into the coactivator binding site, and wherein the molecule binds more strongly to the receptor than does the coactivator.

77. The method of claim 76, wherein the molecule consists of a first moiety that fits into a first cleft on the coactivator binding site and contacts at least one residue selected from the group consisting of Leu 712, Val 716, Met 734, Gln 738, Met 894, and Ile 898, and a second moiety that fits into a second cleft on the coactivator binding site, and contacts at least one residue selected from the group consisting of Val 716, Lys 720, Phe 725, Val 730, Gln 733, Ile 737, and Met 734, wherein said first moiety and said second moiety are joined by a linking group.

78. The method of claim 77 wherein the molecule additionally makes a hydrogen bonding interaction with at least one residue selected from the group consisting of: Lys 720, Glu 897, and Gln 733.

79. The method of claim 76, wherein the molecule is a peptide that comprises a motif whose sequence is Z₁XXZ₂Z₃, wherein Z₁ and Z₃ are each independently F, L, W, or Y, and Z₂ is L, F, V, or Y, and X is any amino acid residue.

80. The method of claim 76, wherein the motif consists of residue sequences selected from the group consisting of: FXXLF, WXXLF, FXXFF, FXXLY, FXXYF, WXXVW, and FXXLW, wherein X is any amino acid.

81. The method of claim 76 wherein the molecule has been designed using an atomic structural model of the coactivator binding site of the androgen receptor ligand that has a set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

82. A method of modulating the activity of an androgen receptor, said method comprising:
contacting a molecule with a coactivator binding site on the androgen receptor, wherein the molecule fits spatially into the coactivator binding site, and wherein the molecule has been designed by modeling at least one test compound into an atomic structural model of the coactivator binding site of the androgen receptor, wherein said atomic structural model comprises atomic coordinates of amino acid residues of the androgen receptor coactivator binding site and a second molecule bound to the coactivator binding site.

83. The method of claim 82 wherein said compound inhibits an endogenous coregulator from binding to the coactivator binding site.

84. The method of claim 82 wherein the atomic structural model of the coactivator binding site of the androgen receptor ligand has a set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

85. A method of modulating androgen receptor activity in a mammal by administering to a mammal in need thereof a sufficient amount of a compound that fits spatially and preferentially into a coactivator binding site of the androgen receptor, wherein said compound is designed by fitting an atomic model of the compound into an atomic structural model of the coactivator binding site of the androgen receptor, wherein said atomic structural model comprises atomic coordinates of amino acid residues of the androgen receptor coactivator binding site and a second molecule bound to the coactivator binding site.

86. The method of claim 85 wherein said compound inhibits an endogenous coregulator from binding to the coactivator binding site.

87. The method of claim 85 wherein the atomic structural model of the coactivator binding site of the androgen receptor ligand nas a set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

88. A machine-readable data storage medium encoded with machine readable data which, when using a machine programmed with instructions for using said data, is capable of causing a graphical three-dimensional representation of a molecular complex to be displayed, comprising: at least a portion of an androgen receptor ligand binding domain, including an androgen receptor coactivator binding site; a molecule bound to the androgen receptor coactivator binding site; and a ligand bound to the ligand binding domain.

89. The machine-readable data storage medium of claim 88 wherein the androgen receptor ligand binding domain is a homologue having a root mean square deviation of not more than 1.5 Å from the backbone atoms of said amino acids in the ligand binding domain of AR in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith.

90. The machine-readable data storage medium of claim 88 wherein said machine readable data comprises a set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith.

91. The machine-readable data storage medium of claim 88, wherein said androgen receptor is human.

92. The machine-readable data storage medium of claim 88, wherein said molecule is a peptide.

93. The machine-readable data storage medium of claim 88, wherein said peptide comprises a Nuclear Receptor Box amino acid sequence or derivative thereof.

94. Use of a machine-readable data storage medium, comprising machine readable data, in conjunction with a machine programmed with instructions for using said data, for identifying a molecule that modulates coactivator binding to an androgen receptor, wherein the computer displays a graphical three-dimensional representation of a complex of the molecule bound to a coactivator binding site of the androgen receptor, and wherein the data comprises structure coordinates in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith.

95. The use of claim 94, wherein the androgen receptor ligand binding domain is a homologue having a root mean square deviation of not more than 1.5 Å from the backbone atoms of the amino acids of the androgen receptor ligand binding domain defined by a set of structure coordinates depicted in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith.

96. A cocrystal comprising:
a portion of an androgen receptor ligand binding domain;
a ligand bound to the ligand binding domain of the receptor; and
a coactivator bound to a coactivator binding site of the receptor.

97. The cocrystal of claim 96 wherein said cocrystal diffracts with at least 1.9 Å resolution.

98. The cocrystal of claim 96 wherein said androgen receptor is human.

99. The cocrystal of claim 96 wherein said androgen receptor is a homolog of the human androgen receptor.

100. The cocrystal of claim 96 wherein said ligand is a naturally occurring hormone.

101. The cocrystal of claim 96 wherein said coactivator is a peptide.

102. The cocrystal of claim 101 wherein said peptide comprises a NR-box amino acid sequence.

103. The cocrystal of claim 101 wherein said peptide comprises a motif whose sequence is Z₁XXZ₂Z₃, wherein Z₁ and Z₃ are each independently F, L, W, or Y, and Z₂ is L, F, V, or Y, and X is any amino acid residue.

104. The cocrystal of claim 103 wherein said peptide is a coactivator-derived peptide.

105. The cocrystal of claim 103 wherein said peptide consists of 15 amino acid residues.

106. The cocrystal of claim 103 wherein said motif consists of residue sequences selected from the group consisting of: FXXLF, WXXLF, FXXFF, FXXLY, WXXVW, FXXYF, and FXXLW.

107. The cocrystal of claim 96 having the structure defined by the structural coordinates as shown in any one of Table 1 (A) and (B), and Table 2 (A)-(H), found respectively in the files identified as Table1_ARLBD_DHT_CDP.txt and Table2_ARLBD_DHT_CRP.txt, presented on CD-R herewith, or a homologue thereof, said homologue having a root mean square deviation from the backbone atoms of said amino acids of not more than 1.5 Å.

108. A cocrystal consisting of:
an androgen receptor ligand binding domain;
a ligand bound to the ligand binding domain of the receptor;
a coactivator bound to a coactivator binding site of the receptor; and
crystallographically bound water.

109. An isolated and purified protein complex comprising:
a portion of an androgen receptor ligand binding domain;
a ligand bound to the ligand binding domain of the receptor; and
a coactivator bound to a coactivator binding site of the receptor.

110. An isolated and purified homolog of the protein complex of claim 109.

111. The isolated and purified protein complex of claim 109, wherein said coactivator is a peptide that comprises a motif whose sequence is Z₁XXZ₂Z₃, wherein Z₁ and Z₃ are each independently F, L, W, or Y, and Z₂ is L, F, V, or Y, and X is any amino acid residue.

112. An isolated and purified protein complex consisting of:
an androgen receptor ligand binding domain;
a ligand bound to the ligand binding domain of the receptor;
a coactivator bound to a coactivator binding site of the receptor; and
at least one molecule of solvent bound thereto.

113. An isolated and purified polypeptide consisting of a portion of the human androgen receptor starting at amino acid residue 669 and ending at amino acid residue 918, as set forth in SEQ ID NO: 29, bound to a ligand, and bound to a coactivator.

114. An isolated and purified homolog of the polypeptide of claim 113.

115. The isolated and purified polypeptide of claim 113, wherein said coactivator is a peptide that comprises a motif whose sequence is Z₁XXZ₂Z₃, wherein Z₁ and Z₃ are each independently F, L, W, or Y, and Z₂ is L, F, V, or Y, and X is any amino acid residue.

116. A compound of formula:

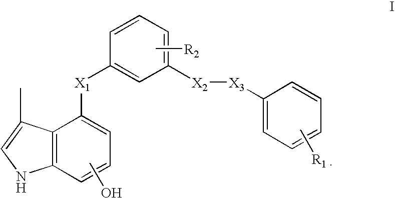

117. A method of treating prostate cancer by administering a pharmaceutical composition comprising a compound of formula: [chemical structure]
